(19) United States
(12) Patent Application Publication
(10) Pub. No.: US 2016/0264985 A1
(43) Pub. Date: Sep. 15, 2016

(54) MICROORGANISMS FOR FATTY ACID PRODUCTION USING ELONGASE AND DESATURASE ENZYMES

(71) Applicant: Synthetic Genomics, Inc., La Jolla, CA (US)

(72) Inventors: Nicky C. Caiazza, Rancho Santa Fe, CA (US); Elizabeth A. Felnagle, Seattle, WA (US); Jun Urano, Irvine, CA (US); Maung N. Win, San Diego, CA (US); Randor R. Radakovits, Escondido, CA (US)

(21) Appl. No.: 15/068,448

(22) Filed: Mar. 11, 2016

Related U.S. Application Data
(60) Provisional application No. 62/132,409, filed on Mar. 12, 2015.

(51) Int. Cl. C12N 15/82; C12N 9/00; C12N 9/02

(52) U.S. Cl. CPC C12N 15/8247; C12N 9/0071; C12N 9/93; A23K 1/007; A23K 1/164; C12Y 114/19; C12Y 602/01003

(57) ABSTRACT

Recombinant microorganisms engineered for the production of polyunsaturated fatty acids (PUFAs) are provided. Also provided are biomass, microbial oils, and food products and ingredients produced by or comprising the microorganisms of the invention.

### CROSS-REFERENCE TO RELATED APPLICATIONS

This application claims the benefit of provisional application Ser. No. 62/132,409, filed Mar. 12, 2015, the entire contents of which is incorporated herein by reference in its entirety.

### INCORPORATION OF SEQUENCE LISTING

The material in the accompanying sequence listing is hereby incorporated by reference in this application. The accompanying sequence listing text file, name SGI1870_1_Sequence_Listing.txt., was created on Mar. 10, 2016, and is 146 kb. The file can be assessed using Microsoft Word on a computer that uses Windows OS.

### BACKGROUND

Omega-3 polyunsaturated fatty acids (PUFAs) are an essential component of the human and animal diet and are necessary for human and animal well-being. Some PUFAs, such as linoleic acid and alpha-linoleic acid cannot be synthesized by the human body and must be obtained through the diet. Fats not only enhance the taste and enjoyment of food, but some PUFAs can also be used to replace less healthy saturated fatty acids in the human diet, which may lower the risk of health problems such as coronary artery disease.

Commercial suppliers of omega-3 polyunsaturated fatty acids (PUFAs) have been in need of new sources for a sustainable supply of vegetarian, low mercury and high purity PUFAs. This is due to diminishing fish supplies as well to as expensive separations methods that are required to obtain PUFAs of sufficient purity. In response to this demand, algal and fungal fermentations have been developed using organisms that are naturally rich in either DHA or ARA, two common ingredients found in infant formula.

However, in the case of EPA, cost-effective algal or fungal fermentations are not available and can currently be economically obtained only from diminishing marine stocks. Marine fish and krill oils and their concentrates are a majority source of EPA and DHA for manufacturers and formulators of the dietary supplement, food and beverage, animal and pet feed, pharmaceutical, and clinical nutrition markets. The supplies for these markets are therefore subject to the variability of PUFA levels that occurs in marine sources. Current fish harvests are low in EPA, which therefore impacts products useful for improving cardiovascular health and reducing inflammation, as clinical studies have revealed a role for EPA in treating and preventing heart disease, as well as having anti-inflammatory properties. The low levels of EPA at the time of harvest will lead to products with poor EPA specifications that require expensive improvements to separate and concentrate EPA from DHA.

There is therefore a need for new and more cost-effective sources of PUFAs, including EPA. There is further a need for sustainable sources of PUFAs that are vegetarian, low in mercury, and of high purity.

### SUMMARY OF THE INVENTION

The present invention provides recombinant microorganisms engineered for the production of polyunsaturated fatty acids (PUFAs). The microorganisms can comprise one or more heterologous enzymes, for example at least one heterologous elongase and/or at least one heterologous desaturase. In some embodiments the product of at least one heterologous enzyme is the substrate of another heterologous enzyme and therefore an exogenous pathway is engineered into the microorganism for producing one or more PUFAs. In some embodiments the microorganism is a Labyrinthulomycetes cell, and the microorganism can contain one or more nucleic acids of the invention. In various embodiments the cells produce a FAME profile that is advantageous, for example by producing a high amount of EPA or other desirable PUFAs and a low amount of DHA. Also provided are biomass, microbial oils, and food products and ingredients produced by or comprising the microorganisms of the invention. The invention also provides methods for the production of all of the above.

In a first aspect the present invention provides a recombinant Labyrinthulomycetes cell for the production of one or more polyunsaturated fatty acids. The recombinant cells have at least one heterologous elongase and at least one heterologous desaturase functionally expressed in the recombinant cell. The enzymes perform at least one substrate to product elongase conversion step and at least one substrate to product desaturase conversion step, which steps can be selected from the steps disclosed herein.

In one embodiment the product of at least one heterologous enzyme is the substrate of at least one other heterologous enzyme. The recombinant cell can have at least three heterologous enzymes that perform at least three of the substrate to product conversion steps, and at least two of the products of the heterologous enzymes are the substrates for at least two of the heterologous enzymes. In some embodiments the recombinant cell is a Labyrinthulomycete from a genus of: an Aurantiochytrium, a Schizochytrium, a Thraustochytrium, and an Oblongichytrium. In one embodiment the at least three heterologous enzymes are expressed on one or more vectors.

In various embodiments the series of the substrate to product conversion steps converts LA to ARA. In one embodiment the enzymes perform the substrate to product conversion steps 18:2 (Δ9,12) (LA) into 18:3 (Δ6,9,12) (GLA) using a Δ6-desaturase; 18:3 (Δ6,9,12) (GLA) into 20:3 (Δ8,11,14) (DGLA) using a Δ6-elongase; and 20:3 (Δ8,11,14) (DGLA) into 20:4 (Δ5,8,11,14) (ARA) using a Δ5-desaturase; and thereby converts LA to ARA. The series can further perform a substrate to product conversion step of ARA into EPA.

In one embodiment the recombinant cell of the invention has enzymes that perform the substrate to product conversion steps: 18:3 (Δ6,9,12) (GLA) into 18:4(Δ6,9,12,15) (SDA) using an ω3-desaturase; 18:4 (Δ6,9,12,15) (SDA) into 20:4 (Δ8,11,14,17) (ETA) using a Δ6-elongase; 20:4 (Δ8,11,14,17) (ETA) into 20:5 (Δ5,8,11,14,17) (EPA) using a Δ5-desaturase; and thereby converts GLA to EPA.

In various embodiments the recombinant cell of the invention has heterologous enzymes that perform substrate to product conversion steps selected from a) or b) or c) or d) as follows: 18:2 (Δ9,12) (LA) into 18:3 (Δ6,9,12) (GLA) using a M-desaturase; and 18:3 (Δ6,9,12) (GLA) into 20:3 (Δ8,11,14) (DGLA) using a Δ6-elongase; and 20:3 (Δ8,11,14) (DGLA) into 20:4 (Δ5,8,11,14) (ARA) using a Δ5-desaturase; and 20:4 (Δ5,8,11,14) (ARA) into a 20:5(Δ5,8,11,14,17) (EPA) using an ω3-desaturase; or 18:2 (Δ9,12) (LA) into 18:3(Δ9,12,15) (ALA) using a ω3-desaturase; and 18:3 (Δ9,12,15) (ALA) into 18:4 (Δ6,9,12,15) (SDA) using a Δ6-desaturase; and 18:4 (Δ6,9,12,15) (SDA) into 20:4 (Δ8,11,14,17) (ETA) using a M-elongase; and 20:4 (Δ8,11,14,17) (ETA) into a 20:5(Δ5,8,11,14,17) (EPA) using an ω3-desaturase; or 18:2 (Δ9,12) (LA) into 18:3 (Δ6,9,12) (GLA) using a Δ6-desaturase; and 18:3 (Δ6,9,12) (GLA) into 18:4(Δ6,9,12,15) (SDA) using an ω3-desaturase; and 18:4 (Δ6,9,12,15) (SDA) into 20:4 (Δ8,11,14,17) (ETA) using a Δ6-elongase; and 20:4 (Δ8,11,14,17) (ETA) into 20:5 (Δ5,8,11,14,17) (EPA) using a Δ5-desaturase; or 18:2 (Δ9,12) (LA) into 18:3 (Δ6,9,12) (GLA) using a Δ6-desaturase; and 18:3 (Δ6,9,12) (GLA) into 20:3 (Δ8,11,14) (DGLA) using a Δ6-elongase; and 20:3 (Δ8,11,14) (DGLA) into 20:4 (Δ8,11,14,17) (ETA) using a Δ5-desaturase; and 20:4 (Δ8,11,14,17) (ETA) into a 20:5(Δ5,8,11,14,17) (EPA) using an ω3-desaturase; and thereby convert LA to EPA.

In additional embodiments any of the recombinant cells of the invention can further comprising the conversion steps 20:5 (Δ5,8,11,14,17) (EPA) into 22:5 (Δ7,10,13,16,19) (DPA) using a Δ5-elongase; and/or 22:5 (Δ7,10,13,16,19) (DPA) into 22:6 (Δ4,7,10,13,16,19) (DHA) using a Δ4-desaturase. The recombinant cells can also further perform the conversion steps 20:4 (Δ5,8,11,14) (ARA) into a 22:4 (Δ7,10,13,16) (DTA) using a Δ5-elongase; and/or 22:4 (Δ7,10,13,16) (DTA) into a 22:5 (Δ4,7,10,13,16) (DPAn6) using a Δ4-desaturase.

In one embodiment a recombinant cell of the invention produces a FAME profile having less than 25% DHA or less than 5% DHA, or less than 1% DHA. In various embodiments the recombinant cells of the invention can also produce OA, PA, ARA or EPA and produce a FAME profile having less than 10% DHA or less than 5% DHA or less than 1% DHA or no detectable DHA.

In some particular embodiments the recombinant cell produces a FAME profile having greater than 12% OA or greater than 12% ARA, or greater than 8% EPA. In one embodiment the recombinant cells or organisms of the invention do not require the presence of fatty acids in the medium to grow and remain viable. In one embodiment the recombinant cells do not require the presence of DHA in the medium to grow and remain viable.

In another aspect the invention provides a biomass comprised of a recombinant Labyrinthulomycetes cell as described herein. The biomass can have a FAME profile comprising a parameter selected from: greater than 8% EPA, greater than 12% ARA, greater than 12% OA, greater than 15% PA, and the parameter can be produced by an exogenous pathway. The biomass can also have a FAME profile of greater than 10% EPA. In some embodiments the biomass has a FAME profile of greater than 12% ARA, and can also have less than 10% DHA.

In another aspect the invention provides a food product or ingredient that comprises the biomass described herein.

In another aspect the invention provides a nucleic acid sequence having at least 90% sequence identity with a sequence of SEQ ID NO: 27-52 and having at least one substitution modification relative to the sequence found in SEQ ID NO: 27-52.

In another aspect the invention provides a nucleic acid vector for genetically transforming a cell. The vector contains a nucleic acid sequence having at least 90% sequence identity with a sequence of SEQ ID NO: 27-52 and having at least one substitution modification relative to the sequence found in SEQ ID NO: 27-52. The vector can have a promoter active in a Labyrinthulomycetes cell described herein. In one embodiment the promoter is Tubα-997. The vector can also have PGK1t as a terminator.

In another aspect the invention provides a recombinant Labyrinthulomycetes cell having at least one heterologous elongase and at least one heterologous desaturase that is functionally expressed in the cell. The heterologous enzymes can perform at least one substrate to product elongase conversion step and at least one substrate to product desaturase conversion step, and the heterologous elongase and/or desaturase have at least 90% sequence identity with a sequence of SEQ ID NO: 1-26 and having at least one substitution modification relative to the sequence found in SEQ ID NO: 1-26. At least one elongase and at least one desaturase are functionally expressed by an exogenous vector.

In another aspect the invention provides a recombinant Labyrinthulomycetes cell producing a FAME profile having greater than 12% ARA; or greater than 8% EPA; or greater than 20% SA; or greater than 10% OA; and less than 10% DHA or less than 5% DHA. The recombinant cell can also grow and be viable on a medium that is not supplemented with a PUFA.

In another aspect the invention provides a microbial oil containing at least one polyunsaturated fatty acid synthesized by a Labyrinthulomycetes cell. The oil can have a FAME profile having a content of EPA that is higher than the content of DHA. The oil can be produced by a Labyrinthulomycete as described herein. The oil can have a FAME profile with greater than 10% EPA and, optionally, less than 5% DHA. It can also have a FAME profile having greater than 10% EPA and less than 1% DHA. The microbial oil can be an extracted and unconcentrated oil. In another embodiment the microbial oil contains at least one polyunsaturated fatty acid synthesized by a Labyrinthulomycetes cell and has a FAME profile having a content of ARA of greater than 15% that, optionally, also has a DHA content of less than 5%.

In another aspect the invention provides a food product or food ingredient containing a microbial oil as described herein. In one embodiment the food product is animal feed.

In another aspect the invention provides a method of producing a high value oil or a biomass by cultivating a recombinant Labyrinthulomycetes cell having a FAME profile comprising a parameter selected from: greater than 12% ARA; greater than 8% EPA; greater than 20% SA; and greater than 10% OA, and wherein the parameter is produced by an exogenous pathway. The FAME profile can also have less than 5% DHA. In the method the cell can also be cultured on a medium that is not supplemented with a PUFA (e.g. DHA). In the method the recombinant cell can produce a FAME profile having greater than 12% ARA and/or greater than 8% EPA. The biomass is made from the cells produced by the method. The invention also provides a method of producing a food product or ingredient by including or incorporating into the food product or food ingredient a microbial oil or biomass of the invention.

In another aspect the invention provides a Labyrinthulomycetes cell that produces EPA from an exogenous recombinant pathway. The recombinant cell can have a native polyketide synthesis pathway that has been disrupted, deleted, or impaired, and the cell can produce a greater amount of EPA than DHA. The exogenous pathway can be an elongase/desaturase pathway or an exogenous polyketide synthesis pathway comprising bacterial enzymes. In one embodiment the cell grows on a media that does not contain a PUFA as a supplement. The cell can produce a FAME profile having less than 1% DHA and/or a FAME profile having greater than 8% EPA.

### DETAILED DESCRIPTION OF THE DRAWINGS

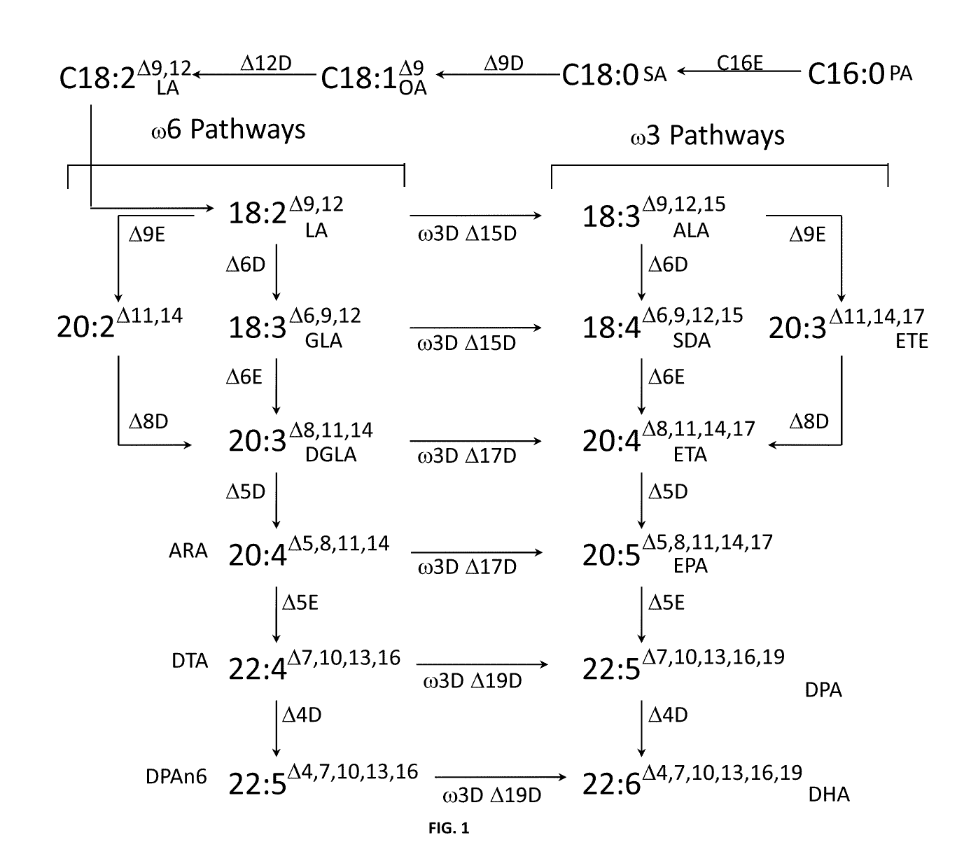

FIG. 1 is a schematic illustration of long chain polyunsaturated fatty acid biosynthesis using elongase and desaturase enzymes.

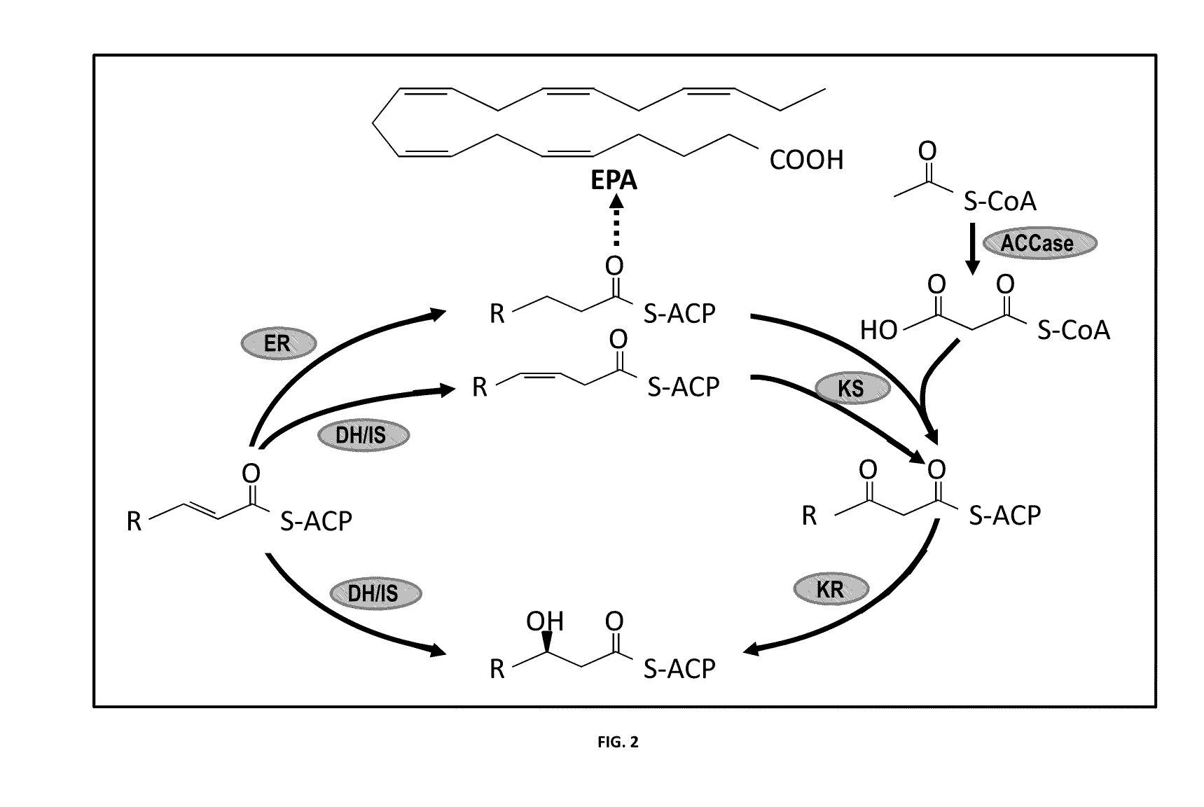

FIG. 2 is a schematic illustration of the polyketide (PKS) pathway for the formation of EPA.

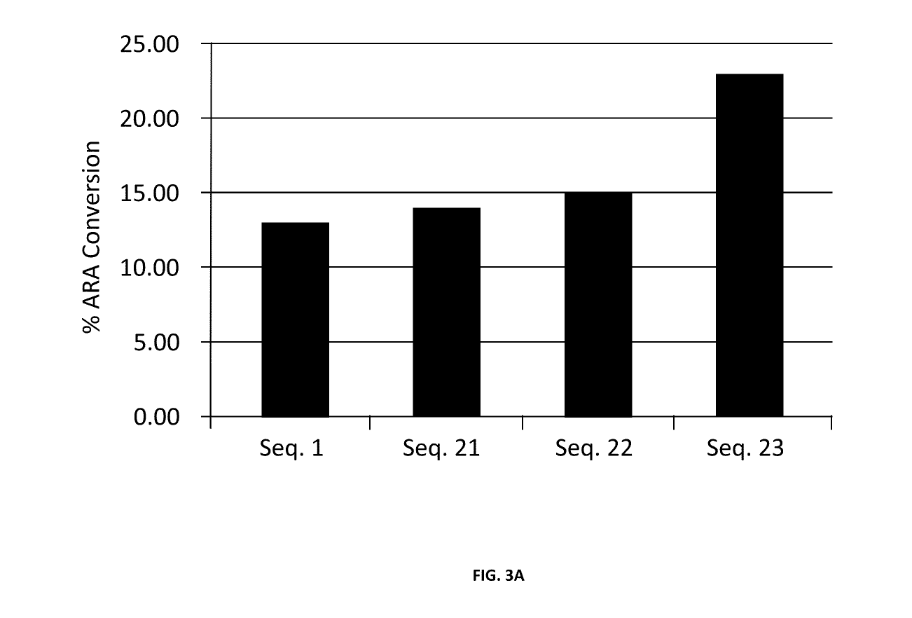

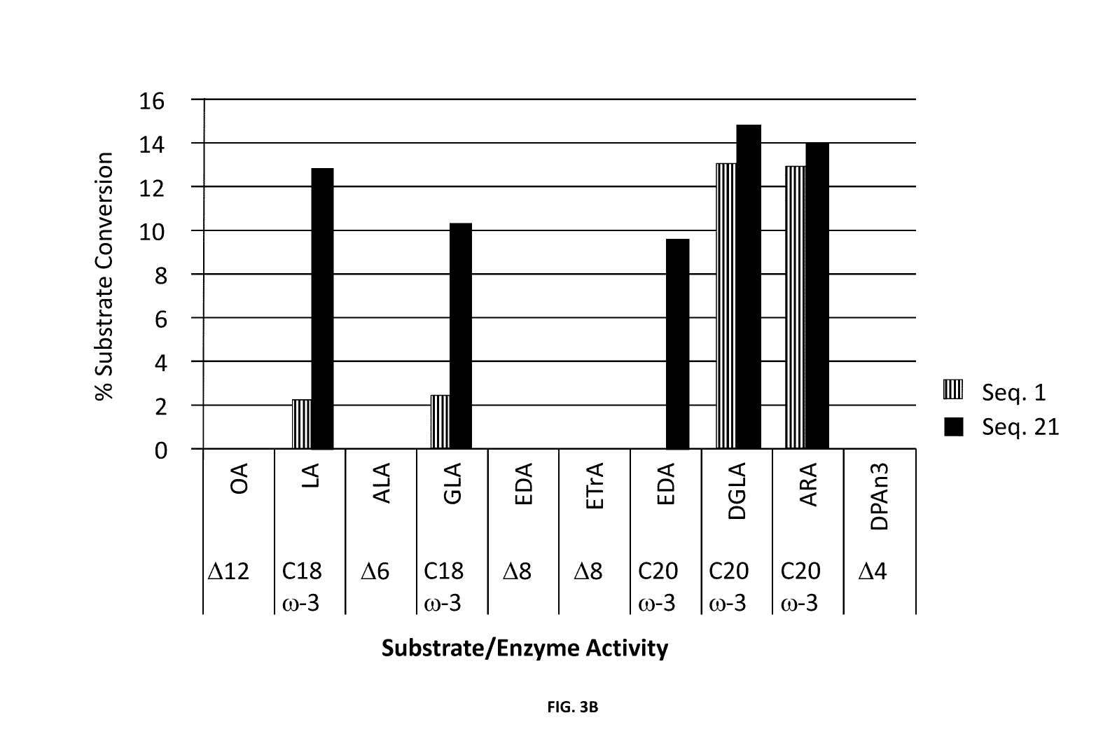

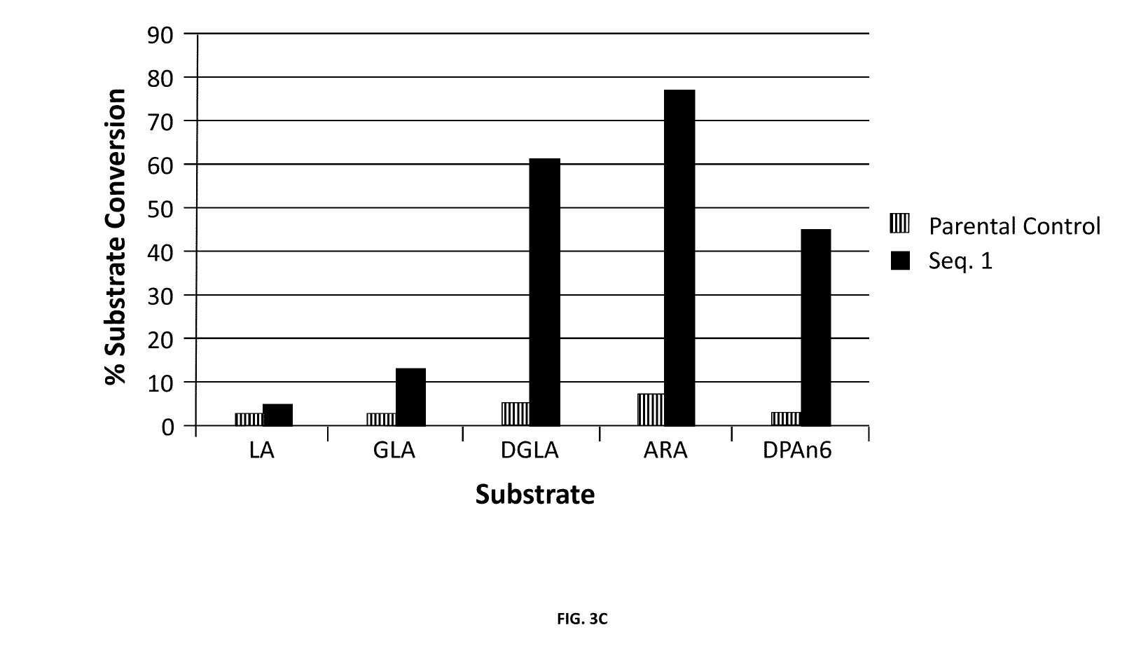

FIGS. 3A-3C provide charts showing (3A) the activity of omega-3 desaturases encoded by SEQ ID NOs: 1 and 21-23 in S. cerevisiae, (3B) specificities of SEQ ID NOs: 1 and 21 in S. cerevisiae (on the x-axis, the top wording indicates the substrate tested, the bottom wording indicates the corresponding enzyme activity), and (3C) activity of SEQ ID NO: 1 in an Aurantiochytrium PUFA auxotroph strain.

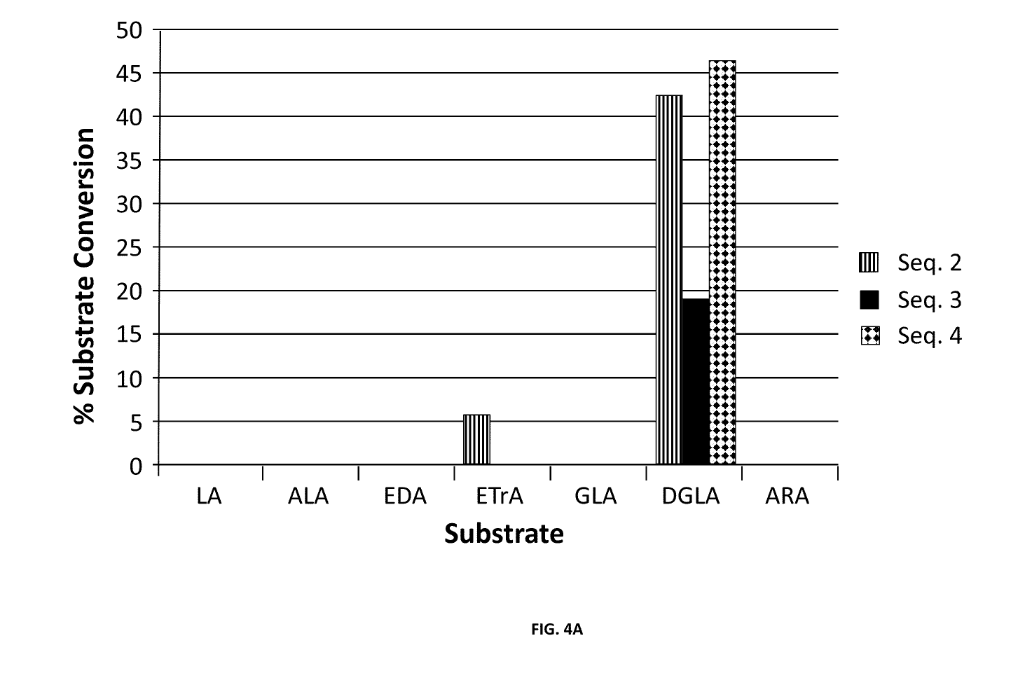

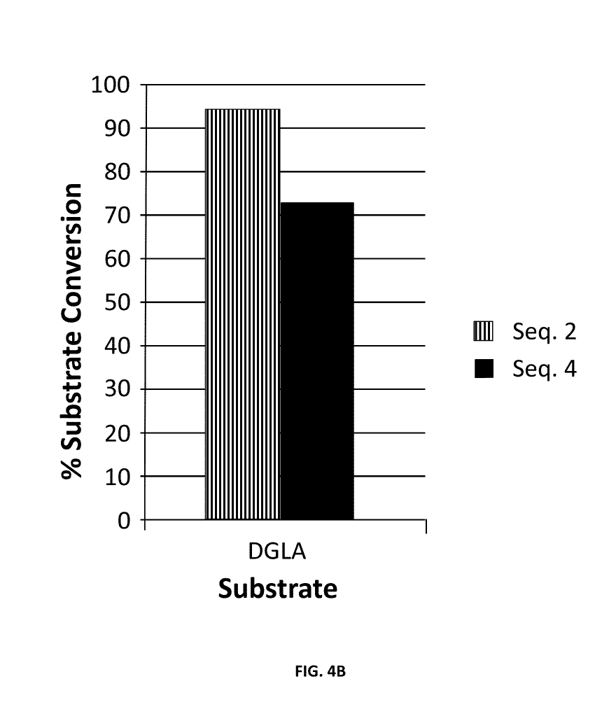

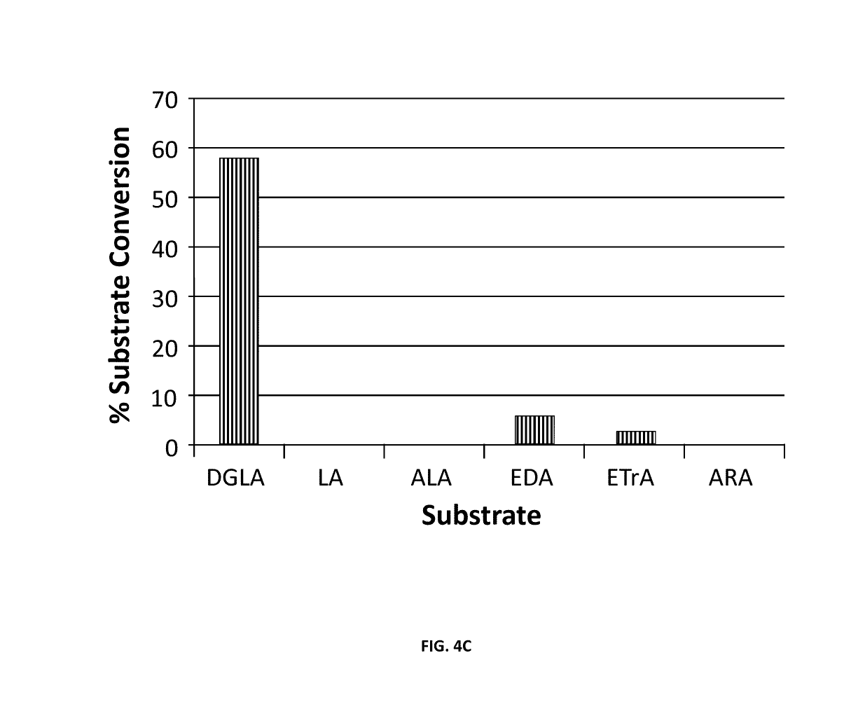

FIGS. 4A-4C provide bar charts showing (4A) the activity and specificity of the Δ5 desaturases encoded by SEQ ID NOs: 2-4 in S. cerevisiae, (4B) activity of the Δ5 desaturases encoded by SEQ ID NOs: 2 and 4 in an Aurantiochytrium PUFA auxotrophic strain, and (4C) specificity of the Δ5 desaturase encoded by SEQ ID NO: 2 in an Aurantiochytrium PUFA auxotrophic strain.

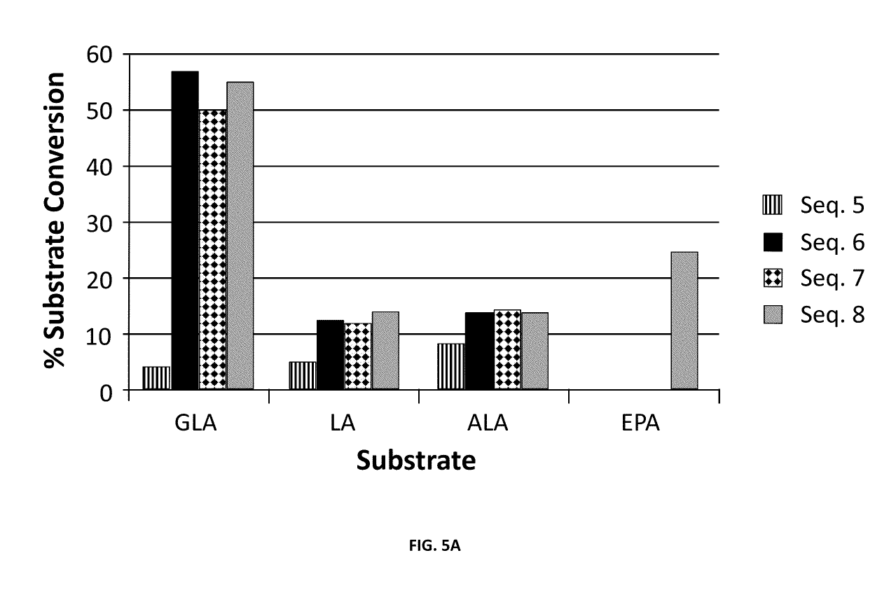

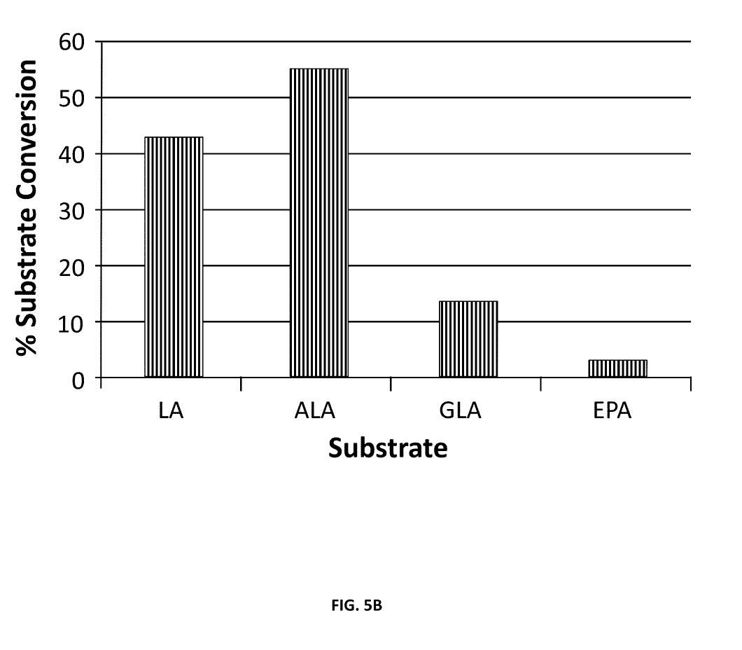

FIGS. 5A-5B provide bar charts showing (5A) the activity and specificity of the Δ6 elongases encoded by SEQ ID NOs: 5-8 in S. cerevisiae, and (5B) activity and specificity of the Δ6 elongase encoded by SEQ ID NO: 5 in an Aurantiochytrium PUFA auxotrophic strain.

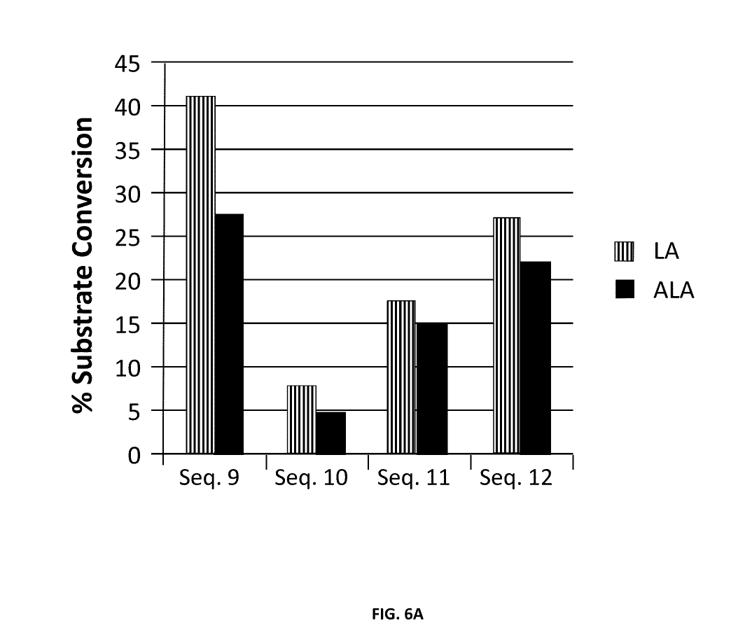

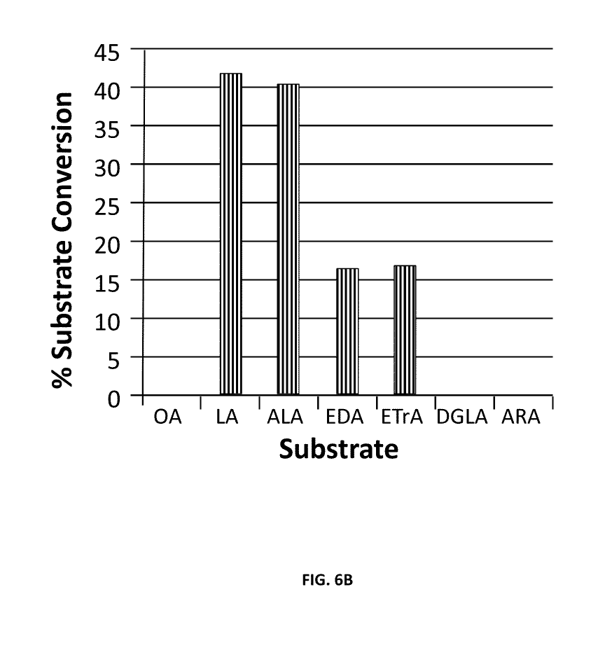

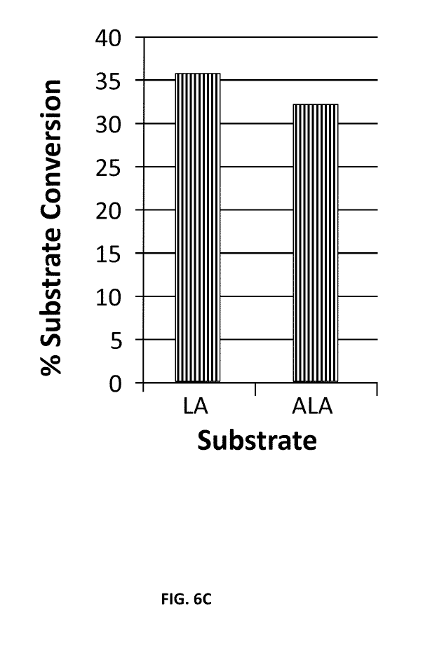

FIGS. 6A-6C provide bar charts showing (6A) the activity and specificity of the Δ6 desaturases encoded by SEQ ID NOs: 9-12 in S. cerevisiae, and (6B) activity and specificity of the Δ6 desaturase encoded by SEQ ID NO: 9 in S. cerevisiae and (6C) in an Aurantiochytrium PUFA auxotroph strain.

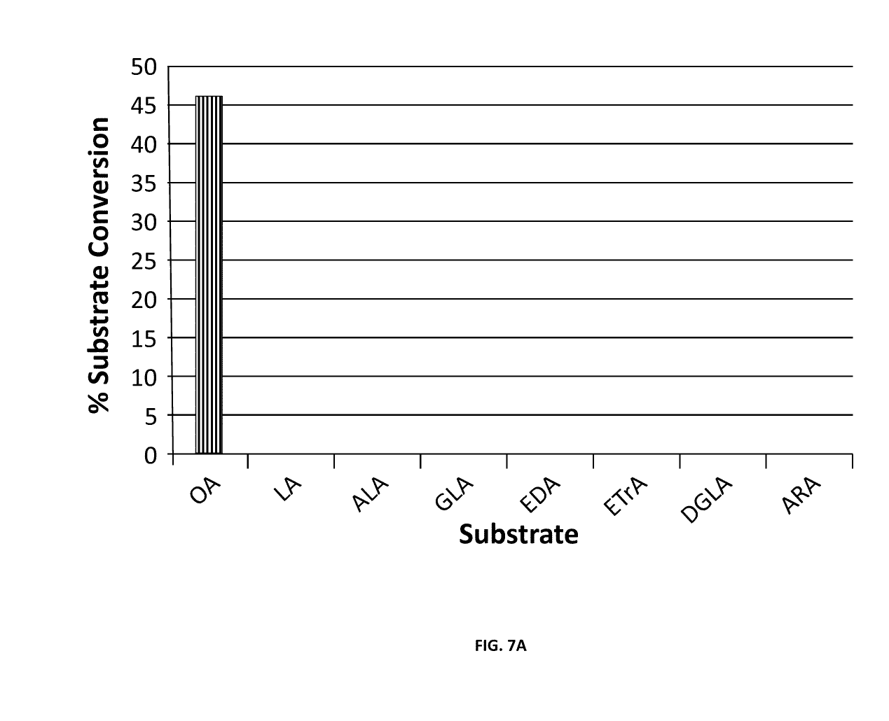

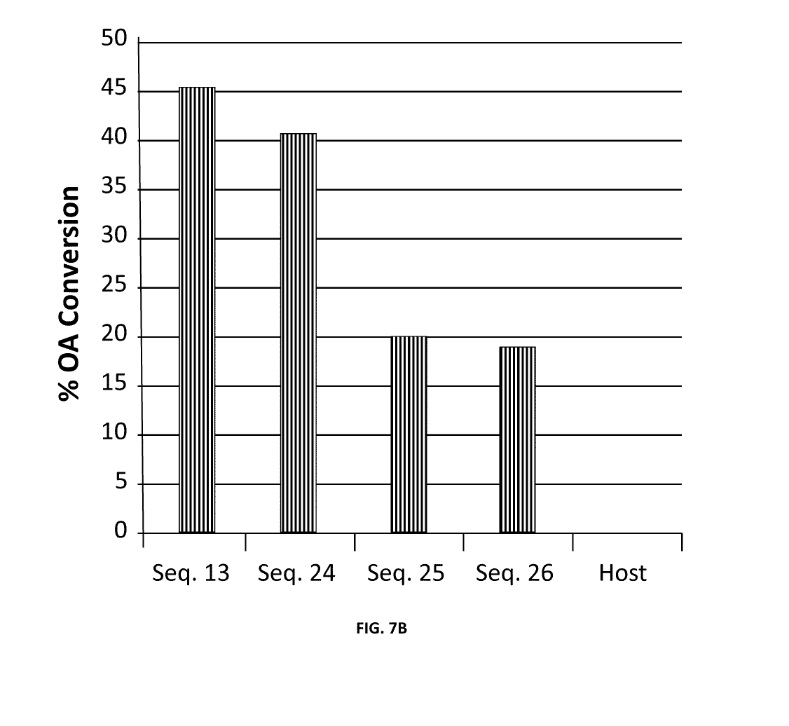

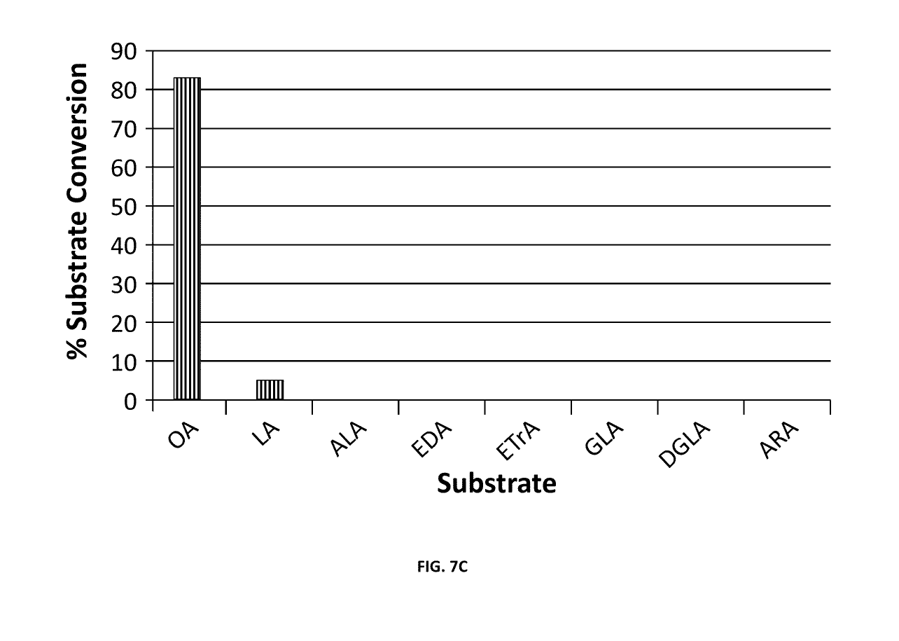

FIGS. 7A-7C provide bar charts showing (7A) the activity and specificity of the Δ12 desaturase encoded by SEQ ID NO: 13 in S. cerevisiae, (7B) additional Δ12 desaturases acting on endogenously produced OA in S. cerevisiae, and SEQ ID NO: 13 expressed in an Aurantiochytrium PUFA auxotrophic strain (7C).

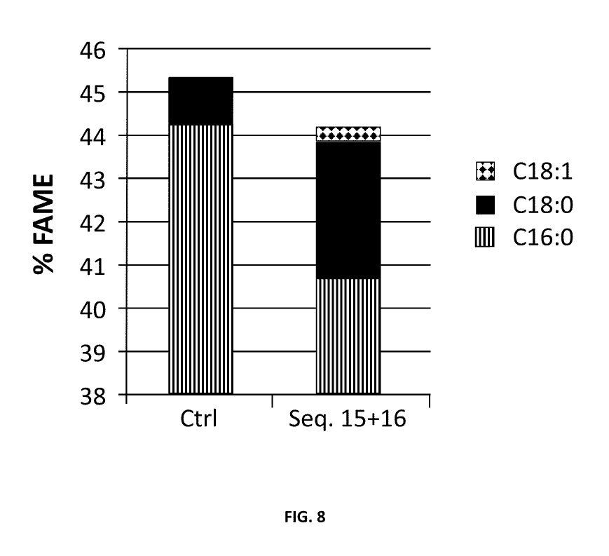

FIG. 8 provides a bar chart showing the activity of a co-expressed C16 elongase (SEQ ID NO: 16) and Δ9 desaturase (Seq. 15) in Aurantiochytrium.

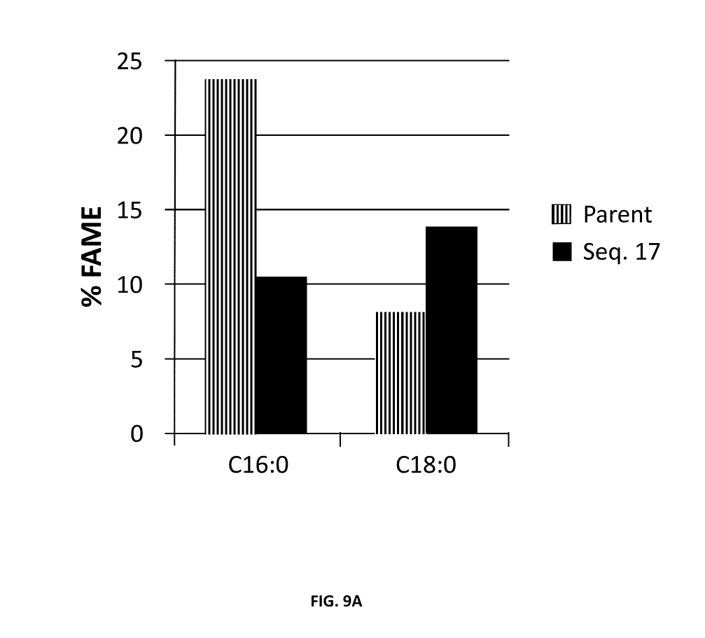

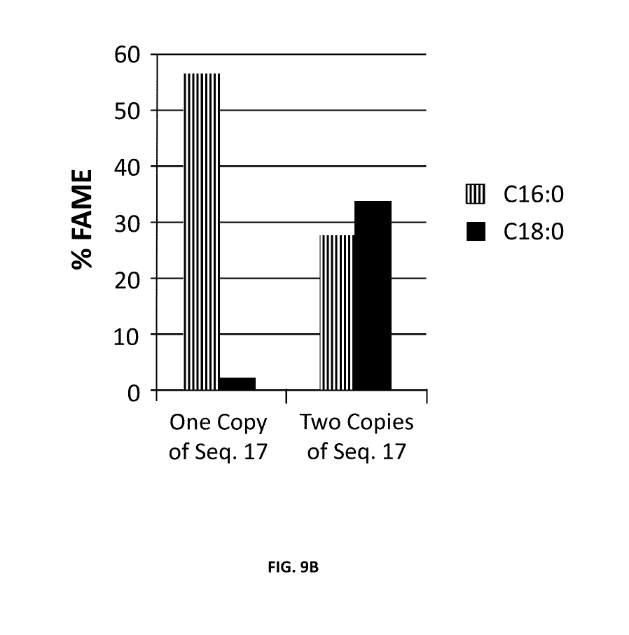

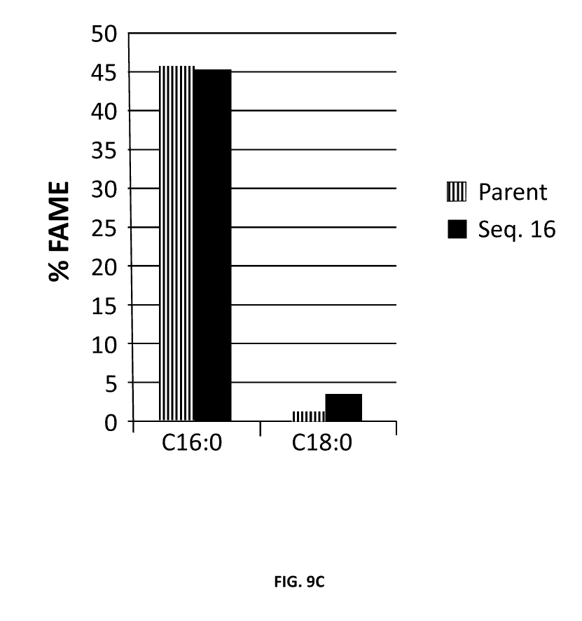

FIGS. 9A, 9B, and 9C provide bar charts showing the expression of the C16 elongases, (9A) SEQ ID NO: 17 in S. cerevisiae and (9B) Aurantiochytrium and (9C) SEQ ID NO: 16 in Aurantiochytrium.

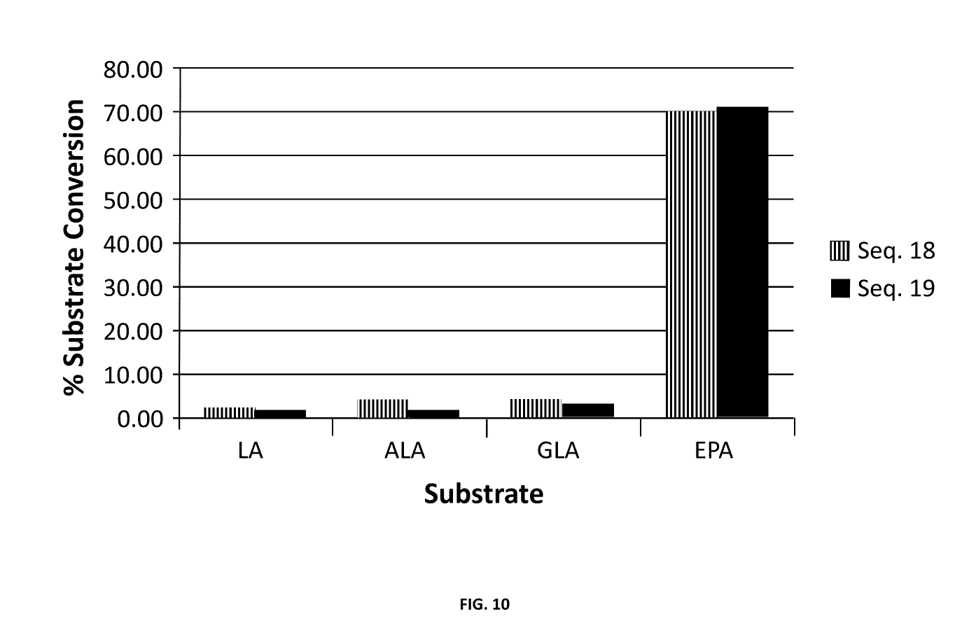

FIG. 10 provides a bar chart showing the activity and specificity of the Δ5 elongases encoded by SEQ ID NOs: 18 and 19 in S. cerevisiae.

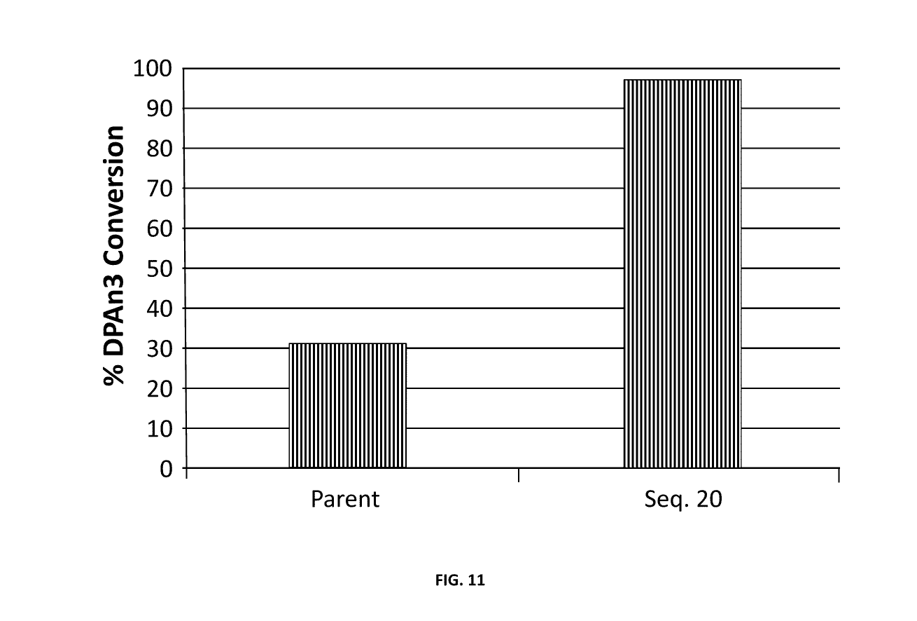

FIG. 11 provides a bar chart showing the activity of the Δ4 elongase encoded by SEQ ID NO: 20 in an Aurantiochytrium PUFA auxotrophic strain.

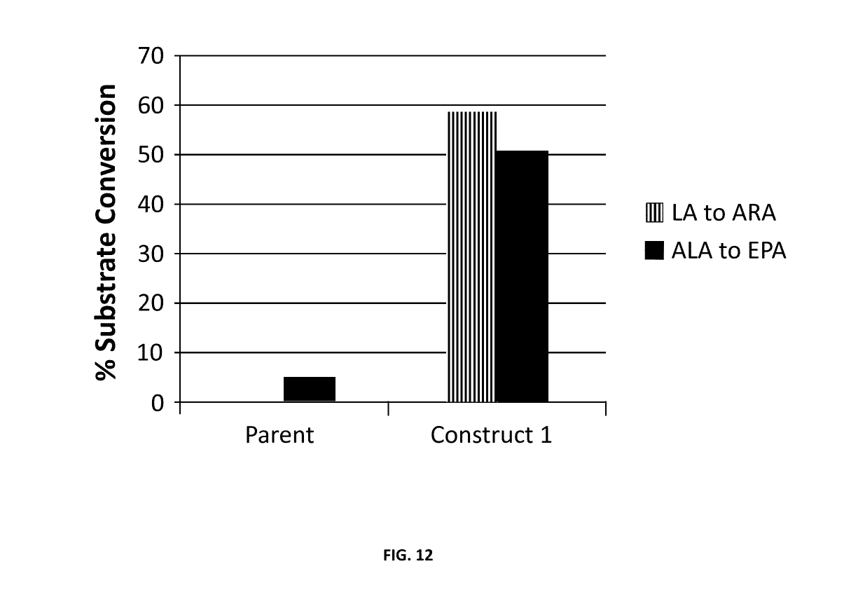

FIG. 12 provides a bar chart showing the expression of Construct 1 (SEQ ID NOs: 2, 6, and 9) in an Aurantiochytrium PUFA auxotrophic strain co-fed DHA and LA or ALA.

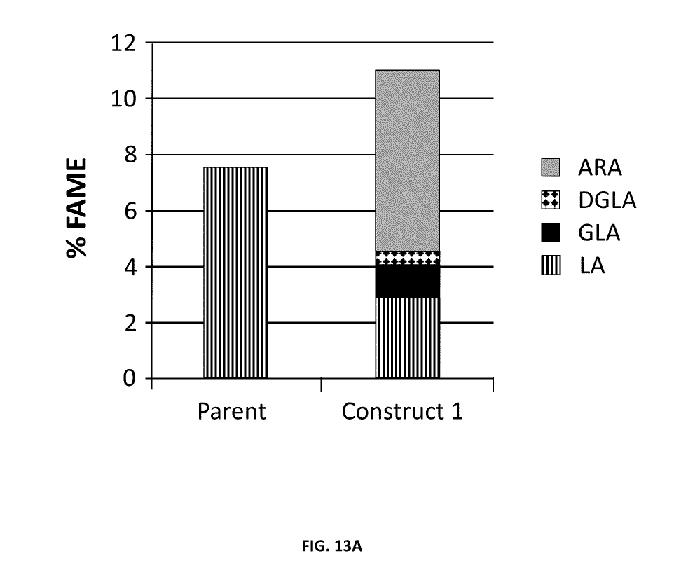

FIGS. 13A and 13B provides bar charts showing the accumulation of pathway intermediates in a strain expressing Construct 1 (SEQ ID NOs: 2, 6, and 9) in an Aurantiochytrium PUFA auxotrophic strain co-fed DHA and (13A) LA or (13B) ALA.

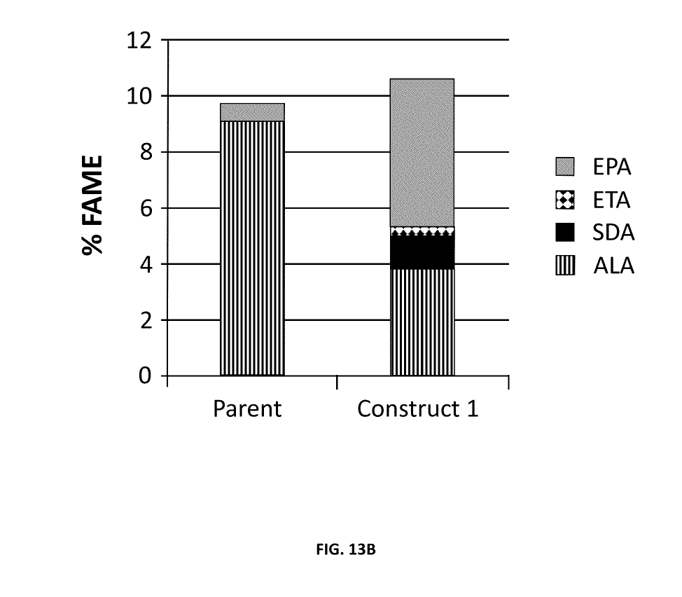

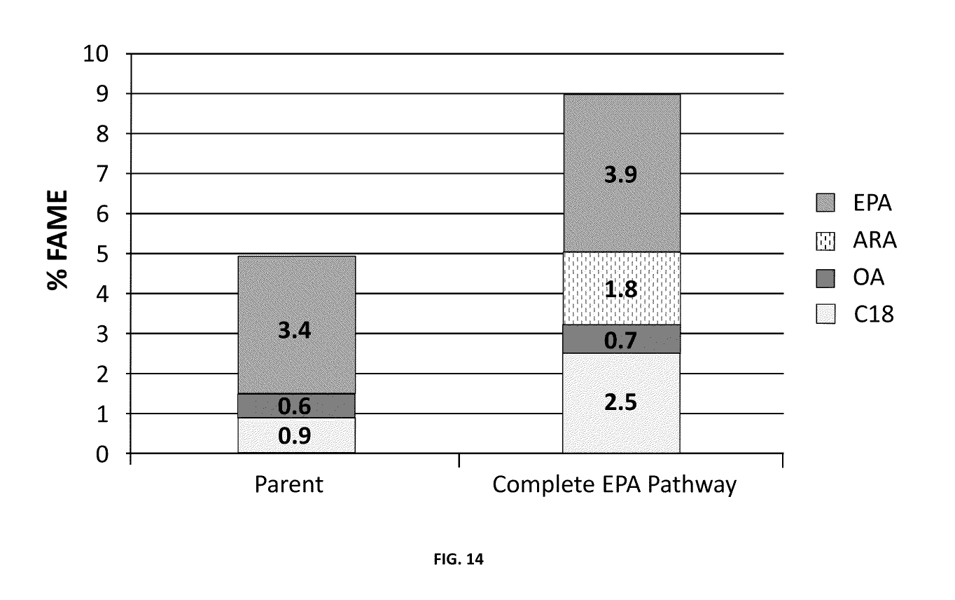

FIG. 14 provides a bar chart showing the expression of the complete C16:0 to EPA elongase/desaturase pathway in an Aurantiochytrium PUFA auxotrophic strain.

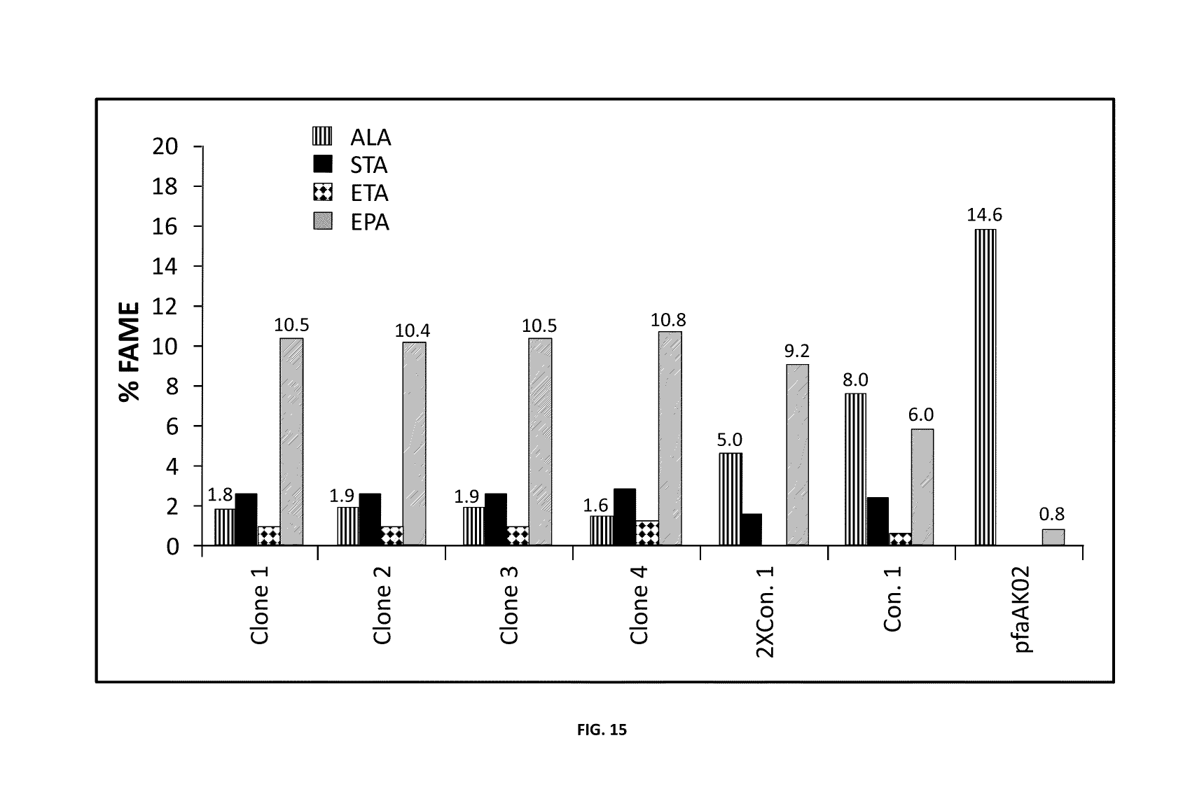

FIG. 15 provides a bar chart showing overexpression of the Δ6 desaturase (SEQ ID NO: 9) with the host's full-length tubulin alpha chain promoter (Tubα-997p) in a strain harboring Construct 1. Four different clones containing an additional copy of the Tubα-997p-driven SEQ ID NO: 9 (clones 1-4), the parent strain containing only Construct 1 (Con. 1), a strain harboring two copies of Construct 1 (2×Con. 1), and an Aurantiochytrium PUFA auxotrophic strain lacking any constructs (pfaAKO2) were fed ALA, and the resulting FAME profiles were analyzed. All of the clones harboring an extra copy of SEQ ID NO: 9 under the control of Tubα-997p exhibited much lower ALA accumulation than the other strains, demonstrating the improved activity of SEQ ID NO: 9.

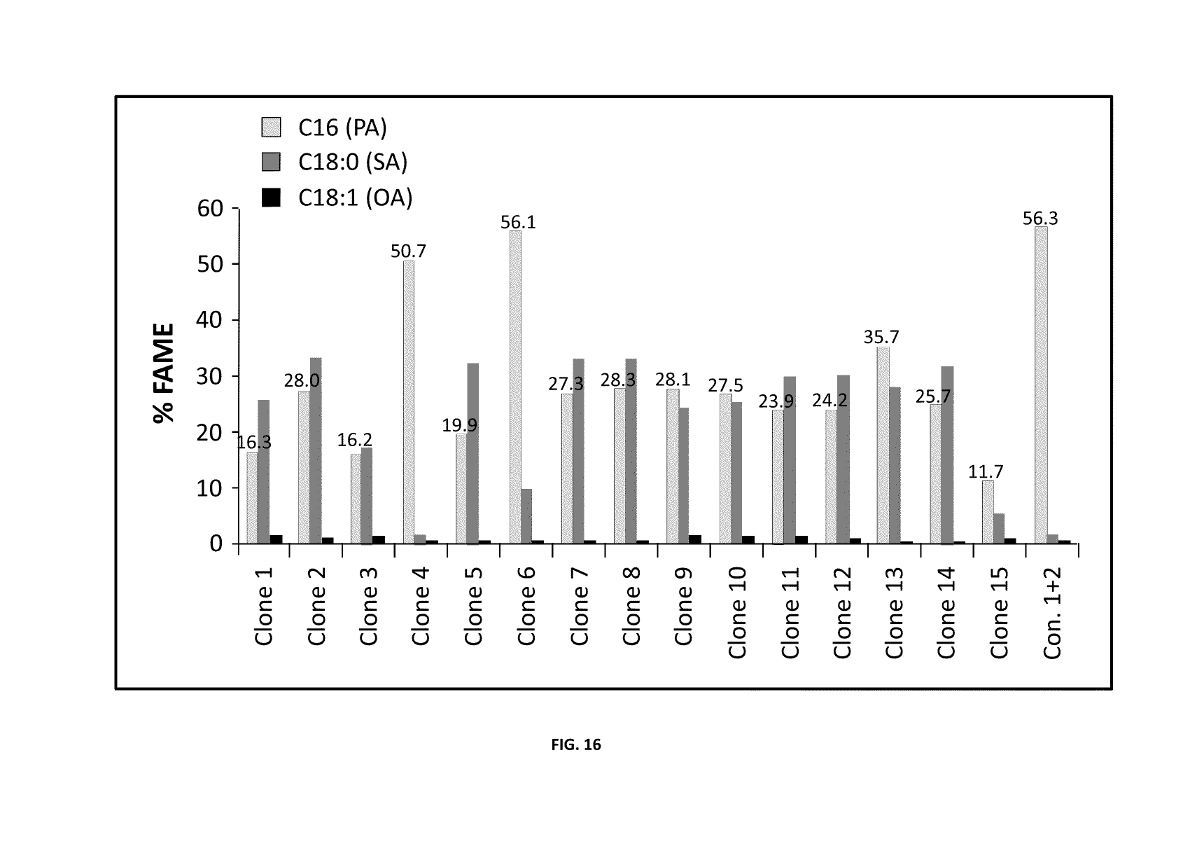

FIG. 16 provides a bar chart showing overexpression of the C16 elongase (SEQ ID NO: 17) with the host's full-length tubulin promoter (Tubα-997p) in a strain harboring Constructs 1 and 2. Con. 1+2 is the parent of the 15 different clones that contain an additional Tubα-997p-driven copy of SEQ ID NO: 17 (clones 1-15). Most clones exhibited a step-change improvement in the conversion of C16:0 to C18:0 when compared to the parent, demonstrating the improved activity of SEQ ID NO: 17.

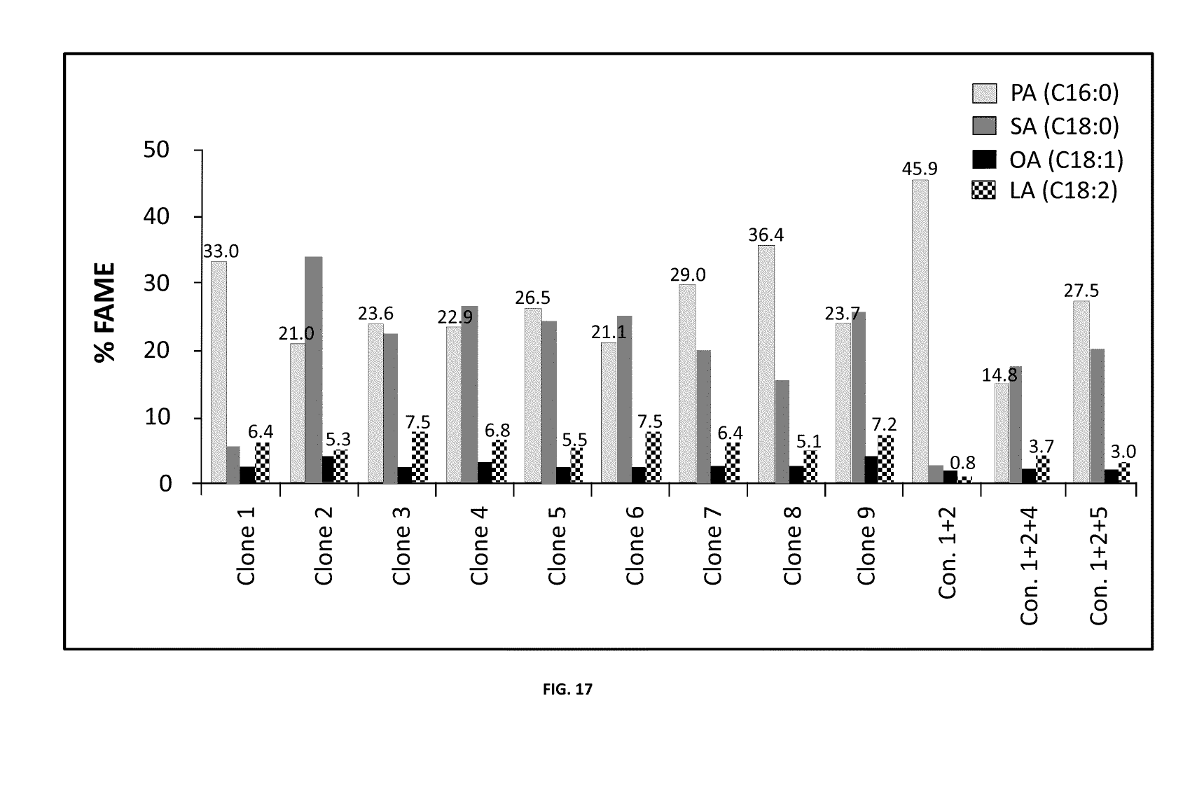

FIG. 17 provides a bar chart showing overexpression of the Δ9 desaturase (SEQ ID NO: 14) with the host's shortened RPL11 promoter (RPL11-699p) in a strain harboring Constructs 1 and 2. Con. 1+2 is the parent of 9 different clones expressing Construct 3 (clones 1-9). Construct 3 harbors an additional copy of SEQ ID NO: 14 driven by RPL11-699p (as well as Seq. 17 driven by Tubα-997p). Construct 4 harbors only SEQ ID NO: 17 driven by Tubα-997p, whereas Construct 5 harbors SEQ ID NO: 17 driven by Tubα-997p and a copy of SEQ ID NO: 14 under the control of the original Tsp-749p. Constructs 4 and 5 were separately transformed into the Con. 1+2 parent, and the resulting strains were used as controls. Higher levels of LA accumulated in clones 1-9 than in the Construct 5 control, demonstrating increased activity of SEQ ID NO: 14 and improved flux at this step of the pathway.

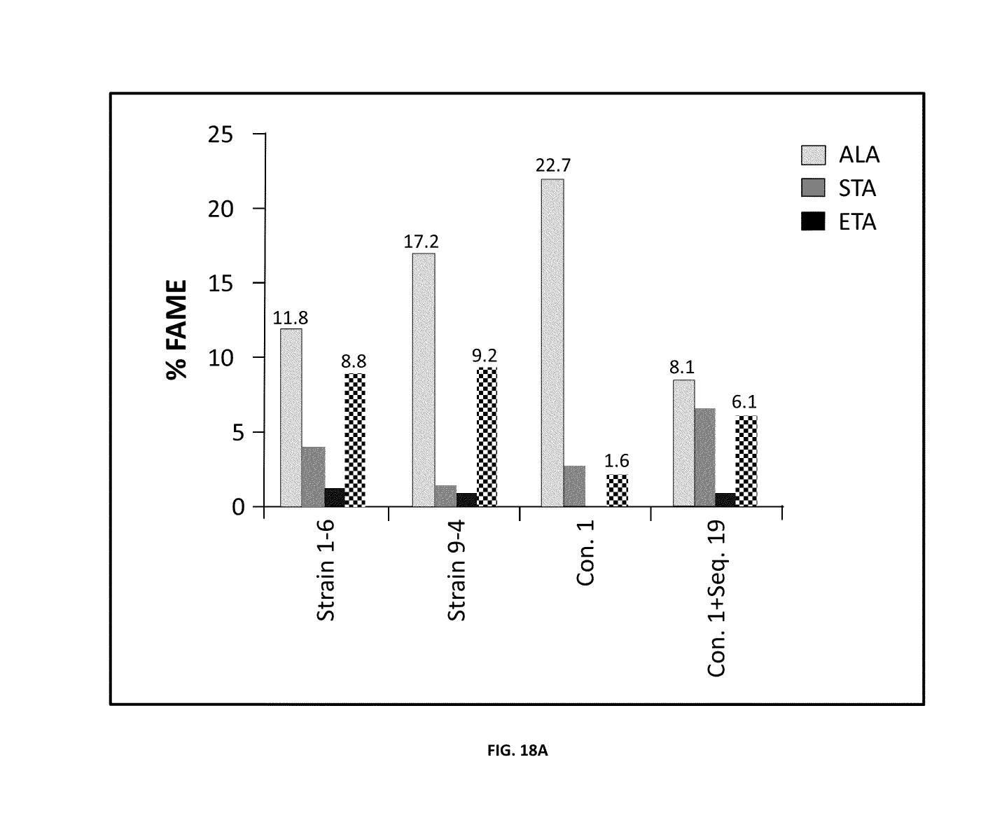

FIGS. 18A and 18B provide bar charts showing expression of the second-generation Constructs 7 and 6. 18A: Strains 1-6 and 9-4 are ΔpfaA/ΔpfaA or ΔpfaB/ΔpfaB Aurantiochytrium PUFA auxotrophic strains, respectively, harboring Construct 7; 18B: strains 6-5 and 12-6 are ΔpfaA/ΔpfaA or ΔpfaB/ΔpfaB Aurantiochytrium PUFA auxotrophic strains, respectively, harboring Construct 6. ΔpfaA/ΔpfaA Aurantiochytrium strains expressing Construct 1 (Con. 1), Construct 1 with an additional copy of SEQ ID NO: 9 (Con. 1+Seq. 9), both Constructs 1 and 2 (Con. 1+2), or Constructs 1, 2, and 3 (Con. 1+2+3) were also included for comparison. All of the strains expressing Construct 6 or 7 had lower levels of substrates and higher levels of final products than control strains harboring the corresponding first-generation constructs. In terms of final product formation, 18A: strains 1-6 and 9-4 outperformed Con. 1+SEQ ID NO: 9, which harbors two copies of SEQ ID NO: 9; 18B: strain 12-6 outperformed Con. 1+2+3, which harbors two copies of SEQ ID NOs: 17 and 19. Strains in 18A were fed ALA prior to FAME analysis.

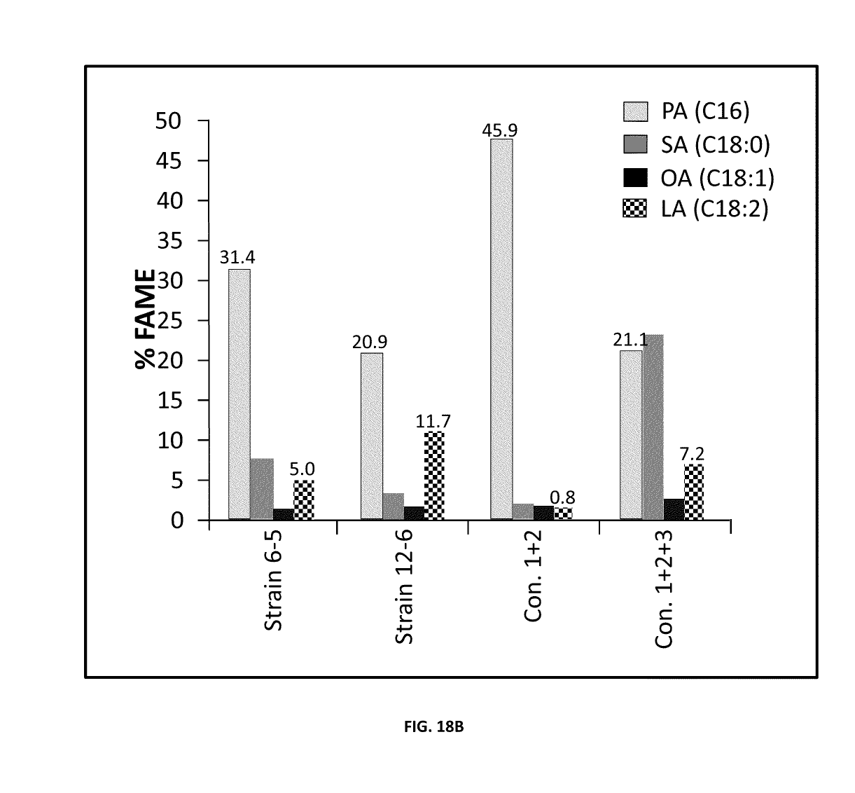

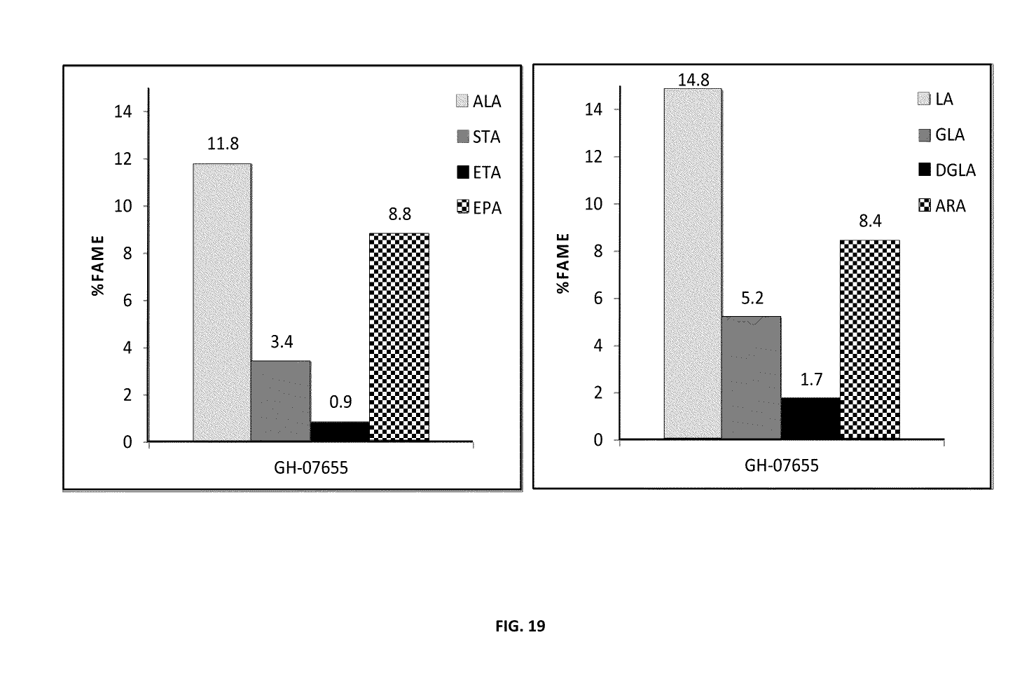

FIG. 19 provides a bar chart illustrating the FAME profiles of GH-07655 after feeding ALA (19A) or LA (19B).

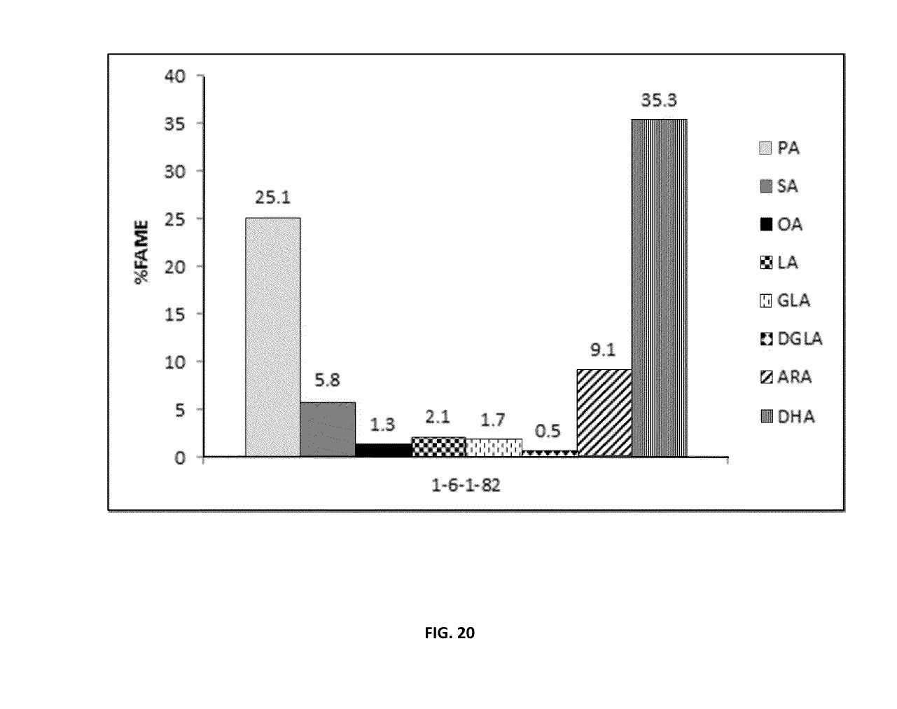

FIG. 20 provides a bar chart illustrating the FAME profile of GH-07917 in FM002 medium containing 1 mM DHA.

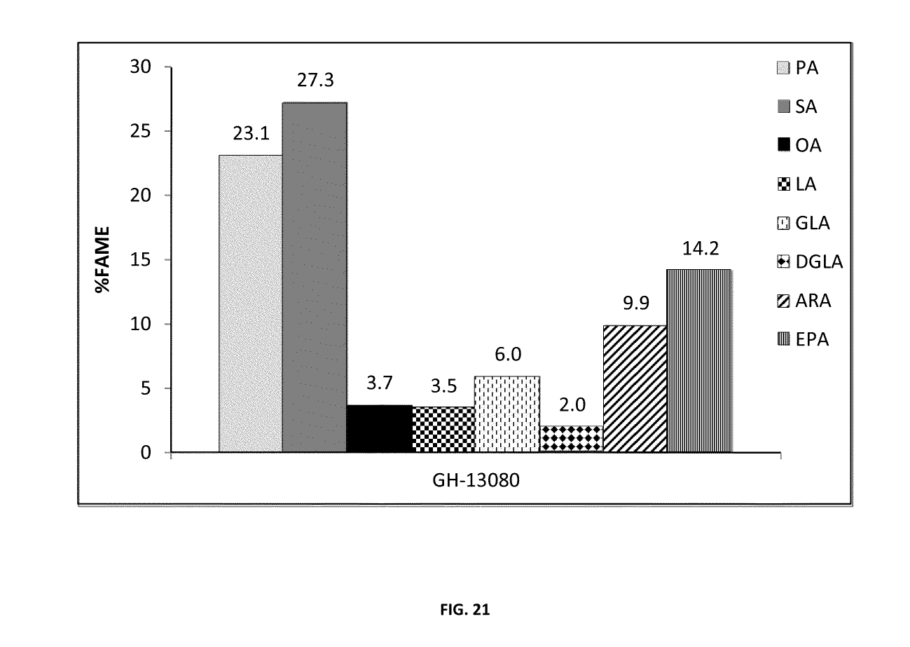

FIG. 21 provides a bar chart illustrating the FAME profile of GH-13080 in medium without PUFA supplementation.

### DETAILED DESCRIPTION OF THE INVENTION

The present invention provides recombinant cells and organisms engineered for the production of a wide variety of lipid molecules, including polyunsaturated fatty acids (PUFAs). The microorganisms can comprise one or more heterologous enzymes, for example at least one heterologous elongase and/or at least one heterologous desaturase. In some embodiments the product of at least one heterologous enzyme is the substrate of another heterologous enzyme and therefore a pathway is engineered into the microorganism for producing one or more polyunsaturated fatty acids (PUFAs). In some embodiments the cell or organism is a Labyrinthulomycetes. Also provided are microbial oils, biomass, and food products and ingredients produced by or comprising the cells or microorganisms of the invention, nucleic acids encoding enzymes used in the substrate to product conversion steps, and methods of use of the same.

The invention provides many advantages over existing methods of producing PUFAs and allows for the creation of a sustainable, low cost, vegetarian source of a wide variety of PUFAs, microbial oils, biomass, human and animal food products and ingredients, pharmaceutical compositions, and other compositions containing the same. The microorganisms of the invention can be engineered to produce a wide variety of PUFAs of choice, e.g., EPA or DHA. Therefore, in various embodiments the compositions and methods can provide separate sources of low-cost individual PUFAs. The invention therefore allows the production of microbial oils and other compositions that contain any desired ratio of specific PUFAs, for example a specific ratio of EPA:DHA. The oils can be produced with high purity and the invention eliminates the need for costly purification procedures. The invention therefore allows for the production of the compositions of the invention that are highly enriched with the PUFA of choice. Furthermore, the compositions and methods of the invention are not dependent on harvesting PUFA-containing compositions from marine life, and therefore the supply is renewable, environmentally friendly, and almost limitless.

### SOME DEFINITIONS

As used herein, the term “construct” is intended to mean any recombinant nucleic acid molecule such as an expression cassette, vector, plasmid, cosmid, virus, autonomously replicating polynucleotide molecule, phage, or linear or circular, single-stranded or double-stranded, DNA or RNA polynucleotide molecule, derived from any source, capable of genomic integration or autonomous replication, comprising a nucleic acid molecule where one or more nucleic acid sequences has been linked in a functionally operative manner, i.e. operably linked.

As used herein, “exogenous” with respect to a nucleic acid or gene indicates that the nucleic or gene has been introduced (“transformed”) into an organism, microorganism, or cell by human intervention. Typically, such an exogenous nucleic acid is introduced into a cell or organism via a recombinant nucleic acid construct. An exogenous nucleic acid can be a sequence from one species introduced into another species, i.e., a heterologous nucleic acid. An exogenous nucleic acid can also be a sequence that is homologous to an organism (i.e., the nucleic acid sequence occurs naturally in that species or encodes a polypeptide that occurs naturally in the host species) that has been isolated and subsequently reintroduced into cells of that organism. An exogenous nucleic acid that includes a homologous sequence can often be distinguished from the naturally-occurring sequence by the presence of non-natural sequences linked to the exogenous nucleic acid, e.g., non-native regulatory sequences flanking the homologous gene sequence in a recombinant nucleic acid construct. Alternatively or in addition, a stably transformed exogenous nucleic acid can be detected and/or distinguished from a native gene by its juxtaposition to sequences in the genome where it has integrated. Further, a nucleic acid is considered exogenous if it has been introduced into a progenitor of the cell, organism, or strain under consideration.

As used herein, “expression” refers to the process of converting genetic information of a polynucleotide into RNA through transcription, which is typically catalyzed by an enzyme, RNA polymerase, and, where the RNA encodes a polypeptide, into protein, through translation of mRNA on ribosomes to produce the encoded protein.

The term “expression cassette” as used herein, refers to a nucleic acid construct that encodes a protein or functional RNA operably linked to expression control elements, such as a promoter, and optionally, any or a combination of other nucleic acid sequences that affect the transcription or translation of the gene, such as, but not limited to, a transcriptional terminator, a ribosome binding site, a splice site or splicing recognition sequence, an intron, an enhancer, a polyadenylation signal, an internal ribosome entry site, etc.

A “fatty acid” is a carboxylic acid with a long aliphatic tail, which can be either saturated or unsaturated. PUFAs are polyunsaturated fatty acids containing two or more double bonds in the aliphatic tail. Most naturally occurring fatty acids have a chain of an even number of carbon atoms, from 4-28, but can also be an even number from 12-22 or from 16-22. Fatty acids are usually derived from triglycerides or phospholipids. Numerous examples of fatty acids are described herein.

A “functional RNA molecule” is an RNA molecule that can interact with one or more proteins or nucleic acid molecules to perform or participate in a structural, catalytic, or regulatory function that affects the expression or activity of a gene or gene product other than the gene that produced the functional RNA. A functional RNA can be, for example, a messenger RNA (mRNA), a transfer RNA (tRNA), ribosomal RNA (rRNA), antisense RNA (asRNA), microRNA (miRNA), short hairpin RNA (shRNA), small interfering RNA (siRNA), small nucleolar RNAs (snoRNAs), piwi-interacting RNA (piRNA), or a ribozyme.

The term “gene” is used broadly to refer to any segment of nucleic acid molecule that encodes a protein or that can be transcribed into a functional RNA. Genes may include sequences that are transcribed but are not part of a final, mature, and/or functional RNA transcript, and genes that encode proteins may further comprise sequences that are transcribed but not translated, for example, 5′ untranslated regions, 3′ untranslated regions, introns, etc. Further, genes may optionally further comprise regulatory sequences required for their expression, and such sequences may be, for example, sequences that are not transcribed or translated. Genes can be obtained from a variety of sources, including cloning from a source of interest or synthesizing from known or predicted sequence information, and may include sequences designed to have desired parameters.

The term “heterologous” when used in reference to a polynucleotide, a gene, a nucleic acid, a polypeptide, or an enzyme, refers to a polynucleotide, gene, a nucleic acid, polypeptide, or an enzyme that is not derived from the host species. For example, “heterologous gene” or “heterologous nucleic acid sequence” as used herein, refers to a gene or nucleic acid sequence from a different species than the species of the host organism it is introduced into. When referring to a gene regulatory sequence or to an auxiliary nucleic acid sequence used for manipulating expression of a gene sequence (e.g. a 5′ untranslated region, 3′ untranslated region, poly A addition sequence, intron sequence, splice site, ribosome binding site, internal ribosome entry sequence, genome homology region, recombination site, etc.) or to a nucleic acid sequence encoding a protein domain or protein localization sequence, “heterologous” means that the regulatory or auxiliary sequence or sequence encoding a protein domain or localization sequence is from a different source than the gene with which the regulatory or auxiliary nucleic acid sequence or nucleic acid sequence encoding a protein domain or localization sequence is juxtaposed in a genome, chromosome or episome. Thus, a promoter operably linked to a gene to which it is not operably linked to in its natural state (for example, in the genome of a non-genetically engineered organism) is referred to herein as a “heterologous promoter,” even though the promoter may be derived from the same species (or, in some cases, the same organism) as the gene to which it is linked. Similarly, when referring to a protein localization sequence or protein domain of an engineered protein, “heterologous” means that the localization sequence or protein domain is derived from a protein different from that into which it is incorporated by genetic engineering.

The term “native” is used herein to refer to nucleic acid sequences or amino acid sequences as they naturally occur in the host. The term “non-native” is used herein to refer to nucleic acid sequences or amino acid sequences that do not occur naturally in the host, or are not configured as they are naturally configured in the host. A nucleic acid sequence or amino acid sequence that has been removed from a host cell, subjected to laboratory manipulation, and introduced or reintroduced into a host cell is considered “non-native.” Synthetic or partially synthetic genes introduced into a host cell are “non-native.” Non-native genes further include genes endogenous to the host microorganism operably linked to one or more heterologous regulatory sequences that have been recombined into the host genome, or genes endogenous to the host organism that is in a locus of the genome other than that where they naturally occur.

The terms “naturally-occurring” and “wild-type”, as used herein, refer to a form found in nature. For example, a naturally occurring or wild-type nucleic acid molecule, nucleotide sequence or protein may be present in and isolated from a natural source, and is not intentionally modified by human manipulation.

The terms “nucleic acid molecule” and “polynucleotide” are used interchangeably herein, and refer to both RNA and DNA molecules, including nucleic acid molecules comprising cDNA, genomic DNA, synthetic DNA, and DNA or RNA molecules containing nucleic acid analogs. Nucleic acid molecules can have any three-dimensional structure. A nucleic acid molecule can be double-stranded or single-stranded (e.g., a sense strand or an antisense strand). Non-limiting examples of nucleic acid molecules include genes, gene fragments, exons, introns, messenger RNA (mRNA), transfer RNA, ribosomal RNA, siRNA, micro-RNA, tracrRNAs, crRNAs, guide RNAs, ribozymes, cDNA, recombinant polynucleotides, branched polynucleotides, nucleic acid probes and nucleic acid primers. A nucleic acid molecule may contain unconventional or modified nucleotides. The terms “polynucleotide sequence” and “nucleic acid sequence” as used herein interchangeably refer to the sequence of a polynucleotide molecule. The nomenclature for nucleotide bases as set forth in 37 CFR §1.822 is used herein.

The nucleic acid molecules of the present disclosure will preferably be “biologically active” with respect to either a structural attribute, such as the capacity of a nucleic acid molecule to hybridize to another nucleic acid molecule, or the ability to a nucleic acid sequence to be recognized and bound by a transcription factor (or to compete with another nucleic acid molecule for such binding).

Nucleic acid molecules of the present disclosure will include nucleic acid sequences of any length, including nucleic acid molecules that are preferably between about 0.05 Kb and about 300 Kb, for example between about 0.05 Kb and about 250 Kb, between about 0.05 Kb and about 150 Kb, or between about 0.1 Kb and about 150 Kb, for example between about 0.2 Kb and about 150 Kb, about 0.5 Kb and about 150 Kb, or about 1 Kb and about 150 Kb.

The term “operably linked”, as used herein, denotes a functional linkage between two or more sequences. For example, an operable linkage between a polynucleotide of interest and a regulatory sequence (for example, a promoter) is a functional link that allows for expression of the polynucleotide of interest. In this sense, the term “operably linked” refers to the positioning of a regulatory region and a coding sequence to be transcribed so that the regulatory region is effective for regulating transcription or translation of the coding sequence of interest. In some embodiments disclosed herein, the term “operably linked” denotes a configuration in which a regulatory sequence is placed at an appropriate position relative to a sequence that encodes a polypeptide or functional RNA such that the control sequence directs or regulates the expression or cellular localization of the mRNA encoding the polypeptide, the polypeptide, and/or the functional RNA. Thus, a promoter is in operable linkage with a nucleic acid sequence if it can mediate transcription of the nucleic acid sequence. Operably linked elements may be contiguous or non-contiguous. Further, when used to refer to the joining of two protein coding regions, by “operably linked” is intended that the coding regions are in the same reading frame.

The terms “promoter”, “promoter region”, or “promoter sequence” refer to a nucleic acid sequence capable of binding RNA polymerase to initiate transcription of a gene in a 5′ to 3′ (“downstream”) direction. A gene is “under the control of” or “regulated by” a promoter when the binding of RNA polymerase to the promoter is the proximate cause of said gene's transcription. The promoter or promoter region typically provides a recognition site for RNA polymerase and other factors necessary for proper initiation of transcription. A promoter may be isolated from the 5′ untranslated region (5′ UTR) of a genomic copy of a gene. Alternatively, a promoter may be synthetically produced or designed by altering known DNA elements. Also considered are chimeric promoters that combine sequences of one promoter with sequences of another promoter. Promoters may be defined by their expression pattern based on, for example, metabolic, environmental, or developmental conditions. A promoter can be used as a regulatory element for modulating expression of an operably linked polynucleotide molecule such as, for example, a coding sequence of a polypeptide or a functional RNA sequence. Promoters may contain, in addition to sequences recognized by RNA polymerase and, preferably, other transcription factors, regulatory sequence elements such as cis-elements or enhancer domains that affect the transcription of operably linked genes. A “Labyrinthulomycetes promoter” as used herein refers to a native or non-native promoter that is functional in labyrinthulomycetes cells.

The term “recombinant” or “engineered” nucleic acid molecule as used herein, refers to a nucleic acid molecule that has been altered through human intervention. As non-limiting examples, a cDNA is a recombinant DNA molecule, as is any nucleic acid molecule that has been generated by in vitro polymerase reaction(s), or to which linkers have been attached, or that has been integrated into a vector, such as a cloning vector or expression vector. As non-limiting examples, a recombinant nucleic acid molecule: 1) has been synthesized or modified in vitro, for example, using chemical or enzymatic techniques (for example, by use of chemical nucleic acid synthesis, or by use of enzymes for the replication, polymerization, exonucleolytic digestion, endonucleolytic digestion, ligation, reverse transcription, transcription, base modification (including, e.g., methylation), or recombination (including homologous and site-specific recombination)) of nucleic acid molecules; 2) includes conjoined nucleotide sequences that are not conjoined in nature, 3) has been engineered using molecular cloning techniques such that it lacks one or more nucleotides with respect to the naturally occurring nucleic acid molecule sequence, and/or 4) has been manipulated using molecular cloning techniques such that it has one or more sequence changes or rearrangements with respect to the naturally occurring nucleic acid sequence. As non-limiting examples, a cDNA is a recombinant DNA molecule, as is any nucleic acid molecule that has been generated by in vitro polymerase reaction(s), or to which linkers have been attached, or that has been integrated into a vector, such as a cloning vector or expression vector.

When applied to organisms, the terms “transgenic” “transformed” or “recombinant” or “engineered” or “genetically engineered” refer to organisms that have been manipulated by the introduction of an exogenous or recombinant nucleic acid sequence into the organism. Non-limiting examples of such manipulations include gene knockouts, targeted mutations and gene replacement, promoter replacement, deletion, or insertion, as well as the introduction of transgenes into the organism. For example, a transgenic microorganism can include an introduced exogenous regulatory sequence operably linked to an endogenous gene of the transgenic microorganism. Recombinant or genetically-engineered organisms can also be organisms into which constructs for gene “knock-down” have been introduced. Such constructs include, but are not limited to, RNAi, microRNA, shRNA, antisense, and ribozyme constructs. Also included are organisms whose genomes have been altered by the activity of meganucleases or zinc finger nucleases. A heterologous or recombinant nucleic acid molecule can be integrated into a genetically engineered/recombinant organism's genome or, in other instances, not integrated into a recombinant/genetically engineered organism's genome. As used herein, “recombinant microorganism” or “recombinant host cell” includes progeny or derivatives of the recombinant microorganisms of the disclosure. Because certain modifications may occur in succeeding generations from either mutation or environmental influences, such progeny or derivatives may not, in fact, be identical to the parent cell, but are still included within the scope of the term as used herein.

“Regulatory sequence”, “regulatory element”, or “regulatory element sequence” refers to a nucleotide sequence located upstream (5′), within, or downstream (3′) of a polypeptide-encoding sequence or functional RNA-encoding sequence. Transcription of the polypeptide-encoding sequence or functional RNA-encoding sequence and/or translation of an RNA molecule resulting from transcription of the coding sequence are typically affected by the presence or absence of the regulatory sequence. These regulatory element sequences may comprise promoters, cis-elements, enhancers, terminators, or introns. Regulatory elements may be isolated or identified from untranslated regions (UTRs) from a particular polynucleotide sequence. Any of the regulatory elements described herein may be present in a chimeric or hybrid regulatory expression element. Any of the regulatory elements described herein may be present in a recombinant construct of the present disclosure.

The term “terminator” or “terminator sequence” or “transcription terminator”, as used herein, refers to a regulatory section of genetic sequence that causes RNA polymerase to cease transcription.

The term “transformation”, “transfection”, and “transduction”, as used interchangeably herein, refers to the introduction of one or more exogenous nucleic acid sequences into a host cell or organism by using one or more physical, chemical, or biological methods. Physical and chemical methods of transformation include, by way of non-limiting example, electroporation and liposome delivery. Biological methods of transformation include transfer of DNA using engineered viruses or microbes (for example, Agrobacterium).

As used herein, the term “vector” refers to a recombinant polynucleotide construct designed for transfer between host cells, and that may be used for the purpose of transformation, i.e. the introduction of heterologous DNA into a host cell. As such, the term “vector” as used herein sometimes refers to a replicon, such as a plasmid, phage, or cosmid, into which another DNA segment may be inserted so as to bring about the replication of the inserted segment. A vector typically includes one or both of 1) an origin of replication, and 2) a selectable marker. A vector can additionally include sequence for mediating recombination of a sequence on the vector into a target genome, cloning sites, and/or regulatory sequences such as promoters and/or terminators. Generally, a vector is capable of replication when associated with the proper control elements. The term “vector” includes cloning vectors and expression vectors, as well as viral vectors and integrating vectors. An “expression vector” is a vector that includes a regulatory region, thereby capable of expressing DNA sequences and fragments in vitro and/or in vivo.

The cells or organisms of the invention can be any microorganism of the class Labyrinthulomycetes. While the classification of the Thraustochytrids and Labyrinthulids has evolved over the years, for the purposes of the present application, “labyrinthulomycetes” is a comprehensive term that includes microorganisms of the orders Thraustochytrid and Labyrinthulid, and includes (without limitation) the genera Althornia, Aplanochytrium, Aurantiochytrium, Corallochytrium, Diplophryids, Diplophrys, Elina, Japonochytrium, Labyrinthula, Labryinthuloides, Oblongichytrium, Pyrrhosorus, Schizochytrium, Thraustochytrium, and Ulkenia. In some examples the microorganism is from a genus including, but not limited to, Thraustochytrium, Labyrinthuloides, Japonochytrium, and Schizochytrium. Alternatively, a host labyrinthulomycetes microorganism can be from a genus including, but not limited to Aurantiochytrium, Oblongichytrium, and Ulkenia. Examples of suitable microbial species within the genera include, but are not limited to: any Schizochytrium species, including Schizochytrium aggregatum, Schizochytrium limacinum, Schizochytrium minutum; any Thraustochytrium species (including former Ulkenia species such as U. visurgensis, U. amoeboida, U. sarkariana, U. profunda, U. radiata, U. minuta and Ulkenia sp. BP-5601), and including Thraustochytrium striatum, Thraustochytrium aureum, Thraustochytrium roseum; and any Japonochytrium species. Strains of Thraustochytriales particularly suitable for the presently disclosed invention include, but are not limited to: Schizochytrium sp. (S31) (ATCC 20888); Schizochytrium sp. (S8) (ATCC 20889); Schizochytrium sp. (LC-RM) (ATCC 18915); Schizochytrium sp. (SR21); Schizochytrium aggregatum (ATCC 28209); Schizochytrium limacinum (IFO 32693); Thraustochytrium sp. 23B ATCC 20891; Thraustochytrium striatum ATCC 24473; Thraustochytrium aureum ATCC 34304); Thraustochytrium roseum (ATCC 28210; and Japonochytrium sp. Ll ATCC 28207.

The PKS and Elo/Des Pathways

In organisms of the class Labyrinthulomycetes fatty acids can be synthesized or altered by an elongase/desaturase biosynthetic pathway (the “elo/des pathway”), which utilizes the actions of a) desaturases that introduce double bonds in the aliphatic chain of a fatty acid, and by the actions of b) elongases, which extend the acyl chain by two carbon units. However, in many organisms (e.g., marine bacteria and certain eukaryotes such as some members of the Labyrinthulomycetes) fatty acids are synthesized via a polyketide synthase pathway (PKS). The polyketide synthases (PKSs) are a family of multi-domain enzyme complexes that produce various polyketides. The recombinant organisms of the invention can contain one or more of the pathways, chains, networks, or substrate to product conversion steps as described herein, which can be present as exogenous pathways, chains, or networks. In one embodiment the recombinant cells and organisms of the invention comprise an exogenous elo/des pathway or portion thereof engineered into the cell or organism that does not naturally have such pathway. In some embodiments the cells or organisms of the invention have an exogenous PKS pathway or portion thereof. The cells or organisms of the invention can also have a native PKS pathway that has been disrupted, deleted, or impaired. Disruption refers to a change in the pathway such that the cell or organism cannot use the PKS pathway to convert certain products of primary metabolism (such as acetyl-CoA and malonyl-CoA) into DHA. Deletion of all or part of the pathway is one method of disruption. Impairment means the cell or organism can use the pathway but it produces a reduced amount of DHA due to an inefficiency introduced in the pathway. The PKS pathway can be disrupted or “knocked out” by inserting DNA into the pfaA, pfaB, or pfaC alleles, or a partial or full deletion of the pfaA, pfaB, or pfaC alleles, and in some embodiments both alleles of pfaA and/or pfaB are deleted. The PKS pathway can be impaired by attenuating expression of the pfaA, B, or C genes modifying the promoters, using RNAi or other methods of attenuating gene expression. In some embodiments the flux of the pathway is improved by the engineering disclosed herein.

For example, gene knockout or replacement by homologous recombination can be by transformation of a nucleic acid (e.g., DNA) fragment that includes a sequence homologous to the region of the genome to be altered, where the homologous sequence is interrupted by a heterologous sequence, typically a selectable marker gene that allows selection for the integrated construct. The genome-homologous flanking sequences on either side of the foreign sequence or mutated gene sequence can be for example, at least 50, at least 100, at least 200, at least 300, at least 400, at least 500, at least 600, at least 700, at least 800, at least 900, at least 1,000, at least 1,200, at least 1,500, at least 1,750, or at least 2,000 nucleotides in length. A gene knockout or gene “knock in” construct, in which a foreign sequence is flanked by target gene sequences, can be provided in a vector that can optionally be linearized, for example, outside of the region that is to undergo homologous recombination, or can be provided as a linear fragment that is not in the context of a vector, for example, the knock-out or knock-in construct can be an isolated or synthesized fragment, including but not limited to a PCR product. In some instances, a split marker system can be used to generate gene knock-outs by homologous recombination, where two DNA fragments can be introduced that can regenerate a selectable marker and disrupt the gene locus of interest via three crossover events (Jeong et al. (2007) FEMS Microbiol Lett 273: 157-163).

The disrupted gene can be disrupted by, for example, an insertion, mutation, or gene replacement mediated by homologous recombination and/or by the activity of a meganuclease, zinc finger nuclease (Perez-Pinera et al. (2012) Curr. Opin. Chem. Biol. 16: 268-277), TALEN, or a cas protein (e.g., a cas9 protein) of a CRISPR system.

CRISPR systems, reviewed recently by Hsu et al. (Cell 157:1262-1278, 2014) include, in addition to the cas nuclease polypeptide or complex, a targeting RNA, often denoted “crRNA”, that interacts with the genome target site by complementarity with a target site sequence, a trans-activating (“tracr”) RNA that complexes with the cas polypeptide and also includes a region that binds (by complementarity) the targeting crRNA.

The invention contemplates the use of two RNA molecules (“crRNA” and “tracrRNA”) that can be co-transformed into a host strain (or expressed in a host strain) that expresses or is transfected with a cas protein for genome editing, or the use of a single guide RNA that includes a sequence complementary to a target sequence as well as a sequence that interacts with a cas protein. That is, a CRISPR system as used herein can comprise two separate RNA molecules (RNA polynucleotides: a “tracr-RNA” and a “targeter-RNA” or “crRNA”, see below) and referred to herein as a “double-molecule DNA-targeting RNA” or a “two-molecule DNA-targeting RNA.” Alternatively, as illustrated in the examples, the DNA-targeting RNA can also include the trans-activating sequence for interaction with the cas protein in addition to the target-homologous (“cr”) sequences, that is, the DNA-targeting RNA can be a single RNA molecule (single RNA polynucleotide) and is referred to herein as a “chimeric guide RNA,” a “single-guide RNA,” or an “sgRNA.” The terms “DNA-targeting RNA” and “gRNA” are inclusive, referring both to double-molecule DNA-targeting RNAs and to single-molecule DNA-targeting RNAs (i.e sgRNAs). Both single-molecule guide RNAs and two RNA systems have been described in detail in the literature and for example, in US20140068797, incorporated by reference herein.

Any cas protein can be used in the methods herein, e.g., Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. The cas protein can be a cas9 protein, such as a cas9 protein of S. pyogenes, S. thermophilus, S. pneumonia, or Neisseria meningitidis, as nonlimiting examples. Also considered are the cas9 proteins provided as SEQ ID NOs:1-256 and 795-1346 in US20140068797, and chimeric cas9 proteins that may combine domains from more than one cas9 protein, as well variants and mutants of identified cas9 proteins. The cas protein can be expressed in the cell, for example, by transforming the host cell with an expression construct that encodes the cas gene.

Cas nuclease activity cleaves target DNA to produce double strand breaks. These breaks are then repaired by the cell in one of two ways: non-homologous end joining or homology-directed repair. In non-homologous end joining (NHEJ), the double-strand breaks are repaired by direct ligation of the break ends to one another. In this case, no new nucleic acid material is inserted into the site, although some nucleic acid material may be lost, resulting in a deletion, or altered, often resulting in mutation. In homology-directed repair, a donor polynucleotide (sometimes referred to as a “donor DNA” or “editing DNA”) with homology to the cleaved target DNA sequence is used as a template for repair of the cleaved target DNA sequence, resulting in the transfer of genetic information from the donor polynucleotide to the target DNA. As such, new nucleic acid material may be inserted/copied into the site. The modifications of the target DNA due to NHEJ and/or homology-directed repair (for example using a donor DNA molecule) can lead to, for example, gene correction, gene replacement, gene tagging, transgene insertion, nucleotide deletion, gene disruption, gene mutation, etc.

In some instances, cleavage of DNA by a site-directed modifying polypeptide (e.g., a cas nuclease, zinc finger nuclease, meganuclease, or TALEN) may be used to delete nucleic acid material from a target DNA sequence by cleaving the target DNA sequence and allowing the cell to repair the sequence in the absence of an exogenously provided donor polynucleotide. Such NHEJ events can result in mutations (“mis-repair”) at the site of rejoining of the cleaved ends that can resulting in gene disruption.

Alternatively, if a DNA-targeting RNA is co-administered to cells that express a cas nuclease along with a donor DNA, the subject methods may be used to add, i.e. insert or replace, nucleic acid material to a target DNA sequence (e.g. “knock out” by insertional mutagenesis, or “knock in” a nucleic acid that encodes a protein (e.g., a selectable marker and/or any protein of interest), an siRNA, an miRNA, etc., to modify a nucleic acid sequence (e.g., introduce a mutation), and the like.

In some cases, a cas polypeptide such as a Cas9 polypeptide is a fusion polypeptide, comprising, e.g.: i) a Cas9 polypeptide (which can optionally be variant Cas9 polypeptide as described above); and b) a covalently linked heterologous polypeptide (also referred to as a “fusion partner”). A heterologous nucleic acid sequence may be linked to another nucleic acid sequence (e.g., by genetic engineering) to generate a chimeric nucleotide sequence encoding a chimeric polypeptide. In some embodiments, a Cas9 fusion polypeptide is generated by fusing a Cas9 polypeptide with a heterologous sequence that provides for subcellular localization (i.e., the heterologous sequence is a subcellular localization sequence, e.g., a nuclear localization signal (NLS) for targeting to the nucleus; a mitochondrial localization signal for targeting to the mitochondria; a chloroplast localization signal for targeting to a chloroplast; an ER retention signal; and the like). In some embodiments, the heterologous sequence can provide a tag (i.e., the heterologous sequence is a detectable label) for ease of tracking and/or purification (e.g., a fluorescent protein, e.g., green fluorescent protein (GFP), YFP, RFP, CFP, mCherry, tdTomato, and the like; a hemagglutinin (HA) tag; a FLAG tag; a Myc tag; and the like).

Host cells can be genetically engineered (e.g. transduced or transformed or transfected) with, for example, a vector construct that can be, for example, a vector for homologous recombination that includes nucleic acid sequences homologous to a portion of a [X] locus of the host cell or to regions adjacent thereto, or can be an expression vector for the expression of any or a combination of: a cas protein (e.g., a cas9 protein), a CRISPR chimeric guide RNA, a crRNA, and/or a tracrRNA, an RNAi construct (e.g., a shRNA), an antisense RNA, or a ribozyme. The vector can be, for example, in the form of a plasmid, a viral particle, a phage, etc. A vector for expression of a polypeptide or RNA for genome editing can also be designed for integration into the host, e.g., by homologous recombination. A vector containing a polynucleotide sequence as described herein, e.g., sequences having homology to host sequences, as well as, optionally, a selectable marker or reporter gene, can be employed to transform an appropriate host to cause attenuation of a gene.

Any of the nucleic acid sequences and/or amino acid sequences disclosed herein can also have at least one substitution modification versus the disclosed nucleic acid sequence or amino acid sequence. Non-limiting examples of a substitution modification include a substitution, an insertion, a deletion, a rearrangement, an inversion, a replacement, a point mutation, and a suppressor mutation. Methods of performing substitution modifications are known in the art and are readily available to the artisan such as, for example, site-specific mutagenesis, PCR, and gene synthesis. Non-limiting examples of substitution modification methods can also be found in Maniatis et al., (1982) Molecular Cloning: a Laboratory Manual; Cold Spring Harbor Laboratory, Cold Spring Harbor, N.Y. In some embodiments the substitution modification(s) do not substantially alter the functional properties of the resulting nucleic acid or amino acid sequence (or fragment thereof) relative to the initial, unmodified fragment, but in other embodiments the substitution modification improves the functional properties. It is therefore understood, as those skilled in the art will appreciate, that the disclosure encompasses more than the specific exemplary sequences. A substitution modification can also include alterations that produce silent substitutions, insertions, deletions, etc. as above, but do not alter the properties or activities of the encoded protein or how the proteins are made.

Recombinant Cells or Organisms

In various embodiments the recombinant cells or organisms of the invention are members of the class Labyrinthulomycetes and can be any described herein. With respect to PUFA production, these organisms predominantly produce DHA. Some Labyrinthulomycetes species, such as those of the genus Aurantiochytrium, use only the PKS system to make DHA while others use the elongase/desaturase pathway, and some use both the PKS and elongase/desaturase pathways.

The elongase/desaturase pathway is generally depicted in FIG. 1, which illustrates various reactions in the pathway or network of enzymatic or chemical reactions to arrive at various fatty acids and intermediates in the pathway or network. In one embodiment of the invention a recombinant organism of the invention produces one or more fatty acids or PUFAs through the action of at least one heterologous elongase and at least one heterologous desaturase, which are functionally expressed in the recombinant organism. An enzyme is functionally expressed when it is expressed at a detectable level (e.g., a substrate to product conversion of 0.5 ug/ml culture volume) and its biological activity is maintained. A large number of enzymes can participate in the elongase/desaturase pathway. Expression of a heterologous enzyme can be from a construct such as a plasmid, or another nucleic acid vector or by integration into the native genome.

The recombinant cells or organisms of the invention can be used to produce a wide variety of useful products such as, for example, microbial oils and microbial biomass containing advantageous amounts and/or ratios of various desired PUFAs.

In some embodiments the cells or organisms of the invention are produced by engineering heterologous elongases and/or desaturases for functional expression in organisms that already have high lipid productivities. The elongases and/or desaturases can be any described herein, which can be expressed as exogenous nucleic acids in the cells or organisms. In the invention such cells or organisms are engineered to have an even more superior capacity to make and store lipids. The microbial oils and biomass of the invention can therefore be produced at low cost and high purity, thereby reducing or eliminating the costs of purification. In some embodiments the cells or organisms of the invention can produce high purity EPA or any other PUFA described herein. In one embodiment the invention therefore eliminates the need to purify EPA from fish oils or other natural sources, resulting in a high purity, low cost source of EPA or any desired PUPA described herein. An additional advantage over oils purified from fish and other marine sources is that the microbial oils of the present invention are provided without concerns about contamination with heavy metals, which is frequently found in natural sources. Yet another advantage of the invention is that the microbial oils and biomass is provided from a vegetarian and environmentally friendly source, thus alleviating concerns with respect to those issues.

Conversion Steps

The following is a non-limiting list of substrate to product conversion steps in a pathway, chain, or network that can be present in a recombinant organism of the invention and one or more of the steps can be performed by a heterologous enzyme. Any of the organisms of the invention can contain the enzymes for performing one or more of these conversion steps and be able to carry out one or more of the conversions. The conversions can be performed by contacting the substrate with the indicated enzyme to produce the indicated product. The list uses the commonly known abbreviations for fatty acids.

palmitic acid 16:0 (PA) into produce stearic acid 18:0 (SA) using an elongase;

stearic acid 18:0 (SA) into oleic acid 18:1 (Δ9) (OA) using a Δ9-desaturase;

oleic acid 18:1 (Δ9) (OA) into linoleic acid 18:2 (Δ9,12) (LA) using a Δ12-desaturase;

linoleic acid 18:2 (Δ9,12) (LA) to 18:3 (Δ6,9,12) (GLA) using a Δ6-desaturase, converting linoleic acid into gamma linoleic acid;

18:3 (Δ6,9,12) (GLA) into 20:3 (Δ8,11,14) (DGLA) using a Δ6-elongase, converting gamma-linoleic acid into dihomo-γ-linoleic acid;

20:3 (Δ8,11,14) (DGLA) into 20:4 (Δ5,8,11,14) (ARA) using a Δ5-desaturase, converting dihomo-γ-linoleic acid into arachidonic acid;

20:4 (Δ5,8,11,14) (ARA) into a 22:4(Δ7,10,13,16) (DTA) using a Δ5-elongase, converting arachidonic acid into docosatetranoic acid (DTA or adrenic acid);

22:4(Δ3,10,13,16) (DTA) into a 22:5(Δ4,7,10,13,16) (DPAn6) using a Δ4-converting docosatetranoic acid into docosapentanoic acid;

18:2 (Δ9,12) (LA) into 18:3(Δ9,12,15) (ALA) using a ω3-desaturase, converting linoleic acid into alpha-linoleic acid;

18:3 (Δ6,9,12) (GLA) into 18:4(Δ6,9,12,15) (SDA) using an ω3-desaturase, converting gamma-linoleic acid into stearidonic acid;

20:3 (Δ8,11,14) (DGLA) into a 20:4(Δ8,11,14,17) (ETA) using an ω3-desaturase, converting dihomo-gamma-linoleic acid into eicosatetranoic acid;

20:4 (Δ5,8,11,14) (ARA) into a 20:5(Δ5,8,11,14,17) (EPA) using an ω3-desaturase, converting arachidonic acid into eicosapentanoic acid;

22:4(Δ7,10,13,16) (DTA) into 22:5 (Δ7,10,13,16,19) (DPA) using an ω3-desaturase, Δ19-desaturase, converting docosatetranoic acid into docosapentanoic acid;

22:5(Δ4,7,10,13,16) (DPAn6) into 22:6 (Δ4,7,10,13,16,19) (DHA) using an ω3-desaturase, Δ19-desaturase converting docosapentanoic acid into docosahexanoic acid;

18:3 (Δ9,12,15) (ALA) into 18:4 (Δ6,9,12,15) (SDA) using a Δ6-desaturase converting alpha-linoleic acid into stearidonic acid;

18:4 (Δ6,9,12,15) (SDA) into 20:4 (Δ8,11,14,17) (ETA) using a Δ6-elongase converting stearidonic acid into eicosatetranoic acid;

20:4 (Δ5,8,11,14) (ETA) into 20:5 (Δ5,8,11,14,17) (EPA) using a Δ5-desaturase converting eicosatetranoic acid into eicosapentanoic acid;

20:5 (Δ5,8,11,14,17) (EPA) into 22:5 (Δ7,10,13,16,19) (DPA) using a Δ5-elongase converting eicosapentanoic acid into docosapentanoic acid;

22:5 (Δ7,10,13,16,19) (DPA) into 22:6 (Δ4,7,10,13,16,19) (DHA) using a Δ4-desaturase converting docosapentanoic acid into docosahexanoic acid;

18:3 (Δ9,12,15) (ALA) into 20:3(Δ11,14,17) (ETE) using an Δ9-elongase converting alpha-linoleic acid into eicosatrienoic acid;

20:3(Δ11,14,17) (ETE) into 20:4 (Δ8,11,14,17) (ETA) using a Δ8-desaturase converting eicosatrienoic acid into eicosatetranoic acid; and

18:2 (Δ9,12) (LA) into 20:2 (Δ11,14) eicosadienoic acid (EDA) using a Δ9-elongase;

20:2 (Δ11,14) eicosadienoic acid into 20:3 (Δ8,11,14) (DGLA) using a Δ8-desaturase.

Each of the heterologous enzymes can perform a substrate to product conversion step, meaning that through the action of the enzyme a substrate is converted into a product, with or without the presence of cofactors. In some embodiments of the invention the product of one enzyme can be the substrate for another enzyme, and either or both of the enzymes can be heterologous to the cell where the reaction is occurring. In some embodiments the product of one heterologous enzyme is the substrate for another heterologous enzyme, and in other embodiments the products of at least two or at least three or at least four or at least five or at least six or at least seven heterologous enzymes are the substrates for at least two or at least three or at least four or at least five or at least six or at least seven other heterologous enzymes, any or all of which can be expressed in the cell or organism from an exogenous nucleic acid. In some embodiments the product of one enzyme is the substrate for the next consecutive enzyme in the pathway, as depicted in FIG. 1 and consecutive conversions can occur through at least two or three or four or five or six or seven enzymes in the pathway or network. In such manner a pathway, chain, or web of enzymatic reactions can be created in the cell. A pathway leads from a defined substrate to a defined product. A substrate or a product can be any described in FIG. 1 or otherwise herein. Such pathways, chains, or networks can also include one or two or three or more natural or native enzymes, i.e. enzymes naturally present in the cell or organism. Thus, exogenous enzymes can work with both other exogenous enzymes as well as with native enzymes to move a substrate forward along a pathway or network.

### Multiple Product Pathways

The cells or organisms of the invention can contain one or more of the pathways, chains, or networks of substrate to product conversion steps described herein, which can be utilized to produce any PUFA product. Any one or more (or all) of the steps can be performed by a heterologous enzyme, which can also be an exogenous enzyme. FIG. 1 depicts an example of a network of the invention composed of various pathways or reaction chains. In various embodiments any of the substrates can be selected as a starting point to produce any of a wide variety of products using the substrate to product conversion steps as disclosed herein. Thus, in some examples, LA or PA or SA or ALA can be identified as a substrate and utilized according to the invention to produce a product of, for example, ARA or EPA or DHA. The product can be produced by using one or more steps set forth in FIG. 1 to create a pathway from substrate to product. The person of ordinary skill with reference to this disclosure will understand that any substrate disclosed herein can be utilized in a pathway or network of the invention to produce any product disclosed herein.

A pathway converts a particular substrate into a particular product. Pathways can have one step or two steps or three steps or four steps or five steps or six steps or seven steps or more than seven steps, each step comprising a substrate to product enzymatic conversion. Pathways can trace a line from any substrate to any product, several examples of which are apparently from FIG. 1, and can use any combination of enzymes, e.g. any desaturases and any elongases as depicted in FIG. 1. In some non-limiting examples the pathways, chains, or networks of the invention involve conversion steps of LA to GLA using a Δ6-desaturase, GLA to DGLA using a M-elongase, DGLA to ARA using a Δ5-desaturase to produce ARA. A further step can be performed converting ARA to EPA. Two or more pathways comprise a network.

In another non-limiting example the pathway can be converting GLA into SDA using an ω3-desaturase, converting SDA into ETA using a Δ6-elongase, and converting ETA into EPA using a Δ5-desaturase.

In another example the pathway can be one or more of a) converting LA into GLA using a M-desaturase, converting GLA into DGLA using a Δ6-elongase, converting DGLA into ARA using a Δ5-desaturase, converting ARA into EPA using a ω3-desaturase; or b) converting LA into ALA using a 0-desaturase, converting ALA into SDA using a Δ6-desaturase, converting SDA into ETA using a Δ6-elongase, converting ETA into EPA using a Δ5-desaturase; or c) converting LA into GLA using a Δ6-desaturase, converting GLA into SDA using a ω3-desaturase, converting SDA into ETA using a Δ6-elongase, converting ETA into EPA using a Δ5-desaturase; or d) converting LA into GLA using a Δ6-desaturase, converting GLA into DGLA using a Δ6-elongase, converting DGLA into ETA using a Δ5-desaturase, converting ETA into EPA using an ω3-desaturase, to thereby convert LA into EPA or e) converting PA into SA using a C16-elongase, converting SA into OA using a Δ9-desaturase, converting OA into LA using a Δ12-desaturase. Any of the pathways can also be linked to another of the pathways.

Any of the pathways, chains, or networks disclosed herein can also comprise steps of a) converting EPA into DPA using a Δ5-elongase and/or b) converting DPA into DHA using a Δ4-desaturase. They can also comprise steps of a) converting ARA into DTA using a Δ5-elongase, and/or b) converting DTA into DPAn6 using a Δ4-desaturase.

### Cells/Organisms and Constructs

The recombinant cells or organisms of the invention can contain one or more pathways, chains, or networks as described herein. A recombinant cell is one that is expressing a recombinant nucleic acid, which can be an exogenous nucleic acid coding for one or more enzymes, which can be heterologous enzymes. In some embodiments the cell expresses at least two or at least three or at least four or at least five or at least six or at least seven heterologous enzymes, any one or more of which can be expressed from an exogenous nucleic acid. The enzymes can be coded and/or expressed from a construct, plasmid or other vector that has been transformed into the recombinant cell, or can be integrated into the genome of the cell. The recombinant cells or organisms of the invention can contain or express an exogenous nucleic acid construct or plasmid of the invention, or functionally can express one or more nucleic acid or polypeptide sequences of SEQ ID NOs: 1-52, or any nucleic acid or protein/peptide disclosed herein.

The examples provide various nucleic acid constructs or vectors that can be utilized in the present invention, and the constructs can contain a promoter operably linked to a nucleic acid sequence encoding a heterologous enzyme including, but not limited to, those heterologous enzymes disclosed herein. In one embodiment the nucleic acid sequence is one or more of SEQ ID NO: 27-52 and complements thereof or a nucleic acid sequence coding for a protein sequence of SEQ ID NO: 1-26 and complements thereof, but the nucleic acid can be any described herein. Any of the sequences described herein can also be present on a construct and can be operably linked to a promoter sequence and/or terminator sequence. In various embodiments the recombinant cells or organisms of the invention can perform at least one or at least two or at least three or at least four or at least five or at least six or at least seven substrate to product conversion steps described herein using one or more heterologous enzyme(s). One or more of the heterologous enzymes can be coded onto the construct or plasmid.

The recombinant cell or organism of the invention can be any suitable organism but in some embodiments is a Labyrinthulomycetes cell and the promoter (and terminator) can be any suitable promoter and/or terminator and in any combination, for example any promoter described herein or other promoters that may be isolated from Labyrinthulomycetes or derived from such sequences, in combination with any terminator described here or other terminators determined to permit gene expression in the recombinant cell or organism. For example, terminator sequences may be derived from organisms including, but not limited to, heterokonts (including Labyrinthulomycetes, fungi, and other eukaryotic organisms. In various embodiments the promoter and/or terminator is any one operable in a cell or organism that is a Labyrinthulomycetes, including any genus thereof. Any of the constructs can also contain one or more selection markers, as appropriate. In one embodiment the recombinant cells or organisms of the invention do not require the presence a fatty acid or a PUFA in the growth medium to grow and remain viable. In other embodiments the recombinant cells or organisms of the invention do not require the presence of other lipid molecules in the growth medium, such as glycerolipids, glycerophospholipids, or any PUFA bearing lipid molecule in order to grow and remain viable.

In a specific embodiment a construct or vector of the invention has one or more of an Hsp60-788 promoter, and/or a Tsp-749 promoter and/or a Tubα-738 promoter and/or a Tubα-997 promoter. The construct or vector can also have one or more of an ENO2 terminator and/or a PGK1 terminator. Any combination of promoters and/or terminators can be used but in one embodiment the construct or vector has a Tubα-997 promoter and a PGK1 terminator. This construct or vector can be utilized to express any desaturase or elongase, including but not limited to, a Δ4 or a Δ5 or a Δ6 or a Δ8 or a Δ9 or an ω3 desaturase, or a Δ5 or Δ6 or Δ9-elongase. The promoters and/or terminators can be operably linked to any one or more nucleic acid sequences described herein, for example those encoding a heterologous enzyme. In one embodiment the sequences can be any one or more of the nucleic acids described herein. The sequence of the Tubα-997 promoter is provided as SEQ ID NO: 53 and the sequence of the PGK1 terminator as SEQ ID NO: 54.

In addition to the promoters and/or terminators described herein the promoter and/or terminator can also be one having at least 70% or at least 80% or at least 90% or at least 95% or at least 97% or at least 98% or at least 99% or 80-99% or 90-99% or 90-95% or 95-97% or 95-98% or 95-99% sequence identity to a sequence of SEQ ID NO: 53-54 or to complements thereof. Any of the promoter and/or terminator sequences can also have less than 100% sequence identity with a nucleic acid sequence of SEQ ID NO: 53-54 or complements thereof.

Any of the promoter and/or terminator sequences can also have at least one substitution modification relative to a nucleic acid sequence of SEQ ID NO: 53-54 or a complement thereof, but can also have at least 2 or at least 3 or at least 4 or at least 5 or at least 6 or at least 7 or at least 8 or at least 9 or at least 10 or 1-5 or 5-10 or 10-50 or 25-50 or 30-100 or 50-100 or 50-150 or 100-150 substitution modifications relative to a nucleic acid sequence of SEQ ID NOs: 53-54. Any of the promoter and/or terminator sequences of the invention can be operably linked to any nucleic acid described herein.

In additional embodiments a construct of the invention contains a C16 elongase or a M-desaturase or a Δ8-desaturase under the control of a Tubα-997 promoter and a SV40 terminator of Simian virus SV40 (SV40t). The construct can also have a Δ9-desaturase or a Δ6-elongase or a Δ9-elongase under the control of a RPL11-699p promoter and an ENO2t terminator. The construct can also have a Δ12-desaturase or a Δ5-desaturase under the control of a Hsp60-788p promoter and a PGK1t terminator.

The invention also provides a recombinant cell or organism that contains a nucleic acid construct or plasmid of the invention or expresses one or more of the constructs or nucleic acids or proteins or peptides of the invention, as described herein. In some embodiments the recombinant cell or organism expresses 2 or 3 or 4 nucleic acids or polypeptides described herein. The recombinant cells or organism can also contain and functionally express two or three or more constructs of the invention.

In various embodiments the cells or organisms described herein produce a FAME profile having the percent of a specific PUFA (on a “by weight” basis). In some embodiments the cells or organisms of the invention are highly oleaginous and have greater than 40% lipid or greater than 50% lipid or greater than 60% lipid or greater than 70% lipid by weight of dry cell weight (DCW).

### Strain Engineering

According to the invention various strains of organisms of the class Labyrinthulomyces can be created according to the invention to provide for a specific need. In one embodiment the invention provides an organism of the class Labyrinthulomycetes that has a PKS system that produces DHA disrupted, deleted, or impaired so that the organism produces a reduced amount of DHA or does not produce DHA versus the unmodified cell or organism. The cell or organism can contain the FAS system producing C16:0 and an elo/des pathway engineered into the organism according to the invention so that the organism produces the enhanced amounts of ARA or EPA as described herein.

In another embodiment the native (wild type) organism can have a native PKS pathway producing DHA, which can be engineered according to the invention to be disrupted, deleted, or impaired. The organism can also be engineered to have a PKS system producing EPA according to the invention resulting in a strain producing EPA.

In another embodiment the organism can have no native PKS system that produces DHA. The native cell can have a pathway that converts ARA or EPA into DHA as some Labyrinthulomycetes cells do. But when desirable to produce ARA or EPA and to produce less or no DHA, the organism can be engineered according to the invention so that the portion of the pathway converting ARA or EPA into DHA is disrupted, deleted, or impaired. Thus the organism produces ARA or EPA and produces a lesser amount or no DHA, compared to the non-engineered organism.

In another embodiment the organism can have a native PKS pathway producing DHA that is disrupted, deleted, or impaired and a PKS system producing EPA can be engineered into the organism according to the invention resulting in a strain producing EPA. The organism can also have a native elo/des pathway producing ARA or EPA. It can further have a pathway converting ARA or EPA to DHA. In this organism both the elo/des pathway and the pathway converting ARA or EPA to DHA (if present) can be disrupted, deleted, or impaired according to the invention, to result in an organism that produces EPA and produces less or no DHA.

### PUFA and FAME Profiles

The analysis of fatty acid content in biological materials is a common task in lipid research and its methods are understood by persons of ordinary skill in the art. In various embodiments the recombinant cells and organisms of the invention produce unique or advantageous fatty acid or PUFA profiles. A fatty acid or PUFA profile is a distribution of fatty acids or PUFAs produced by the organism. One manner of describing a fatty acid or PUFA profile produced by an organism or cell is in terms of the fatty acid methyl ester percent (FAME) profile, sometimes referred to as “microbial fingerprinting” since different organisms or cells can produce different fatty acids and in different combinations, resulting in distinct FAME profiles that can be used to distinguish and characterize the fatty acids produced by different cells or organisms.

Fatty acid methyl esters (FAME) are a type of fatty acid ester derived by transesterification of fats with methanol. The FAME profile is an accepted and reliable manner to indicate the quantity of a fatty acid (or PUFA) produced by a cell. FAME profiles are expressed by weight. Thus, when a composition contains, for example, a FAME profile of more than 12% OA, it indicates that more that 12% of the FAME is OA by weight. The FAME profile can be determined by any method generally accepted by persons of ordinary skill in the art. FAME profiles can be determined for whole cells, biomass, or microbial oils. In addition to the FAME profile any other method of calculating the percent of the total fatty acids or total cellular lipids produced by a cell or organism can also be used, and the percentages of particular fatty acids achieved can also be applied with such other methods.

The recombinant cells or organisms of the invention produce advantageous amounts of desirable fatty acids or PUFAs, which is reflected in the FAME profiles. DHA is a valuable nutritional oil, but in some applications it is desirable to produce an oil with a lower amount of DHA or with no DHA. In some embodiments the cells or organisms of the invention produce microbial oils and produce little or no DHA in the microbial oil, or produce a reduced amount of DHA relative to the wild type or non-engineered cell or organism. In one embodiment the cells or organisms of the invention do not produce DHA as the most prevalent PUFA, or the primary PUFA produced is a PUFA other than DHA. In some embodiments the cells or organisms produce a FAME profile having less than 25% or less than 15% or less than 10% or less than 5% or less than 1% of DHA or no DHA. In various embodiments the recombinant cells or organisms produce amounts of OA or PA or ARA or EPA described herein and produce a FAME profile having less than 15% or less than 12% or less than 10% or less than 5% or less than 2% DHA.

Alternatively in some embodiments the cells or organisms have a composition such that the total fatty acids of the cells or organisms is less than 25% or less than 15% or less than 10% or less than 5% or less than 1% DHA, or the total fatty acids of the cell or organism do not comprise DHA. In various embodiments the recombinant cells or organisms produce amounts of OA or PA or ARA or EPA described herein and a total fatty acids content of less than 15% or less than 12% or less than 10% or less than 5% or less than 2% or less than 1% DHA. Alternatively in some embodiments the cells or organisms have a PUFA composition such that less than 25% or less than 15% or less than 10% or less than 5% or less than 1% of the total lipids in the cell are DHA or the total lipids in the cell do not comprise DHA. In various embodiments the recombinant cells or organisms produce amounts of OA or PA or ARA or EPA described herein and the total lipids in the cell comprise less than 15% or less than 12% or less than 10% or less than 5% or less than 2% or less than 1% DHA.

Labyrinthulomycetes that cannot make their own DHA require the supplementation of a lipid-containing molecule, fatty acid, PUFA, or DHA in the medium in order to grow and remain viable. A supplement is a component added to the growth medium of an organism. In various embodiments the cells or organisms of the invention do not require the presence of a fatty acid in the medium to grow and remain viable. A cell is viable when it is capable of sustained reproduction and multiplication of the numbers of the cells. In some embodiments the cells or organisms of the invention do not require the presence of a PUFA in the growth medium, or do not require the presence of DHA in the growth medium.

In some specific embodiments the recombinant cells or organisms of the invention can have a variety of desirable PUFA profiles such as, for example, a FAME profile having greater than 8% or greater than 10% or greater than 12% or greater than 15% or greater than 18% or greater than 25% EPA. In another embodiment the cells or organisms have a FAME profile of greater than 12% ARA or greater than 15% ARA or greater than 18% ARA or greater than 20% ARA or greater than 25% ARA or greater than 30% ARA or 10-20% ARA or 10-25% ARA or 10-30% ARA or 10-40% ARA. In another embodiment the cells or organisms have a FAME profile that is greater than 12% OA or greater than 15% OA or greater than 18% OA or greater than 20% OA or greater than 25% OA or greater than 30% OA or 10-20% OA or 10-25% OA or 10-30% OA or 10-40% OA. In another embodiment the cells or organisms have a FAME profile that is greater than 15% PA or greater than 18% PA or greater than 20% PA or greater than 25% PA or greater than 30% PA. In another embodiment the cells or organisms have a FAME profile that is greater than 15% SA or greater than 18% SA or greater than 20% SA or greater than 25% SA or greater than 30% SA or greater than 35% SA or greater than 405 SA. Any of the above cells or organisms can also have a FAME profile that is less than 25% or less than 20% or less than 12% or less than 10% or less than 5% or less than 2% or less than 1% DHA or that has no DHA.

The fatty acid profile of a cell or organism shows the distribution of the total fatty acids in a cell or organism. In some specific embodiments the recombinant cells or organisms of the invention can have a total fatty acid profile having greater than 8% or greater than 10% or greater than 12% or greater than 15% or greater than 18% or greater than 25% EPA. In another embodiment the cells or organisms have a total fatty acid profile of greater than 12% ARA or greater than 15% ARA or greater than 18% ARA or greater than 20% ARA or greater than 25% ARA or greater than 30% ARA. In another embodiment the cells or organisms have a total fatty acid profile that is greater than 12% OA or greater than 15% OA or greater than 18% OA or greater than 20% OA or greater than 25% OA or greater than 30% OA. In another embodiment the cells or organisms have a total fatty acid profile that is greater than 15% PA or greater than 18% PA or greater than 20% PA or greater than 25% PA or greater than 30% PA. In another embodiment the cells or organisms have a total fatty acid profile that is greater than 15% SA or greater than 18% SA or greater than 20% SA or greater than 25% SA or greater than 30% SA or greater than 35% SA or greater than 40% SA. Any of the above cells or organisms can also have a total fatty acid profile that is less than 25% or less than 20% or less than 12% or less than 10% or less than 5% or less than 2% or less than 1% DI-IA or that has no DHA. Methods of determining the total fatty acid profile of a cell are known by persons or ordinary skill in the art.

In some specific embodiments the recombinant cells or organisms of the invention can have total cellular lipids greater than 8% or greater than 10% or greater than 12% or greater than 15% or greater than 18% or greater than 25% EPA. In another embodiment the cells or organisms have total cell lipids of greater than 12% ARA or greater than 15% ARA or greater than 18% ARA or greater than 20% ARA or greater than 25% ARA or greater than 30% ARA. In another embodiment the cells or organisms have total cellular lipids greater than 12% OA or greater than 15% OA or greater than 18% OA or greater than 20% OA or greater than 25% OA or greater than 30% OA. In another embodiment the cells or organisms have total cellular lipids greater than 15% PA or greater than 18% PA or greater than 20% PA or greater than 25% PA or greater than 30% PA. In another embodiment the cells or organisms have total cellular lipids greater than 15% SA or greater than 18% SA or greater than 20% SA or greater than 25% SA or greater than 30% SA or greater than 35% SA or greater than 40% SA. Any of the above cells or organisms can also have total cellular lipids less than 25% or less than 20% or less than 12% or less than 10% or less than 5% or less than 2% or less than 1% DHA or having no DHA. Methods of determining total cellular lipids are known by persons or ordinary skill in the art.

FAME profiles are a preferred method of determining fatty acids or PUFAs in a cell. FAME profiles can be determined by the following method. At the end of the culture period, cells were harvested and aliquots were analyzed for FAME. For biomass assessment, 4 ml of fermentation broth was pipetted to a pre-weighed 15 ml conical centrifuge tube. The tube containing the culture aliquot was centrifuged at 3220×g for 20 min, and the supernatant was decanted. The pellet was then frozen at −80° C. overnight, followed by freeze drying for 16-24 h. The conical centrifuge tube with dried pellet inside was weighed, and the weight of the lyophilized pellet, was calculated by subtracting the weight of the empty tube. The lyophilized pellet weight was standardized by dividing by the aliquot volume (4 ml) to obtain a value for the biomass per ml of culture.

Fatty acid methyl esters (FAME) were assessed using gas chromatography to analyze the fatty acid content of triplicate 50 to 200 μL volume aliquots of the cultures. The culture aliquots were diluted 1:10 in 1×PBS prior to aliquoting and drying for FAME sample preparation. The samples were dried via a centrifugal evaporator (HT-4X GENEVAC®) and stored at −20° C. until prepped for fatty acid methyl ester analysis. For extraction, 0.5 mL of 5M potassium hydroxide in methanol and 0.2 mL tetrahydrofuran containing 25 ppm butylated hydroxy toluene were added to the samples. Next, 80 uL of a 2 mg/mL C11:0 free fatty acid, C13:0 triglyceride, and C23:0 fatty acid methyl ester internal standard mix in n-heptane was added. After about 0.5 mL of 425-600 μm acid washed glass beads were added, the samples were placed into a tissue homogenizer (GENO/GRINDER®) at 1200 rpm for 10 min. The samples were then heated at 80° C. for 30 min and this was followed by 5 min at 1200 rpm in the homogenizer. Methanol containing 14% boron trifluoride was then added to the samples and they were returned to the 80° C. heating block for 30 min. The samples were then put into the homogenizer again at 1200 rpm for 5 min, vortexed once again at 2500 rpm for 5 min. Lastly, 2 mL of n-heptane and 0.5 mL 5M (saturated) sodium chloride were added and the samples were put into a homogenizer for 1.5 min at 1200 rpm and vortexed a final time at 2500 rpm for 5 min. The racks were then centrifuged at 1000 rpm for 1 min after which the top layer was sampled by a GERSTEL® MPS auto-sampler paired to a 7890 AGILENT® GC unit equipped with a flame ionization detector. A 10 m×0.1 mm×0.1 um DB-FFAP column (a nitroterephthalic-acid-modified polyethylene glycol column of high polarity) from AGILENT® was used. While the FAME analysis can be performed by the above described method, any generally accepted method of measuring a FAME profile can also be used such as, for example, AOCS methods Ce 1b-89 (Fatty Acid Composition of Marine Oils by GLS) or Ce 1-62 (Fatty Acid Composition by Packed Column Gas Chromatography). Those of ordinary skill in the art will understand other methods that can be used.

### Microbial Oil

The recombinant cells or organisms of the invention allow for the production of microbial oil having high amounts of desirable PUFAs and/or low amounts of less desirable PUFAs, depending on the desired amounts of specific PUFAs in specific applications. The amounts of specific PUFAs produced by the recombinant cells or organisms of the invention can be adjusted to desired levels or ratios. In various embodiments the recombinant cells or organisms of the invention produce a microbial oil containing OA, or PA or ARA or SA or EPA. The microbial oils of the invention can produce a FAME profile or total fatty acid profile or have total cellular lipids having greater than 5% or greater than 10% or greater than 20% or greater than 30% or greater than 40% or greater than 50% or from 5-10% or from 5-11% or from 5-15% or from 5-20% or from 10-15% or from 10-20% or from 10-30% or from 10-60% or from 12-18% or from 15-20% or from 18-25% or from 20-25% or from 20-30% or from 25-40% or from 30-40% or from 30-50% of any of OA or PA or ARA or SA or EPA. Any of the microbial oils can also have a FAME profile or total fatty acid profile or total cellular lipids of less than 15% DHA or less than 10% DHA or less than 5% DHA or less than 2% DHA or less than 1% DHA or no DHA. In some embodiments the recombinant cells or organisms of the invention produce no DHA or produce a FAME profile or total fatty acid profile showing no DHA. Any of the microbial oils described herein can be derived from the cells or organisms of the invention described herein. In a particular embodiment the microbial oil derived from the cells or organisms of the invention have a FAME profile or total fatty acids profile or total cellular lipids having greater than 10% EPA and less than 1% DHA.

The microbial oil produced by or derived from the recombinant cells or organisms of the invention can be a microbial oil produced by or derived from only the recombinant cells or organisms of the invention. The oils can contain OA or PA or ARA or EPA, or other PUFAs. The microbial oil can have a FAME profile or a total fatty acids profile or total cellular lipids of with a higher amount of EPA than DHA. In some embodiments the cells or organisms or biomass or microbial oils of the invention produce a FAME profile or a total fatty acid profile or total cellular lipids having at least 5% EPA or at least 8% EPA or at least 10% EPA at least 12% EPA or at least 15% EPA or at least 20% EPA or at least 25% EPA or from 0-15% EPA or from 5-15% EPA or from 5-11% EPA or from 8-15% EPA or from 5-20% EPA or from 5-25% EPA or from 10-15% EPA. The cells or organisms or biomass or microbial oils can also have a FAME profile or total fatty acid profile or total cellular lipids having less than 15% DHA or less than 10% DHA or less than 5% DHA or less than 2% DHA or less than 1% DHA or no DHA. Any of the microbial oils described herein can also be combined with one or more other oils or substances derived from other sources to provide an oil mixture.

In various embodiments the microbial oils or biomass of the invention can be an unconcentrated oil or biomass, meaning that it is derived or extracted from the recombinant cells or organisms of the invention in the stated form and without further steps to concentrate or purify the oil or biomass. In one embodiment the microbial oils or biomass of the invention do not contain a contaminating heavy metal such as, for example, chromium, cobalt, nickel, copper, zinc, arsenic, selenium, silver, cadmium, antimony, mercury, thallium, or lead.

### Biomass

The present invention also provides a biomass made with or derived from the recombinant cells or organisms of the invention. Biomass is biological material derived from the cells or organisms of the invention. The biomass can be wet biomass or dry biomass, and in some embodiments the biomass of the invention is reduced to a pellet with excess liquids removed. It can also optionally be dried to remove some or all residual liquid to form a dry biomass. The biomass can be obtained by growing the recombinant cells or organisms of the invention to a desired amount. The recombinant cells or organisms can be obtained from conventional cell culture or fermentation or any means of culturing or amplifying the cells or organisms of the invention. Because the recombinant cells or organisms of the invention produce desirable or advantageous amounts of PUFAs and/or have an advantageous FAME profile or total fatty acid profile or total lipids profile, the biomass made from the cells or organisms will also have advantageous amounts of PUFAs. The amounts are advantageous in some embodiments because of the large amount of specific PUFAs they contain. In other embodiments they are advantageous because of the low amounts of less desirable PUFAs they contain. They can also be advantageous because of the relative amounts of different PUFAs they contain. The biomass of the invention can have any of the same PUFA amounts, ratios, FAME profiles, total fatty acid profiles, or total cellular lipids profiles described herein with respect to the recombinant cells or organisms or microbial oils of the invention.

### Food Products

The cells or organisms or biomass or microbial oils of the invention can also be utilized in various food products either as a complete food or as a food ingredient. The food products can be any food product, examples including animal feed, aquaculture feed, a nutritional oil, infant formula, or a human food product that contains a microbial oil or biomass of the present invention. Additionally, other nutritive components can be contained in the food product and the biomass or microbial oils of the invention can be one ingredient or an additive in a food product. The food products or ingredients of the invention can also include preservatives, fillers, or other acceptable food ingredients. The food products or ingredients of the invention can contain biomass of the invention combined with other foods such as, for example, grains or proteinaceous food products or ingredients or one or more sugars, or food colorings or flavorings. The food products or ingredients of the invention can also be provided in an acceptable food wrapping, bag, or container.

Since various fatty acids are an essential component of the human diet the microbial oils of the invention can also be utilized as a dietary supplement or as an ingredient in a dietary supplement. Additional uses of the microbial oils of the invention include use as or in a pharmaceutical product or pharmaceutical intermediate. Pharmaceuticals containing a microbial oil of the invention can be for oral or intravenous administration. In some exemplary embodiments the microbial oils of the invention are useful in pharmaceutical products for the treatment of high blood pressure, blood thinners, macular degeneration, heart disease or irregular heartbeats, schizophrenia, personality disorders, cystic fibrosis, Alzheimer's disease, depression, or diabetes.

### Nucleic Acid and Peptide Sequences

The present invention also provides polypeptide sequences of various enzymes useful in the invention and nucleic acid sequences coding for them, and functional fragments of any of them. Table 14 lists SEQ ID NOs: 1-26, the type of polypeptide, and its source. The invention also provides isolated, recombinant nucleic acids of SEQ ID NOs: 27-52 and complements thereof, and nucleic acid sequences or functional RNA sequences that code for a polypeptide of SEQ ID NOs: 1-26 and complements thereof. The invention also provides isolated recombinant nucleic acid sequences having at least 70% or at least 80% or at least 90% or at least 95% or at least 97% or at least 98% or at least 99% or 80-99% or 90-99% or 90-95% or 95-97% or 95-98% or 95-99% sequence identity to a sequence of SEQ ID NO: 27-52 or to complements thereof, or said sequence identities to a nucleic acid sequence or functional RNA sequence coding for a polypeptide sequence of SEQ ID NO: 1-26 and complements of such nucleic acid sequences. Any of the nucleic acid sequences can also have less than 100% sequence identity with a nucleic acid sequence of SEQ ID NO: 27-52 or complements thereof or to a nucleic acid sequence or functional RNA sequence coding for a polypeptide of SEQ ID NO: 1-26 or complements thereof.

Also disclosed are functional fragments of any of the nucleic acid or amino acid sequences recited herein. A functional fragment is one that performs at least 50% of the action as the disclosed full sequence. For example, a functional fragment of a nucleic acid sequence that encodes a functional protein with X activity would encode a fragment of that protein having at least 50% of X activity. A functional fragment of an amino acid sequence would have at least 50% of the activity of the disclosed full sequence. When the activity is a binding activity, the functional fragment would bind the same epitope with at least 50% of the binding activity as the disclosed full sequence. When the amino acid sequence activity is a signal activity, the fragment would provide at least 50% of the signal activity of the disclosed full sequence. In various embodiments functional fragments can have at least 50% or at least 60% or at least 70% or at least 80% of at least 90% of the length of the disclosed full sequence.

The invention also provides polypeptides of SEQ ID NO: 1-26 and polypeptides having at least 70% or at least 80% or at least 90% or at least 95% or at least 97% or at least 98% or at least 99% or 80-99% or 90-99% or 95-97% or 95-98% or 95-99% sequence identity to a sequence of SEQ ID NO: 1-26. Any of the polypeptide sequences can have less than 100% sequence identity to a sequence of SEQ ID NO: 1-26.

Any of the sequences can also have at least one substitution modification relative to a nucleic acid sequence of SEQ ID NO: 27-52 or a complement thereof, or to a polypeptide sequence of SEQ ID NO: 1-26, but can also have at least 2 or at least 3 or at least 4 or at least 5 or at least 6 or at least 7 or at least 8 or at least 9 or at least 10 or 1-5 or 5-10 or 10-50 or 25-50 or 30-100 or 50-100 or 50-150 or 100-150 substitution modifications relative to a nucleic acid sequence of SEQ ID NOs: 27-52 or to a complement thereof or to a polypeptide sequence of SEQ ID NO: 1-26. Any of the nucleic acid sequences of the invention can be functionally expressed by a recombinant cell or organism of the invention, and can be operably linked to a suitable promoter and/or terminator sequence. Any of the polypeptide sequences disclosed herein can be functionally expressed in a recombinant cell or organism of the invention.

The invention also provides isolated, recombinant nucleic acid sequences having the percent sequence identities recited herein and above to a nucleic acid sequence having at least 50 contiguous nucleotides or at least 100 or at least 200 or at least 300 or at least 500 or at least 700 or at least 100 contiguous nucleotides to a nucleic acid sequence of SEQ ID NO: 27-52 or to a complement thereof, or to a nucleic acid sequence or functional RNA sequence that codes for a polypeptide of SEQ ID NO: 1-26 or a complement of such sequences.

The terms, “sequence identity” or percent “identity” in the context of two or more nucleic acids or polypeptide sequences, refer to two or more sequences or subsequences that are the same or have a specified percentage of amino acid residues or nucleotides that are the same, when compared and aligned for maximum correspondence over a comparison window. Unless otherwise specified, the comparison window for a selected sequence, e.g., “SEQ ID NO: X” is the entire length of SEQ ID NO: X, and, e.g., the comparison window for “100 bp of SEQ ID NO: X” is the stated 100 bp. The degree of amino acid or nucleic acid sequence identity can be determined by various computer programs for aligning the sequences to be compared based on designated program parameters. For example, sequences can be aligned and compared using the local homology algorithm of Smith & Waterman Adv. Appl. Math. 2:482-89, 1981, the homology alignment algorithm of Needleman & Wunsch J. Mol. Biol. 48:443-53, 1970, or the search for similarity method of Pearson & Lipman Proc. Nat'l. Acad. Sci. USA 85:2444-48, 1988, and can be aligned and compared based on visual inspection or can use computer programs for the analysis (for example, GAP, BESTFIT, FASTA, and TFASTA in the Wisconsin Genetics Software Package, Genetics Computer Group, 575 Science Dr., Madison, Wis.).

The BLAST algorithm, described in Altschul et al., J. Mol. Biol. 215:403-10, 1990, is publicly available through software provided by the National Center for Biotechnology Information. This algorithm identifies high scoring sequence pairs (HSPS) by identifying short words of length W in the query sequence, which either match or satisfy some positive-valued threshold score T when aligned with a word of the same length in a database sequence. T is referred to as the neighborhood word score threshold (Altschul et al., 1990, supra). Initial neighborhood word hits act as seeds for initiating searches to find longer HSPs containing them. The word hits are then extended in both directions along each sequence for as far as the cumulative alignment score can be increased. Cumulative scores are calculated for nucleotides sequences using the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below due to the accumulation of one or more negative-scoring residue alignments; or the end of either sequence is reached. For determining the percent identity of an amino acid sequence or nucleic acid sequence, the default parameters of the BLAST programs can be used. For analysis of amino acid sequences, the BLASTP defaults are: word length (W), 3; expectation (E), 10; and the BLOSUM62 scoring matrix. For analysis of nucleic acid sequences, the BLASTN program defaults are word length (W), 11; expectation (E), 10; M=5; N=−4; and a comparison of both strands. The TBLASTN program (using a protein sequence to query nucleotide sequence databases) uses as defaults a word length (W) of 3, an expectation (E) of 10, and a BLOSUM 62 scoring matrix. See, Henikoff & Henikoff, Proc. Nat'l. Acad. Sci. USA 89: 10915-19, 1989.

In addition to calculating percent sequence identity, the BLAST algorithm also performs a statistical analysis of the similarity between two sequences (see, e.g., Karlin & Altschul, Proc. Nat'l. Acad. Sci. USA 90:5873-87, 1993). The smallest sum probability (P(N)), provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, preferably less than about 0.01, and more preferably less than about 0.001.

### Example 1

### Isolation of Wild-Type Labyrinthulomycete Strains

A collection project that isolated hundreds of microorganisms for assessing lipid production was initiated. Wild-type strain isolation biotopes for sampling were identified based upon access via legal permits and the known biology of the class of organism. Biotopes were categorized as open ocean, estuary, coastal lagoon, mangrove lagoon, tide pool, hypersaline, freshwater, or aquaculture farm. Sampling location latitudes spanned the range from temperate, subtropical to tropical. Water samples collected included direct samples of 2 liters. In some cases, plankton tows were performed using a 10 μM net. A total of 466 environmental samples were collected from 2010-2012. Temperature ranged from 4° C. to 61° C., and pH ranged from 2.45 to 9.18. Dissolved oxygen ranged from 0 to 204% air saturation and salinity ranged from 0 ppt to 105 ppt. All samples were inoculated on site into 125 f/2 media (composition: 75 mg/L NaNO₃, 5 mg/L NaH₂PO₄.H₂O, 0.005 mg/L biotin, 0.01 mg/L CoCl₂.6H₂O, 0.01 mg/L CuSO4.5H₂O, 4 mg/L Na₂EDTA, 3 mg/L FeCl₂, 0.18 g/L MnCl₂, 0.006 mg/L Na₂MoO₄.2H₂O, 0.1 g/L thiamine, 0.005 mg/L vitamin B12, 0.022 mg/L ZnSO₄) and seawater-glucose-yeast-peptone (SWGYP) media (composition: 2 g glucose, 1 g peptone and 1 g Difco yeast extract per liter of sterile-filtered seawater) to initiate growth. A separate set of samples was generated in duplicate 50 ml aliquots that included 10% glycerol and subsequently frozen with dry ice. Finally, additional samples were frozen in 10% glycerol in a volume of 2 L for subsequent DNA isolations. The samples were shipped to the laboratory on the day of collection and arrived the following day for inoculation into fermentation broth in stationary or shake flasks. Carbenicillin, streptomycin, and nystatin were included in the cultures to retard the growth of bacteria or fungi.

Inoculated enrichments were incubated at a range of temperatures from 15° C. to 30° C. and subsequently plated onto SWGYP or f/2 agar with antibiotics. Isolated colonies were recovered, amplified in SWGYP or f/2 media and PCR amplification of 18s rDNA was performed to determine the taxonomic identity of the isolated microorganism.

### Example 2

Publicly available sequences of genes from the elongase/desaturase pathway were identified and used to identify homologs from various published sources. Some of the homologs were synthesized and cloned behind the GAL1 promoter of S. cerevisiae/E. coli shuttle vector pYES260 for characterization in yeast following galactose induction. Selected elongase/desaturase sequences were used to identify homologs.

TABLE 1
Example sequences of elongase/desaturase
sequences used to identify homologs

| Enzyme | Source | Accession # |
|---|---|---|
| Δ9-desaturase | Mortierella alpina | ADE06659 |
| | Phaeodactylum tricornutum | AAW70158 |
| | Plasmodium falciparum | XP_001351669 |
| | Trypanosoma cruzi | AEQ77281 |
| | A. thaliana | AAM63359 |
| | Y lipolytica | CAG81797 |
| Δ12-desaturase | T. aureum ATCC 34304 | BAM37464 |
| Δ6-desaturase | T. aureum | WO02081668 |
| Δ6-elongase | Thraustochytrium sp. | AX951565 |
| | Thraustochytrium sp. | AX214454/ |
| | | US7544859 |
| | Thraustochytrium sp. | US7544859 |
| | Thraustochytrium sp. | US7544859 |
| | T. aureum | |
| Δ5-desaturase | Thraustochytrium sp. | AF489588 |
| | ATCC21685 | |
| | T. aureum ATCC 34304 | US7241619 |
| | T. aureum BICC7091 | WO02081668 |
| ω3 desaturase | Saprolegnia diclina | AY373823 |
| | Caenorhabditis elegans | CAC44309 |
| | Saccharomyces kluyveri | AB118663 |
| | Mortierella alpina | AB182163 |
| | Phytophthora infestans | CAJ30870 |
| Δ12/Δ15-desaturase | Fusarium monoliforme | DQ272516 |
| Δ4-desaturase | Thraustochytrium sp. | AF489589 |
| | T. aureum | AF391543-5 |
| | T. aureum | AAN75707 |
| | T. aureum | AAN75708 |
| | T. aureum | AAN75709 |
| | T. aureum | AAN75710 |
| | Thraustochytrium sp. | AAM09688 |
| | ATCC21685 | |
| | T. aureum | US7045683 |
| | Schizochytrium aggregatum | US7045683 |
| Δ9-elongase | Thraustochytrium sp. | BAM66615 |
| | ATCC26185 | |

### Example 3

### Fatty Acid Feeding in S. cerevisiae

S. cerevisiae can import fatty acids and convert them to acyl-CoAs. S. cerevisiae does not elongate or desaturate PUFAs and can be used as a host for elongase/desaturase activity assays. S. cerevisiae cultures expressing candidate genes were inoculated into SD minus uracil medium supplemented with 20 g/L glucose and incubated at 30° C., 250 rpm for 24 hours. These cultures were then used to inoculate SD minus uracil medium supplemented with 20 g/L galactose, 1% tergitol solution (type NP-40, 70% in H₂O), and 0.5 mM of the test PUFA substrate. Cultures were normalized to a starting OD₆₀₀=0.2 and incubated for 24 hours at 30° C., 250 rpm. Prior to sampling for GC-FAME analysis, culture pellets were washed to remove residual medium. The activity of each enzyme on a given substrate was measured as the percent of the substrate converted to the product: % conversion=100×product (4/[product (μg)+substrate (μg)].

### Example 4

### Aurantiochytrium Expression Constructs

Genes encoding elongases and desaturases of interest were subcloned for expression and characterization in Aurantiochytrium. Labyrinthulomycetes promoters used for the expression of genes in the constructs are described herein. For characterization of individual elongase or desaturase gene candidates, the CDS coding for each enzyme was cloned between the Aurantiochytrium full-length tubulin alpha chain promoter (Tubα-997p) and the S. cerevisiae PGK1 terminator (PGK1t), and the expression cassette was linked to a nourseothricin-resistance cassette. Constructs containing more than one elongase and/or desaturase are described further below and summarized in Table 2. The promoters used in these constructs originated from regions immediately upstream of the genes tubulin alpha (Tubα-738p), mitochondrial chaperonin 60 (Hsp60-788p), 60s ribosomal protein (RPL11-699p), tetraspanin (Tsp-749p), and actin depolymerase (Adp-830p) of the Aurantiochytrium host strain. Genes sourced from non-Labyrinthulmycetes organisms were codon-optimized, using DNA synthesis, for expression in Aurantiochytrium. After sequence verification, each plasmid was linearized by restriction digest and electroporated into an Aurantiochytrium PUFA-auxotroph strain (Ex. 20) by inactivation of both alleles of either pfaA or pfaB. This strain does not produce DHA or other PUFAs. In this background, only trace amounts of omega-3 and delta-4 desaturase activities are detectable. No other elongase or desaturase activity has been observed in this strain.

TABLE 2
Genotypes of Constructs 1-7
Construct Genotype: promoter: CDS terminator

| | |
|---|---|
| 1 | Hsp60-788p: Seq. 2: PGK1tTsp-749p: Seq. 6: ENO2t Tubα- |
| | 738p: Seq. 9: PDC1t |
| 2 | Hsp60-788p: Seq. 13: PGK1tTsp-749p: Seq. 14: ENO2t Tubα- |
| | 738p: Seq. 17: PDC1t Adp-830p: Seq. 1: TDH35 |
| 3 | Tubα-997p: Seq. 17: PGK1t RPL11-699p: Seq. 14: ENO2t |
| 4 | Tubα-997p: Seq. 17: PGK1t |
| 5 | Tubα-997p: Seq. 17: PGK1t Tsp-749p: Seq. 14: ENO2t |
| 6 | Tubα-997p: Seq. 17: SV40t RPL11-699p: Seq. 14: ENO2t and |
| | Hsp60-788p: Seq. 13: PGK1t |
| 7 | Tubα-997p: Seq. 9: SV40t RPL11-699p: Seq. 6: ENO2t Hsp60- |
| | 788p: Seq. 2: PGK1t |

Electroporation method:

| | |
|---|---|
| Media: | FM001: FM002 solidified with 15 g/L bacto-agar. |
| | FM002: 17 g/L aquarium salt, 20 g/L glucose, 10 g/L Yeast |
| | extract, 10 g/L Peptone |
| | GY: 17 g/L aquarium salt, 30 g/L glucose, 10 g/L yeast extract |

### Transformation:

Approximately 10 μL of cells were taken off of a plate and resuspended in 1 mL of FM002. 10 μL of this suspension were used to inoculate 50 mL of FM002 in a baffled, 250-mL flask. This culture was incubated in an orbital shaker at 30° C. and 150 rpm. After approximately 20 hours, the mid-growth phase cells were collected (2000×g for 5 min) and suspended in 20 mL 1 M mannitol (pH 5.5) and transferred to a 125-mL, flat-bottom flask. The cells were enzyme treated by addition of 200 μL of 1 M CaCl₂ and 500 μL of 10 mg/mL protease XIV and incubated for 4 hours in an orbital shaker at 30° C. and 100 rpm. Cells were collected in round-bottom tubes and washed with an equal volume of cold 10% glycerol. The cells were then suspended with 4× pellet volume of electroporation buffer. 100 μL of cells were mixed with DNA in a pre-chilled 0.2 cm electroporation cuvette and electroporated (200 Ω, 25 μF, 700 V). Immediately after electroporation, 1 mL of GY medium was added, and cells were transferred to a round-bottom snap-cap tube and recovered over-night at 30° C. and 150 rpm. The recovered cells were then plated onto FM001 supplemented with appropriate antibiotics.

### Example 5

### Fatty Acid Feeding Experiments in Aurantiochytrium

Each gene was heterologously expressed in an Aurantiochytrium PUFA auxotroph strain while co-feeding DHA and the test PUFAs as free fatty acids. GC-FAME analysis of the resulting cultures was used to elucidate enzyme function. Cultures expressing candidate genes were inoculated into FM002 medium supplemented with 1% tergitol solution (type NP-40, 70% in H₂O) and 1 mM DHA. Cultures were incubated for 24 hours at 30° C., 150 rpm, at which time they were amended with 1 mM test PUFA and grown an additional 24 hours. Prior to sampling for GC-FAME analysis, culture pellets were washed to remove residual medium. The activity of each enzyme on a given substrate was measured as the percent of the substrate converted to the product: % conversion=100×product (μg)/[product (μg)+substrate (μg)].

### Example 6

### Expression of Omega-3 Desaturases in S. cerevisiae and Aurantiochytrium

An omega-3 desaturase converts omega-6 fatty acids into omega-3 fatty acids. SEQ ID NOs: 1 and 21-23 are putative omega-3 desaturases (see Table 14 for a description of SEQ ID NOs: 1-26). These enzymes were tested for function and specificity in S. cerevisiae using the feeding experiment described above. The enzymes encoded by all four sequences were capable of converting ARA to EPA (FIG. 3A). SEQ ID NO: 1 was further shown to have a marked preference for the C20 substrates DGLA and ARA, while SEQ ID NO: 21 was shown to have no preference between C18 and C20 substrates (FIG. 3B). The CDS of SEQ ID NO: 1 was subsequently subcloned and expressed in an Aurantiochytrium PUFA auxotroph strain. Activity in this host was dramatically higher than in S. cerevisiae, exceeding 75% substrate conversion of ARA (FIG. 3C). In this background, a slight preference for ARA over DGLA also became apparent.

### Example 7

### Expression of Δ5-Desaturases in S. cerevisiae and Aurantiochytrium

A Δ5 desaturase acts on the C20 omega-6 substrate DGLA and the C20 omega-3 substrate ETA. Three putative Δ5 desaturases (SEQ ID NOs: 2-4) were characterized in S. cerevisiae (FIG. 4A). All three enzymes demonstrated Δ5 desaturase activity in S. cerevisiae, and the enzyme encoded by SEQ ID NO: 2 also exhibited slight Δ8 desaturase activity (the use of EtrA and/or EDA as substrates. EDA=eicosadieneoic acid; ETrA=eicosatrienoic acid). SEQ ID NOs: 2 and 4 were subcloned and expressed in an Aurantiochytrium PUFA auxotroph strain, where they both more than doubled their substrate conversion of DGLA compared to expression in S. cerevisiae (FIG. 4B). Furthermore, the dual specificity of the desaturase encoded by Seq. 2 was also evident in the Aurantiochytrium background (FIG. 4C).

### Example 8

### Expression of Δ6-Elongases in S. cerevisiae and Aurantiochytrium

A Δ6 elongase acts on the C18 omega-6 substrate GLA and the C18 omega-3 substrate SDA. Four putative Δ6 elongases (SEQ ID NOs: 5-8) were characterized in S. cerevisiae (FIG. 5A). All four enzymes demonstrated Δ6 elongase activity in S. cerevisiae, and all also exhibited one or more additional activities. The primary activity of the enzyme encoded by SEQ ID NO: 5 is a Δ9 elongase (which uses LA and/or ALA as substrates) with secondary activity towards Δ6 substrates. SEQ ID NOs: 6 and 7 encode dual-function Δ6/Δ9 elongases with primary activity towards Δ6 elongase substrates. SEQ ID NO: 8 is a tri-functional Δ6/Δ5/Δ9 elongase (a Δ5 elongase acts on the C20 omega-6 substrate ARA and the C20 omega-3 substrate EPA). SEQ ID NO: 5 was subcloned and expressed in an Aurantiochytrium PUFA auxotroph strain, where substrate conversion was improved (FIG. 5B), although substrate specificity remained essentially unchanged.

### Example 9

### Expression of Δ6 Desaturases in S. cerevisiae and Aurantiochytrium

A Δ6 desaturase acts on the C18 omega-6 substrate LA and the C18 omega-3 substrate ALA. Four putative Δ6 desaturases (SEQ ID NOs: 9-12) were characterized in S. cerevisiae. All four enzymes demonstrated Δ6 desaturase activity in S. cerevisiae (FIG. 6A), and SEQ ID NO: 9 also exhibited secondary Δ8 desaturase activity (the use of EDA and ETrA as substrates) (FIG. 6B). No enzyme displayed any preference between omega-3 and omega-6 substrates. SEQ ID NO: 9 was sub-cloned and expressed in an Aurantiochytrium PUFA auxotroph strain, where substrate conversion remained essentially unchanged (FIG. 6C).

### Example 10

### Expression of Δ12 Desaturases in S. cerevisiae and Aurantiochytrium

A Δ12 desaturase acts on the C18:1 substrate OA. SEQ ID NOs: 13 and 24-26 are putative Δ12 desaturases. SEQ ID NO: 13 was tested for function and specificity in S. cerevisiae using the feeding experiment described above. The results showed Δ12 desaturase activity and no secondary activities (FIG. 7A). Subsequently, SEQ ID NOs: 24-26 were identified by their ability to desaturate the host's endogenously produced OA into LA (FIG. 7B) and also encoded no known secondary activities. The CDS of SEQ ID NO: 13 was subsequently subcloned and expressed in an Aurantiochytrium PUFA auxotroph strain. Activity in this host was doubled compared to S. cerevisiae, exceeding 80% substrate conversion of OA (FIG. 7C). In this background, slight omega-3 desaturase activity was detected with LA.

### Example 11

### Expression of a Δ9 Desaturase in S. cerevisiae and Aurantiochytrium

A Δ9 desaturase acts on the C18:0 substrate SA. In S. cerevisiae, OLE1 is an essential gene. The native copy of the S. cerevisiae OLE1 was deleted and simultaneously replaced with a single copy of putative Δ9 desaturases. It was found that SEQ ID NOs: 14 and 15 were able to functionally replace the native OLE1 sequence, suggesting that these genes encode enzymes with Δ9 desaturase activity. SEQ ID NO: 15 was codon optimized for expression in Aurantiochytrium and subcloned for co-expression with a C16 elongase (SEQ ID NO: 16) in Aurantiochytrium. Together, expression of SEQ ID NOs: 15 and 16 lowered C16:0 content, raised C18:0 content, and caused the appearance of OA (FIG. 8).

### Example 12

### Expression of C16 Elongases in S. cerevisiae and Aurantiochytrium

A C16 elongase extends the C16:0 substrate PA to the C18:0 substrate SA. One putative C16 elongase, SEQ ID NO: 17, was characterized in S. cerevisiae. Expression of SEQ ID NO: 17 in this host resulted in depleted C16:0 and elevated C18:0 levels relative to the parental strain (FIG. 9A). SEQ ID NO: 17 was subcloned into a vector carrying additional genes for the elongase/desaturase pathway (see Construct 2 in Example 18 below) and expressed in an Aurantiochytrium PUFA auxotroph strain that also carried Construct 1 (see Example 17 below). A second copy of SEQ ID NO: 17 was independently transformed into this strain, and the resulting FAME analysis revealed a two-fold depletion in C16:0 and fifteen-fold increase in C18:0 compared to the parental strain (FIG. 9B). A second C16 elongase, SEQ ID NO: 16, was codon-optimized for expression in Aurantiochytrium. Expression in Aurantiochytrium alone resulted in minor depletion of C16:0 and elevated C18:0 levels relative to the parental control (FIG. 9C). However, co-expression of SEQ ID NO: 16 in Aurantiochytrium with the Δ9 desaturase encoded by SEQ ID NO: 15 resulted in much greater C16:0 depletion and C18:0 elevation (FIG. 8).

### Example 13

### Expression of Δ5 Elongases in S. cerevisiae

A Δ5 elongase extends the C20 omega-6 substrate ARA and the C20 omega-3 substrate EPA to DTA and DPAn3 (docosapentaenoic acid omega-3), respectively. Two putative Δ5 elongases (SEQ ID NOs: 18 and 19) were characterized in S. cerevisiae (FIG. 10). Both enzymes demonstrated Δ5 elongase activity in S. cerevisiae with additional, minor Δ6 and Δ9 elongase activities.

### Example 14

### Expression of a Δ4 Desaturase in Aurantiochytrium

A Δ4 desaturase modifies the C22 omega-6 substrate DTA and the C22 omega-3 substrate DPAn3 to DPAn6 and DHA, respectively. SEQ ID NO: 20 is a putative M desaturase. This enzyme was tested for function on DPAn3 in an Aurantiochytrium PUFA auxotroph strain using the feeding experiment described above. Results indicated Δ4 desaturase activity (FIG. 11).

### Example 15

### Construct to Convert the C18 Omega-6 Substrate LA and the C18 Omega-3 Substrate ALA to ARA and EPA, Respectively

A subset of characterized elongases and desaturases were chosen to build a complete elongase/desaturase pathway for the production of EPA or ARA in an Aurantiochytrium PUFA auxotroph strain. The pathway enzyme CDSs were divided into two constructs: Construct 1 contains a Δ5 desaturase (SEQ ID NO: 2), a Δ6 elongase (SEQ ID NO: 6), and a Δ6 desaturase (SEQ ID NO: 9). Construct 2 contains the remaining pathway genes (Example 16). Promoters native to the Aurantiochytrium strain were cloned in front of each gene, and a variety of publically available S. cerevisiae terminators were cloned behind each gene. Together, the enzyme CDSs of Construct 1 were linked to a nourseothricin-resistance cassette, and the entire construct was linearized by restriction digest before electroporation into an Aurantiochytrium PUFA auxotroph strain.

### Example 16

### Results Confirming Construct 1

Construct 1 was heterologously expressed in an Aurantiochytrium PUFA auxotroph strain while co-feeding DHA and LA or ALA as free fatty acids. GC-FAME analysis of the resulting cultures was used to evaluate enzyme function. Cultures were inoculated into FM002 medium supplemented with 1% tergitol solution (type NP-40, 70% in H₂O) and 1 mM DHA. Cultures were incubated for 24 hours at 30° C. and 150 rpm, at which time they were amended with 1 mM LA or ALA and grown an additional 24 hours. Prior to sampling for GC-FAME analysis, culture pellets were washed to remove residual medium. The activity of each enzyme on a given substrate was measured as the percent of the substrate converted to the product: % conversion=100×product (μg)/[product (m)+substrate (μg)]. Results from feeding each PUFA confirmed function of Construct 1 on both omega-3 and omega-6 substrates in approximately equal rates (FIG. 12). Minimal accumulation of intermediates is apparent when either LA (FIG. 13A) or ALA (FIG. 13B) is fed. Considerable more ARA and EPA are observed in the experimental stains compared to the control and parent strains.

### Example 17

### Construct to Complete Pathway from C16:0 to EPA

Construct 2 was designed to complement Construct 1 and enable elongation and desaturation of C16:0 (PA) to EPA. Construct 2 contains a Δ12 desaturase (SEQ ID NO: 13), a Δ9 desaturase (SEQ ID NO: 14), a C16 elongase (SEQ ID NO: 17), and an omega-3 desaturase (SEQ ID NO: 1). Promoters native to the Aurantiochytrium strain were cloned in front of each gene, and a variety of publically available S. cerevisiae terminators were cloned behind each gene. Together, the enzyme cassettes were linked to a paromomycin-resistance cassette, and Construct 2 was linearized by restriction digest before electroporation into an Aurantiochytrium PUFA auxotroph strain containing Construct 1.

### Example 18

### Results Confirming Complete Pathway

Construct 2 (containing SEQ ID NOs: 13, 14, 17, and 1) was transformed into an Aurantiochytrium PUFA auxotroph strain containing Construct 1. The resulting transformants were grown in FM002 medium supplemented with 1% tergitol solution (type NP-40, 70% in H₂O) and 1 mM DHA. Prior to sampling for GC-FAME analysis, culture pellets were washed to remove residual medium. Expression of the complete pathway resulted in the appearance of ARA, a PUFA that is not native to the Aurantiochytrium strain used as a host, and an increase in EPA and C18:0 levels (FIG. 14).

### Example 19

### Metabolic Engineering to Improve Elongase and Desaturase Activities in Labyrinthulomycetes Cells

A number of bottlenecks have been identified in the elongase/desaturase EPA pathway, and metabolic pathway engineering strategies have been applied to improve pathway fluxes towards increased production of EPA.

Improvement of Δ6 Desaturase Activity

When Construct 1 (containing SEQ ID NOs: 2, 6, and 9) was transformed into an Aurantiochytrium PUFA auxotroph strain, the resulting strain (Con. 1 in FIG. 15) accumulated ALA when fed this substrate. In Construct 1, the Δ6 desaturase (SEQ ID NO: 9) is under the control of a truncated tubulin alpha chain promoter of the host Aurantiochytrium strain (Tubα-738p) and the PDC1 terminator of S. cerevisiae (PDC1t); the Δ6 elongase (SEQ ID NO: 6) is under the control of a shortened tetraspanin promoter of the host (Tsp-749p) and the ENO2 terminator of S. cerevisiae (ENO2t); and the Δ5 desaturase (SEQ ID NO: 2) is under the control of a shortened mitochondrial chaperonin 60 promoter of the host (Hsp60-788p) and the PGK1 terminator of S. cerevisiae (PGK1t). However, substrate accumulation was substantially reduced and EPA production was increased when an additional copy of SEQ ID NO: 9 was overexpressed in this strain under the control of a much stronger promoter (the full-length tubulin alpha chain promoter, Tubα-997p) and PGK1t (clones 1-4 in FIG. 15). pfaAKO2 is the parental strain for Con. 1; it does not contain any heterologous elo/des genes.

Improvement of C16 Elongase Activity

Tubα-738p drives the expression of the C16 elongase (Seq. 17) in Construct 2. When this construct was transformed into an Aurantiochytrium PUFA auxotroph strain (Ex. 20) that also contained Construct 1, substantial accumulation of C16:0 was observed (Con. 1+2 in FIG. 16). C16:0 accumulation was reduced and C18:0 production increased when an additional copy of Seq. 17 was expressed in this strain under the control of a much stronger promoter (Tubα-997p) and PGK1t (clones 1-15 in FIG. 16).

Improvement Δ9 Desaturase Activity

Despite the step-change improvement in conversion of endogenous C16:0 to C18:0 by overexpression of SEQ ID NO: 17, pathway flux appeared to constrict at C18:0 (FIG. 16). This result indicates that the expression of the Δ9 desaturase (SEQ ID NO: 14) in Construct 2 (under Tsp-749p) might also be sub-optimal. Therefore, this promoter was replaced with the shortened RPL11 promoter from the host (RPL11-699p). Construct 3 (which harbors SEQ ID NO: 17 under Tubα-997p, SEQ ID NO: 14 under RPL11-699p, and a selectable marker) was transformed into a strain containing Constructs 1 and 2. Nine clones of this new strain accumulated higher levels of C18:2 compared to those of the controls (FIG. 17).

Construction of the Second-Generation Pathway Constructs

Based on the findings from different bottlenecks in the pathway and the improved results from pathway optimization, second-generation constructs were built. A second-generation construct (Construct 6) harboring CDSs for the C16 elongase (SEQ ID NO: 17), the Δ9 desaturase (SEQ ID NO: 14), and the Δ12 desaturase (SEQ ID NO: 13) under the control of improved promoters and terminators was built. SEQ ID NO: 17 is under the control of Tubα-997p and the SV40 terminator of Simian virus 40 (SV40t); SEQ ID NO: 14 is under the control of RPL11-699p and ENO2t; and SEQ ID NO: 13 is under the control of Hsp60-788p and PGK1t. A second-generation construct (Construct 7) harboring a hygromycin-resistance cassette, the Δ6 desaturase (SEQ ID NO: 9), the Δ6 elongase (SEQ ID NO: 6), and the Δ5 desaturase (SEQ ID NO: 2) under the control of improved promoters and terminators was built. SEQ ID NO: 9 is under the control of Tubα-997p and SV40t; SEQ ID NO: 6 is under the control of RPL11-699p and ENO2t; and SEQ ID NO: 2 is under the control of Hsp60-788p and PGK1t.

Expression of the Second-Generation Pathway Constructs

The second-generation constructs were expressed in an Aurantiochytrium PUFA auxotroph strain. These new constructs exhibited improvements over the first-generation constructs in terms of both substrate accumulation and final product formation (FIG. 18).

### Example 20

### Strain GH-06701: Inactivation of PUFA PKS by Creating a Homozygous Deletion within pfaA; Cells Require PUFA Supplementation for Growth

Strain GH-06701 was constructed by allelic replacement using homologous recombination, negative selections, and Cre/Lox technology. Both pfaA alleles of Aurantiochytrium strain were inactivated by homologous recombination; deletion cassettes contained: 1) positive selection markers (either nptII—Paromomycin^{r} or hph—Hygromycin^{r}) flanked by loxP sites; and 2) homologous DNA regions designed to delete a portion of the pfaA CDS upon insertion of the cassette into the pfaA locus. During transformation of deletion cassettes the medium was supplemented with 1 mM DHA. After two rounds of transformation, using the nptII deletion cassette followed by the hph deletion cassette, clones were streaked onto solid growth medium with or without DHA. PUFA auxotrophs were obtained; this phenotype is consistent with inactivation of endogenous DHA production from the PKS, mediated by inactivation of pfaA. To remove the nptII and hph markers flanked by loxP sites, a Cre recombinase cassette was introduced into the pfaA deletion strain that contained Cre recombinase linked to both positive (ble—bleocin^{r}) and negative (amdS—fluoracetemide^{s}) selection markers. Upon transformation of the marker removal cassette, bleocin resistant clones were screened for sensitivity to Paromomycin (nptII) and Hygromycin (hph); numerous clones were obtained that were resistant to bleocin and sensitive to Paromomycin and Hygromycin. The Cre recombinase cassette was removed using allelic replacement by transforming a DNA molecule with sequences that flank the Cre recombinase cassette. Numerous transformants were obtained after plating on fluoroacetamide containing medium to select for loss of the amdS containing Cre cassette. Molecular diagnostics, using PCR, was performed on the fluoroacetamide resistant clones to confirm allelic replacement at both alleles of pfaA and removal of the Cre cassette. Strain GH-06701 was one of the positive clones generated from the above process. This strain is a pfaA double knock-out that does not have a functioning PKS pathway and does not produce DHA. It requires supplementation with DHA for growth.

### Example 21

### Strain GH-07655: Conversion of LA to ARA or ALA to EPA; Requires PUFA Supplementation

Elongases and desaturases were chosen to build a complete elongase/desaturase pathway for the production of EPA or ARA in an Aurantiochytrium PUFA auxotroph strain (GH-06701). The pathway enzyme CDSs were divided into three constructs (Table 3): Construct 1 (pW70) contains a Δ5 desaturase (SEQ ID NO: 2), a Δ6 elongase (SEQ ID NO: 6), and a Δ6 desaturase (SEQ ID NO: 9); these activities enable conversion of LA to ARA or ALA to EPA. Each gene is expressed from the promoter and terminators indicated in Table 3; the promoters used are native to the Aurantiochytrium host and the terminators are derived from S. cerevisiae. Transformation of strain GH-06701 with linearized pW70 was selected by plating on hygromycin to select for the hph-containing cassette.

Clones were screened by co-feeding DHA and LA or ALA as free fatty acids. GC-FAME analysis of the resulting cultures was used to evaluate enzyme function. Cultures were inoculated into FM002 medium supplemented with 1% tergitol solution (type NP-40, 70% in H₂O) and 1 mM DHA. Cultures were incubated for 24 hours at 30° C. and 225 rpm, at which time they were amended with 1 mM LA or ALA and grown an additional 24 hours. Prior to sampling for GC-FAME analysis, culture pellets were washed to remove residual medium. The FAME profiles of GH-07655 shown in FIG. 19 demonstrate the successful conversion of LA to ARA and ALA to EPA.

TABLE 3

| Construct | promoter | gene | SEQ ID NO | terminator |
|---|---|---|---|---|
| Construct 1 | LoxP-sctp | hph | — | cyc1t-LoxP |
| pW70 | hsp60sp | Δ5des | 2 | pgk1t |
| | rpl11sp | Δ6elo | 6 | eno2t |
| | tubαp | Δ6des | 9 | sv40t |
| Construct 2 | LoxP-sctp | nptII | — | cyc1t-LoxP |
| pW68 | hsp60sp | Δ12des | 13 | pgk1t |
| | rpl11sp | Δ9des | 14 | eno2t |
| | tubαp | C16elo | 17 | sv40t |
| Construct 3 | LoxP-sctp | nat | — | cyc1t-LoxP |
| pW99 | hsp60sp | Δ12des | 13 | pgk1t |
| | actsp | Δ9des | 14 | eno2t |
| | tubαp | ωdes | 23 | sv40t |

### Example 22

### Strain 1-6-1-82: Conversion of Glucose to ARA; Cells Require PUFA Supplementation for Growth

Based on single enzyme expressions studies in yeast and Labyrinthulomycetes, a subset of elongases and desaturases were chosen to build a complete elongase/desaturase pathway for the production of EPA or ARA in an Aurantiochytrium PUFA auxotroph strain GH-06701. The pathway enzyme CDSs were divided into three constructs (Table 3): pW68 contains a Δ12 desaturase (SEQ ID NO: 13), a Δ9 desaturase (SEQ ID NO: 14), and a C16 elongase (SEQ ID NO: 17); these activities enable conversion of PA to LA. Each gene is expressed from the promoter and terminators indicated in Table 3; the promoters used are native to the Aurantiochytrium host and the terminators are derived from S. cerevisiae. Transformation of GH-07655 with linearized pW68 was selected by plating on Paromomycin to select for the nptII-containing cassette.

Clones were screened by co-feeding DHA as free fatty acids. GC-FAME analysis of the resulting cultures was used to evaluate enzyme function. Cultures were inoculated into FM2 medium supplemented with 1% tergitol solution (type NP-40, 70% in H₂O) and 1 mM DHA. Cultures were incubated for 24 hours at 30° C. and 225 rpm. Prior to sampling for GC-FAME analysis, culture pellets were washed to remove residual medium. The FAME profile of clone 1-6-1-82 in FIG. 20 shows the successful conversion of PA into ARA. Despite the production of about 9% ARA, these strains still required DHA supplementation for growth and the high DHA levels are from the exogenous feeding of this fatty acid. Additional PUFA dependent clones generated in this manner were 1-6-2-20, 1-6-2-33, 1-6-2-95, and 1-6-3-33.

### Example 23

### Strain GH-SGI-7990: Conversion of Glucose to ARA; PUFA Supplementation not Required

The advantages of this strain include the ability to produce non-DHA lipid compositions including microbial oils and biomass, a simplified process, and reduced product costs. Restoring PUFA prototrophy and robustness was achieved by serial transfer in medium lacking PUFA supplementation as described in the paragraph below.

Clones 1-6-1-82, 1-6-2-20, 1-6-2-33, 1-6-2-95, and 1-6-3-33 were each inoculated into 3 mL of FM002 medium containing 1% tergitol and 1 mM DHA and grown overnight at a shake speed of 225 rpm at 30° C. The overnight cultures were each back-diluted into FM002 medium (1 mL into 25 mL) and allowed to grow without DHA for 3 days. Growth was visibly improved for all five clones at the end of the 3-day fermentation. The cultures were back-diluted again into fresh FM002 medium and allowed to grow for another 3 days; growth appeared to be significantly improved. Culture samples were submitted for FAME and total organic carbon (TOC) analyzes. This first set of PUFA-independent (prototrophs) clones were cryopreserved as GH-07917 to GH-07921, respectively. Two more sets of clones were generated by back-diluting two additional times in the same manner. The second set of clones were cryopreserved as GH-07995 to GH-07999 and the third set was cryopreserved as GH-07990 to GH-07994. The FAME and TOC analysis performed on these claims are found in Table 4.

TABLE 4
FAME/TOC profiles of PUFA independent ARA Strains

| | | % FAME | | | | | | | FAME/TOC | Total | TOC |
|---|---|---|---|---|---|---|---|---|---|---|---|
| ID | Strain | PA | SA | OA | LA | GLA | DGLA | ARA | (%) | FAME(μg) | (μg) |
| 07917 | 1-6-1-82 | 20.0 | 24.4 | 1.3 | 1.2 | 1.8 | 1.9 | 35.9 | 40.0 | 1095.57 | 2738.00 |
| 07918 | 1-6-2-20 | 19.2 | 10.6 | 2.0 | 2.9 | 1.8 | 2.7 | 32.8 | 46.5 | 1428.64 | 3072.67 |
| 07919 | 1-6-2-33 | 27.4 | 13.1 | 2.4 | 3.4 | 2.4 | 4.1 | 30.7 | 58.0 | 1095.80 | 1888.00 |
| 07920 | 1-6-2-95 | 20.8 | 11.8 | 1.3 | 1.8 | 2.1 | 2.8 | 41.2 | 63.2 | 972.62 | 1540.00 |
| 07921 | 1-6-3-33 | 16.7 | 9.8 | 0.8 | 4.0 | 2.7 | 6.0 | 40.4 | 40.6 | 617.90 | 1520.67 |
| 07995 | 1-6-1-82 | 18.8 | 32.9 | 1.6 | 0.7 | 1.3 | 2.2 | 27.8 | 24.7 | 403.54 | 1636.00 |
| 07996 | 1-6-2-20 | 18.5 | 11.9 | 1.6 | 1.1 | 1.4 | 2.7 | 42.6 | 24.1 | 581.17 | 2409.33 |
| 07997 | 1-6-2-33 | 17.6 | 17.8 | 3.8 | 4.4 | 3.6 | 6.5 | 23.7 | 57.9 | 1488.84 | 2569.33 |
| 07998 | 1-6-2-95 | 21.2 | 10.2 | 1.6 | 1.7 | 2.0 | 2.7 | 41.8 | 42.6 | 629.31 | 1476.00 |
| 07999 | 1-6-3-33 | 19.7 | 11.4 | 1.7 | 5.1 | 2.8 | 6.4 | 37.4 | 36.3 | 589.80 | 1624.00 |
| 07990 | 1-6-1-82 | 22.0 | 27.8 | 2.5 | 1.8 | 2.4 | 2.8 | 27.3 | 57.1 | 1901.09 | 3229.33 |
| 07991 | 1-6-2-20 | 25.5 | 19.7 | 6.7 | 3.6 | 1.8 | 4.1 | 22.9 | 61.4 | 1619.90 | 2637.33 |
| 07992 | 1-6-2-33 | 14.6 | 11.3 | 3.3 | 3.4 | 4.1 | 7.2 | 38.2 | 26.0 | 714.14 | 2745.33 |
| 07993 | 1-6-2-95 | 19.2 | 10.5 | 1.7 | 1.9 | 2.6 | 3.0 | 45.9 | 39.1 | 882.70 | 2258.67 |
| 07994 | 1-6-3-33 | 19.6 | 8.9 | 1.4 | 3.2 | 3.0 | 5.9 | 46.6 | 31.0 | 572.90 | 1849.33 |

### Example 24

### Strain GH-08962: Conversion of Glucose to ARA; PUFA Supplementation not Required

The following example shows restoration of PUFA prototrophy and improved growth rates. Clone 1-6-1-82 producing up to 9% ARA/FAME when supplemented with 1 mM DHA was adapted to grow on ARA over a period of 1 week with obvious improvements in growth rate evident. This clone was passaged three times in FM002 medium containing 0.5% tergitol and 1 mM ARA; growth was visibly improved over time. Following adaptation, the culture was inoculated into FM2 medium without PUFA and was able to grow. The culture was preserved as GH-07832 and samples were submitted for FAME and TOC analyzes (Table 5). The FAME profile of GH-07832 differed in the absence of any PUFA supplementation, compared to that of its parent, with ARA/FAME close to 37% during growth and 23.7% at 96 hours.

TABLE 5
GH-07832 Growth without PUFA

| | | | | | C16:2 | | | C18:1 |
|---|---|---|---|---|---|---|---|---|
| | Hrs | C14:0 | C15:0 | C16:0 | Cis7, 10? | C17:0 | C18:0 | Cis6 + 7 + 8 + 9 |
| μg/ml | 24 | 0.0 | 0.0 | 11.5 | 0.0 | 0.0 | 4.9 | 5.6 |
| % FAME | 96 | 11.5 | 13.5 | 229.5 | 5.7 | 18.9 | 149.0 | 30.3 |
| | 24 | 0.0% | 0.0% | 25.6% | 0.0% | 0.0% | 10.8% | 12.4% |
| | 96 | 1.6% | 1.9% | 32.8% | 0.8% | 2.7% | 21.3% | 4.3% |

| | | C18:2 | C18:3 | C20:3 | | | | |
|---|---|---|---|---|---|---|---|---|
| | Hrs | Cis9, 12 | Cis6, 9, 12 | Cis8, 11, 14 | ARA | EPA | FAME | TOC |
| μg/ml | 24 | 2.6 | 1.6 | 2.3 | 16.6 | 0.0 | 45.1 | 286.6 |
| % FAME | 96 | 25.7 | 22.8 | 17.5 | 165.7 | 10.3 | 700.3 | 2215.2 |
| | 24 | 5.9% | 3.5% | 5.0% | 36.9% | 0.0% | 100.0% | 15.7% |
| | 96 | 3.7% | 3.3% | 2.5% | 23.7% | 1.5% | 100.0% | 31.6% |

While strain GH-07832 was a good ARA producer, it has opportunity for growth improvement through classical strain development: (1) it grows significantly slower than its DHA-producing parent (approximate division rate in FM002 medium is every 3-4 hours) and (2) it is even slower in minimal medium without yeast extract and peptone (approximate division rate is every 5 hours). Due to these issues, this new strain GH-07832 was grown up in FM002 medium and inoculated into a continuous culture apparatus (automated flow cytometry system) with minimal DHA production medium (version 1). Growth during the first few days was slow (˜0.2) but did gradually increase to ˜0.25 where it remained during the last 3 days of the 10-day fermentation. At the end of the fermentation, cells from the cytostat were sub-cultured in shake flasks. Growth in minimal medium was compared between the cytostat culture and a culture that had been sub-cultured in FM002 and then grown up in minimal medium. Two sequential cultures revealed that the cytostat culture had a 28.8% increase in growth rate in the minimal medium (0.268 vs. 0.208). The endpoint cytostat culture was cryopreserved as GH-08034. Single colony isolates were generated from this culture yielding strain GH-08962.

### Example 25

### Strain GH-13080: Conversion of Glucose to EPA, PUFA Supplementation not Required for Cell Growth

This is an example of an engineered Labyrinthulomycetes cell producing EPA from a sugar using the elongase and desaturase pathway.

The pathway enzyme CDSs were divided into three constructs (Table 3): Construct 3 (pW99) contains a Δ12 desaturase (SEQ ID NO: 13), a Δ9 desaturase (SEQ ID NO: 14), and a ω3 desaturase (SEQ ID NO: 23). Each gene is expressed from the promoter and terminators indicated in Table 3; the promoters used are native to the Aurantiochytrium host and the terminators are derived from S. cerevisiae. GH-13080 was generated by transforming ARA-producing GH-08962 with linearized pW99; selection on Nourseothricin was followed by PCR to confirm the presence of the ω3 desaturase. The introduction of the ω3 desaturase will convert the ARA-producing host into an EPA producing strain; the ω3 desaturase converts ARA to EPA.

GH-13080 was inoculated in 3 mL of FM002 medium and grown for 72 hr at 30° C. and 225 rpm before submitting the culture for FAME analysis. The FAME profiles (FIG. 21) clearly show that addition of ω3 desaturase leads to EPA production.

### Example 26

### Fermentation Media

Shake flask medium was prepared by dissolving medium components in water and adjusted to pH to 5.8 with sodium hydroxide. The medium can be filter-sterilized or autoclaved for 45 minutes at 121.1° C. Post heat sterilization: dextrose, MES buffer, magnesium sulfate, trace element solution and vitamins are added aseptically to the shake flask medium.

Production fermenter medium was prepared by dissolving medium components in water and adjusted to pH to 5.8 with sodium hydroxide. The medium can be filter-sterilized or autoclaved for 45 minutes at 121.1° C. Post heat sterilization vitamins are added aseptically to the production medium.

### Example 27

### 2 L Fermentation Process

The purpose of the shake flask fermentation is to grow cell mass to inoculate the production fermenter. Vessels containing shake flask medium were inoculated with cryogenically preserved cells and incubated at 30° C., 150 RPM until the optical density at a wavelength of 740 nm (OD₇₄₀) reached a value between 3 and 8.

A production fermenter containing production medium (Table 6) is inoculated with culture from the shake flask stage. The production fermentation has two phases: 1) a growth phase to increase cell density; and 2) a lipid phase to increase the lipid content.

For the growth phase, the production fermenter is operated at the optimum growth conditions until the culture reaches the desired biomass wet cell weight (WCW) that ranges from 160 to 180 g_WCW/L. A concentrated dextrose feed with nutrients was started to keep the dextrose concentration between 15 to 25 g/L. Residual dextrose concentrations are kept between 15 to 25 g/L. During the growth phase the pH is maintained at 6.3 using 30% ammonium hydroxide or ammonia (pure gas). The base also provides a majority of the nitrogen that is required for the cells to grow.

The lipid phase was induced by limiting nitrogen. This limitation was achieved by substituting the base (ammonium hydroxide or ammonia) with 45% potassium hydroxide. The production fermenter was operated at the optimum fermentation conditions for lipid accumulation until all the dextrose and co-feed is added to the fermentation. The dextrose concentration was kept between 15 to 25 g/L to supply the cells with sufficient dextrose for lipid accumulation.

TABLE 6
Production Medium Composition

| Medium Components | Final Concentration (g/L) |
|---|---|
| Potassium Phosphate Monobasic (KH₂PO₄) | 3.00 |
| Potassium Chloride (KCl) | 0.50 |
| Magnesium Chloride (MgCl₂•6H₂O) | 5.00 |
| Sodium EDTA-2H20 (Na₂EDTA•2H₂O) | 0.20 |
| Boric Acid (H₂BO₃) | 0.07 |
| Iron Chloride (FeCl₂•4H₂O) | 0.05 |
| Cobalt Chloride (CoCl₂•6H₂O) | 0.07 |
| Manganese Chloride (MnCl₂•4H₂O) | 0.009 |
| Zinc Chloride (ZnCl₂) | 0.03 |
| Nickel Sulfate (NiSO₄•6H₂O) | 0.007 |
| Copper Sulfate (CuSO₄•5H₂O) | 0.002 |
| Sodium Molybdenate (Na₂MoO₄•2H₂O) | 0.021 |
| Vitamin B12 | 0.000002 |
| Biotin | 0.000002 |
| Thiamine | 0.0004 |

### Example 28

### Phylogeny of Strain WH-5628

Utilizing organisms isolated per Example 1 three genetic loci, 18SrDNA, actin, and β-tubulin, were studied to establish a phylogenetic tree as per Tsui et al. (Molecular Phylogenetics and Evolution 50: 129-140 (2007)). All thraustochytrid reference genera were included in the analysis with the exception of Biocosoeca sp. and Caecitellus sp. For each locus, four methods of tree construction were performed: maximum likelihood, maximum parsimony, minimum evolution, and neighbor joining. Based on the results the closest relative of the WH-5628 strain appears to be Aurantiochytrium mangrovei (basionym: Schizochytrium mangrovei). Schizochytrium sp. ATCC 20888 is also closely related although not as closely related as Aurantiochytrium mangrovei. Based on the barcoding gap differential for the three genetic loci WH-5628 is indicated to be an Aurantiochytrium species.

Lipid profiles (Example 29) of WH-5628 confirm this. Yokoyama and Honda (Mycoscience 48: 199-211 (2007)) define Aurantiochytrium species as having 5% or less of FAME lipids as arachidonic acid (ARA), and up to about 80% of FAME lipids as DHA. In contrast, Schizochytrium species have an ARA content of about 20% FAME.

In addition, analysis of the carotenoids of strain WH-5628 demonstrated that the strain produces the carotenoids echinenone, canthaxanthin, phoenicoxanthin, and astaxanthin, which are characteristic of Aurantiochytrium species but lacking in Schizochytrium species (Yokoyama and Honda (2007)).

Finally, strain WH-5628 were observed microscopically during vegetative growth. Consistent with the morphological description of Aurantiochytrium by Yokoyama and Honda (2007), vegetative cells of WH-5628 were found to be dispersed as single cells and were not found in the large aggregates characteristic of the Schizochytrium. Cultures propagated in liquid medium at 15° C. were visibly pigmented after propagation for 60 hours, a phenotype consistent with the identification of WH-5628 as Aurantiochytrium and not Schizochytrium. Additional and suitable Larynthulomycetes strains are also available from ATCC.

### Example 29

### Fermentation Profile of Strain WH-5628; Aurantiochytrium Producing DHA and Minor Amounts of EPA or ARA

This example shows an end of fermentation profile of the fatty acid profile obtained with a fermentation process of the present invention. A 2 L scale fed-batch experiment was conducted using a procedure similar to the previous example. The PUFA profile of WH-5628 shows a large amount of DHA (about 30%) but small amounts of ARA (less than about 0.6%) and EPA (less than 0.2%) (Table 7).

TABLE 7
Fatty Acid Composition Obtained in 2
L Fermentation With Strain WH-5628

| Fatty Acid | Titer (g/L) | % Total FAME |
|---|---|---|
| C14:0 | 4.44 | 4.17% |
| C14:1 cis9 | 0.00 | 0.00% |
| C15:0 | 0.39 | 0.37% |
| C15:1 cis10 | 0.00 | 0.00% |
| C16:0 | 58.45 | 54.83% |
| C16:1 cis6 + 7 | 0.00 | 0.00% |
| C16:1 cis9 | 0.12 | 0.11% |
| C16:1 cis11 | 0.00 | 0.00% |
| C16:2 cis7, 10 | 0.00 | 0.00% |
| C16:2 cis9, 12 | 0.00 | 0.00% |
| C16:3 cis4, 7, 10 | 0.00 | 0.00% |
| C17:0 | 0.00 | 0.00% |
| C16:3 cis6, 9, 12 | 0.00 | 0.00% |
| C16:3 cis7, 10, 13 | 0.00 | 0.00% |
| C17:1 cis10 | 0.00 | 0.00% |
| C16:4 cis4, 7, 10, 13 | 0.00 | 0.00% |
| C16:4 cis6, 9, 12, 15 | 0.00 | 0.00% |
| C18:0 (SA) | 2.02 | 1.89% |
| C18:1 cis6 + 7 + 8 + 9 | 0.05 | 0.05% |
| C18:1 cis11 | 0.00 | 0.00% |
| C18:1 cis12 + C18:2 cis5, 9 | 0.00 | 0.00% |
| C18:2 cis6, 9 | 0.00 | 0.00% |
| C18:2 cis9, 12 | 0.00 | 0.00% |
| C18:2 trans9, 12 | 0.00 | 0.00% |
| C18:3 cis6, 9, 12 | 0.00 | 0.00% |
| C19:0 | 0.00 | 0.00% |
| C18:3 cis8, 11, 14 | 0.00 | 0.00% |
| C18:3 cis9, 12, 15 | 0.00 | 0.00% |
| C18:4 cis6, 9, 12, 15 | 0.00 | 0.00% |
| C18:2 cis9, 11 | 0.00 | 0.00% |
| C20:0 | 0.46 | 0.43% |
| C20:1 cis11 | 0.00 | 0.00% |
| C20:2 cis11, 14 | 0.00 | 0.00% |
| C20:3 cis8, 11, 14 | 0.00 | 0.00% |
| C21:0 | 0.00 | 0.00% |
| C20:4 cis5, 8, 11, 14 (ARA) | 0.59 | 0.55% |
| C20:3 cis11, 14, 17 | 0.00 | 0.00% |
| C20:4 cis8, 11, 14, 17 | 0.50 | 0.47% |
| C20:5 cis5, 8, 11, 14, 17 (EPA) | 0.15 | 0.14% |
| C22:0 | 0.00 | 0.00% |
| C22:1 cis13 | 0.00 | 0.00% |
| C22:2 cis13, 16 | 0.00 | 0.00% |
| C22:4 cis7, 10, 13, 16 | 0.00 | 0.00% |
| C22:3 cis13, 16, 19 | 0.00 | 0.00% |
| C22:5 cis4, 7, 10, 13, 16 | 7.88 | 7.39% |
| C22:5 cis7, 10, 13, 16, 19 | 0.00 | 0.00% |
| C24:0 | 0.00 | 0.00% |
| C22:6 (DHA) | 31.54 | 29.59% |
| C24:1 | 0.00 | 0.00% |
| Total FAME | 106.60 | 100.00% |

### Example 30

### 2 L Fermentation Profile of Strain GH-7990; Cell Producing ARA and Little or No EPA or DHA

This example shows an end of fermentation profile of the fatty acid profile obtained with a fermentation process of the present invention. A 2 L scale fed-batch experiment was conducted using a procedure similar to Example 27. The PUFA profile of GH-7990 shows a small amount of DHA (˜1%) and EPA (<1.5%), and considerable ARA (>15%). Some characteristics of the fermentation are indicated in Table 8. The saturated fatty acid profile of GH-7990 also shows the strain accumulating >26% SA (C18:0) (Table 9).

TABLE 8
Fermentation performance of GH-07990 at 2-L Scale

| ARA (g/L) | ARA (% FAME) | TFA % DCW | TFA (g/L) |
|---|---|---|---|
| 4.0 | 15.9 | 35.6 | 24.7 |

TABLE 9
Fatty Acid Composition Obtained in 2 L Fermentation
Performed With Strain GH-07990

| Fatty Acid | Titer (g/L) | % Total FAME |
|---|---|---|
| C14:0 | 0.23 | 0.94% |
| C14:1 cis9 | 0.00 | 0.00% |
| C15:0 | 0.17 | 0.67% |
| C15:1 cis10 | 0.00 | 0.00% |
| C16:0 | 6.56 | 26.50% |
| C16:1 cis6 + 7 | 0.00 | 0.00% |
| C16:1 cis9 | 0.00 | 0.00% |
| C16:1 cis11 | 0.00 | 0.00% |
| C16:2 cis7, 10 | 0.18 | 0.71% |
| C16:2 cis9, 12 | 0.00 | 0.00% |
| C16:3 cis4, 7, 10 | 0.00 | 0.00% |
| C17:0 | 0.32 | 1.28% |
| C16:3 cis6, 9, 12 | 0.00 | 0.00% |
| C16:3 cis7, 10, 13 | 0.00 | 0.00% |
| C17:1 cis10 | 0.00 | 0.00% |
| C16:4 cis4, 7, 10, 13 | 0.00 | 0.00% |
| C16:4 cis6, 9, 12, 15 | 0.00 | 0.00% |
| C18:0 (SA) | 6.62 | 26.74% |
| C18:1 cis6 + 7 + 8 + 9 | 1.41 | 5.70% |
| C18:1 cis11 | 0.26 | 1.04% |
| C18:1 cis12 + C18:2 cis5, 9 | 0.00 | 0.00% |
| C18:2 cis6, 9 | 0.00 | 0.00% |
| C18:2 cis9, 12 | 1.79 | 7.25% |
| C18:2 trans9, 12 | 0.00 | 0.00% |
| C18:3 cis6, 9, 12 | 1.40 | 5.64% |
| C19:0 | 0.00 | 0.00% |
| C18:3 cis8, 11, 14 | 0.00 | 0.00% |
| C18:3 cis9, 12, 15 | 0.16 | 0.65% |
| C18:4 cis6, 9, 12, 15 | 0.07 | 0.28% |
| C18:2 cis9, 11 | 0.00 | 0.00% |
| C20:0 | 0.25 | 1.01% |
| C20:1 cis11 | 0.07 | 0.29% |
| C20:2 cis11, 14 | 0.00 | 0.00% |
| C20:3 cis8, 11, 14 | 0.47 | 1.90% |
| C21:0 | 0.00 | 0.00% |
| C20:4 cis5, 8, 11, 14 (ARA) | 3.95 | 15.96% |
| C20:3 cis11, 14, 17 | 0.00 | 0.00% |
| C20:4 cis8, 11, 14, 17 | 0.00 | 0.00% |
| C20:5 cis5, 8, 11, 14, 17 (EPA) | 0.32 | 1.28% |
| C22:0 | 0.20 | 0.81% |
| C22:1 cis13 | 0.00 | 0.00% |
| C22:2 cis13, 16 | 0.00 | 0.00% |
| C22:4 cis7, 10, 13, 16 | 0.07 | 0.29% |
| C22:3 cis13, 16, 19 | 0.00 | 0.00% |
| C22:5 cis4, 7, 10, 13, 16 | 0.00 | 0.00% |
| C22:5 cis7, 10, 13, 16, 19 | 0.00 | 0.00% |
| C24:0 | 0.00 | 0.00% |
| C22:6 (DHA) | 0.26 | 1.03% |
| C24:1 | 0.00 | 0.00% |
| Total FAME | 24.74 | 100.00% |

### Example 31

### 2 L Fermentation Profile of Strain GH-08962; Labyrinthulomycete Producing ARA and No EPA or DHA

This example shows an end of fermentation profile of the fatty acid profile obtained with a fermentation process of the present invention. A 2 L scale fed-batch experiment was conducted using a procedure similar to Example 27. The PUFA profile of GH-08962 is unique for a Labyrinthulomycetes strain; it shows no DHA, a small amount of EPA (<0.5%), and considerable ARA (14%). Some characteristics of the fermentation are indicated in Table 10. The saturated fatty acid profile of GH-08962, accumulating >32% SA, is also unique for a Labyrinthulomycetes strain (Table 11).

TABLE 10
Fermentation performance of GH-08962 at 2 L scale

| ARA (g/L) | ARA (% FAME) | TFA % DCW | TFA (g/L) |
|---|---|---|---|
| 5.8 | 14.0 | 45.9 | 41.6 |

TABLE 11
Fatty acid composition obtained in 2 L Fermentation
performed with strain GH-08962

| Fatty Acid | Titer (g/L) | Total FAME (%) |
|---|---|---|
| C14:0 | 0.48 | 1.14% |
| C14:1 cis9 | 0.00 | 0.00% |
| C15:0 | 0.52 | 1.24% |
| C15:1 cis10 | 0.00 | 0.00% |
| C16:0 | 12.70 | 30.52% |
| C16:1 cis6 + 7 | 0.00 | 0.00% |
| C16:1 cis9 | 0.00 | 0.00% |
| C16:1 cis11 | 0.00 | 0.00% |
| C16:2 cis7, 10 | 0.07 | 0.17% |
| C16:2 cis9, 12 | 0.00 | 0.00% |
| C16:3 cis4, 7, 10 | 0.00 | 0.00% |
| C17:0 | 0.96 | 2.30% |
| C16:3 cis6, 9, 12 | 0.00 | 0.00% |
| C16:3 cis7, 10, 13 | 0.00 | 0.00% |
| C17:1 cis10 | 0.00 | 0.00% |
| C16:4 cis4, 7, 10, 13 | 0.00 | 0.00% |
| C16:4 cis6, 9, 12, 15 | 0.00 | 0.00% |
| C18:0 (SA) | 13.59 | 32.66% |
| C18:1 cis6 + 7 + 8 + 9 | 1.43 | 3.45% |
| C18:1 cis11 | 0.00 | 0.00% |
| C18:1 cis12 + C18:2 cis5, 9 | 0.00 | 0.00% |
| C18:2 cis6, 9 | 0.00 | 0.00% |
| C18:2 cis9, 12 | 2.27 | 5.46% |
| C18:2 trans9, 12 | 0.00 | 0.00% |
| C18:3 cis6, 9, 12 | 2.05 | 4.93% |
| C19:0 | 0.00 | 0.00% |
| C18:3 cis8, 11, 14 | 0.00 | 0.00% |
| C18:3 cis9, 12, 15 | 0.00 | 0.00% |
| C18:4 cis6, 9, 12, 15 | 0.00 | 0.00% |
| C18:2 cis9, 11 | 0.00 | 0.00% |
| C20:0 | 0.32 | 0.78% |
| C20:1 cis11 | 0.00 | 0.00% |
| C20:2 cis11, 14 | 0.00 | 0.00% |
| C20:3 cis8, 11, 14 | 0.83 | 2.00% |
| C21:0 | 0.00 | 0.00% |
| C20:4 cis5, 8, 11, 14 (ARA) | 5.83 | 14.00% |
| C20:3 cis11, 14, 17 | 0.00 | 0.00% |
| C20:4 cis8, 11, 14, 17 | 0.00 | 0.00% |
| C20:5 cis5, 8, 11, 14, 17 (EPA) | 0.19 | 0.47% |
| C22:0 | 0.17 | 0.42% |
| C22:1 cis13 | 0.00 | 0.00% |
| C22:2 cis13, 16 | 0.00 | 0.00% |
| C22:4 cis7, 10, 13, 16 | 0.20 | 0.48% |
| C22:3 cis13, 16, 19 | 0.00 | 0.00% |
| C22:5 cis4, 7, 10, 13, 16 | 0.00 | 0.00% |
| C22:5 cis7, 10, 13, 16, 19 | 0.00 | 0.00% |
| C24:0 | 0.00 | 0.00% |
| C22:6 (DHA) | 0.00 | 0.00% |
| C24:1 | 0.00 | 0.00% |
| Total FAME | 41.63 | 100.00% |

### Example 32

### 2 L Fermentation Profile of Strain GH-13080; Labyrinthulomycete Producing EPA and No DHA

This example shows an end of fermentation profile of the fatty acid profile obtained with a fermentation process of the present invention. A 2 L scale fed-batch experiment was conducted using a procedure similar to Example 27. The PUFA profile of GH-13080 is unique for a Labyrinthulomycetes strain; it shows no MIA, and considerable EPA (>11%). Some characteristics of the fermentation are indicated in Table 12. The saturated fatty acid profile of GH-13080, accumulating >23% OA, is also unique for a Labyrinthulomycetes strain (Table 13).

TABLE 12
Fermentation performance of GH-13080 at 2 L scale

| EPA (g/L) | EPA (% FAME) | TFA % DCW | TFA (g/L) |
|---|---|---|---|
| 4.5 | 11.2 | 59.0 | 39.9 |

TABLE 13
Fatty Acid Composition Obtained in 2 L Fermentation
performed with strain GH-13080

| Fatty Acid | Titer (g/L) | Total FAME (%) |
|---|---|---|
| C14:0 | 0.36 | 0.90% |
| C14:1 cis9 | 0.00 | 0.00% |
| C15:0 | 0.07 | 0.18% |
| C15:1 cis10 | 0.00 | 0.00% |
| C16:0 | 9.22 | 23.09% |
| C16:1 cis6 + 7 | 0.00 | 0.00% |
| C16:1 cis9 | 0.00 | 0.00% |
| C16:1 cis11 | 0.00 | 0.00% |
| C16:2 cis7, 10 | 0.22 | 0.54% |
| C16:2 cis9, 12 | 0.00 | 0.00% |
| C16:3 cis4, 7, 10 | 0.00 | 0.00% |
| C17:0 | 0.19 | 0.48% |
| C16:3 cis6, 9, 12 | 0.00 | 0.00% |
| C16:3 cis7, 10, 13 | 0.00 | 0.00% |
| C17:1 cis10 | 0.00 | 0.00% |
| C16:4 cis4, 7, 10, 13 | 0.00 | 0.00% |
| C16:4 cis6, 9, 12, 15 | 0.00 | 0.00% |
| C18:0 (SA) | 13.48 | 33.76% |
| C18:1 cis6 + 7 + 8 + 9 | 2.49 | 6.23% |
| C18:1 cis11 | 0.00 | 0.00% |
| C18:1 cis12 + C18:2 cis5, 9 | 0.00 | 0.00% |
| C18:2 cis6, 9 | 0.00 | 0.00% |
| C18:2 cis9, 12 | 2.57 | 6.44% |
| C18:2 trans9, 12 | 0.00 | 0.00% |
| C18:3 cis6, 9, 12 | 3.93 | 9.84% |
| C19:0 | 0.00 | 0.00% |
| C18:3 cis8, 11, 14 | 0.00 | 0.00% |
| C18:3 cis9, 12, 15 | 0.00 | 0.00% |
| C18:4 cis6, 9, 12, 15 | 0.00 | 0.00% |
| C18:2 cis9, 11 | 0.00 | 0.00% |
| C20:0 | 0.39 | 0.98% |
| C20:1 cis11 | 0.00 | 0.00% |
| C20:2 cis11, 14 | 0.00 | 0.00% |
| C20:3 cis8, 11, 14 | 1.04 | 2.61% |
| C21:0 | 0.00 | 0.00% |
| C20:4 cis5, 8, 11, 14 (ARA) | 1.29 | 3.23% |
| C20:3 cis11, 14, 17 | 0.00 | 0.00% |
| C20:4 cis8, 11, 14, 17 | 0.00 | 0.00% |
| C20:5 cis5, 8, 11, 14, 17 (EPA) | 4.48 | 11.21% |
| C22:0 | 0.20 | 0.51% |
| C22:1 cis13 | 0.00 | 0.00% |
| C22:2 cis13, 16 | 0.00 | 0.00% |
| C22:4 cis7, 10, 13, 16 | 0.00 | 0.00% |
| C22:3 cis13, 16, 19 | 0.00 | 0.00% |
| C22:5 cis4, 7, 10, 13, 16 | 0.00 | 0.00% |
| C22:5 cis7, 10, 13, 16, 19 | 0.00 | 0.00% |
| C24:0 | 0.00 | 0.00% |
| C22:6 (DHA) | 0.00 | 0.00% |
| C24:1 | 0.00 | 0.00% |
| Total FAME | 39.93 | 100.00% |

TABLE 14
List of Sequences and their demonstrated Functions
Sequences with an asterisk were codon optimized
for expression in Aurantiochytrium

| SEQ ID NO: | Function | Source |
|---|---|---|
| 1 | ω3-desaturase | Labyrinthulomycete |
| 2 | Δ5-desaturase | Labyrinthulomycete |
| 3 | Δ5-desaturase | Labyrinthulomycete |
| 4 | Δ5-desaturase | Labyrinthulomycete |
| 5 | Δ9/Δ6 elo | Labyrinthulomycete |
| 6 | Δ6/Δ9 elo | Labyrinthulomycete |
| 7 | Δ6/Δ9 elo | Labyrinthulomycete |
| 8 | Δ6/Δ5/Δ9 elo | Labyrinthulomycete |
| 9 | Δ6/Δ8 desat | Labyrinthulomycete |
| 10 | Δ6-desaturase | Labyrinthulomycete |
| 11 | Δ6-desaturase | Labyrinthulomycete |
| 12 | Δ6-desaturase | Labyrinthulomycete |
| 13 | Δ12-desaturase | Labyrinthulomycete |
| 14 | Δ9-desaturase | Labyrinthulomycete |
| 15 | Δ9-desaturase | Arxula |
| 16 | C16 elo | Arxula |
| 17 | C16 elo | Labyrinthulomycete |
| 18 | Δ5 elo | Labyrinthulomycete |
| 19 | Δ5 elo | Labyrinthulomycete |
| 20 | Δ4 desat | Labyrinthulomycete |
| 21 | ω3-desat | Labyrinthulomycete |
| 22 | ω3-desat | Labyrinthulomycete |
| 23 | ω3-desat | Pavlova |
| 24 | Δ 12-desat | Labyrinthulomycete |
| 25 | Δ 12-desat | Nannochloropsis |
| 26 | Δ12-desat | Isochrysis |

Although the invention has been described with reference to the above examples, it will be understood that modifications and variations are encompassed within the spirit and scope of the invention. Accordingly, the invention is limited only by the following claims.

What is claimed is:

1. A recombinant Labyrinthulomycetes cell producing a FAME profile comprising:
a. greater than 12% ARA; or
b. greater than 8% EPA; or
c. greater than 20% SA; or
d. greater than 10% OA; and
e. less than 10% DHA.

2. The recombinant cell of claim 1 wherein the cell has a FAME profile having less than 5% DHA.

3. The recombinant cell of claim 1 wherein the cell is viable on a medium that is not supplemented with a PUFA.

4. The recombinant cell of claim 1 wherein the recombinant cell produces a FAME profile having greater than 12% ARA.

5. The recombinant cell of claim 4 wherein the cell has a FAME profile having less than 5% DHA.

6. The recombinant cell of claim 5 wherein the cell is viable on a medium that is not supplemented with a PUFA.

7. The recombinant cell of claim 1 wherein the recombinant cell produces a FAME profile having greater than 8% EPA.

8. The recombinant cell of claim 7 wherein the cell produces a FAME profile having less than 5% DHA.

9. The recombinant cell of claim 8 wherein the cell is viable on a medium that is not supplemented with a PUFA.

10. The recombinant cell of claim 1 wherein the recombinant cell produces a FAME profile having greater than 25% SA.

11. The recombinant cell of claim 10 wherein the cell produces a FAME profile having less than 5% DHA.

12. The recombinant cell of claim 11 wherein the cell is viable on a medium that is not supplemented with a PUFA.

13. The recombinant cell of claim 1 wherein the recombinant cell produces a FAME profile having greater than 10% OA.

14. The recombinant cell of claim 13 wherein the cell produces a FAME profile having less than 5% DHA.

15. The recombinant cell of claim 14 wherein the cell is viable on a medium that is not supplemented with a PUFA.

16. A biomass produced by a recombinant Labyrinthulomycetes cell of claim 1 and having a FAME profile comprising a parameter selected from the group consisting of: greater than 8% EPA, greater than 12% ARA, greater than 12% OA, greater than 15% PA, and wherein the parameter is produced by an exogenous pathway.

17. The biomass of claim 16 wherein the biomass has a FAME profile of greater than 10% EPA.

18. The biomass of claim 16 wherein the biomass has a FAME profile of greater than 12% ARA.

19. The biomass of claim 17 wherein the biomass has a FAME profile having less than 10% DHA.

20. A food product or ingredient comprising the biomass of claim 16.

21. A food product or ingredient comprising the biomass of claim 19.

22. A microbial oil comprising at least one polyunsaturated fatty acid synthesized by a Labyrinthulomycetes cell, wherein the oil has a FAME profile having a content of EPA that is higher than the content of DHA.

23. The microbial oil of claim 22 wherein the cell is a member of a genus selected from the group consisting of: Aurantiochytrium, a Schizochytrium, a Thraustochytrium, and an Oblongichytrium.

24. The microbial oil of claim 23 wherein the FAME profile is greater than 10% EPA.

25. The microbial oil of claim 24 wherein the oil has a FAME profile of less than 5% DHA.

26. The microbial oil of claim 23 wherein the oil has a FAME profile having greater than 10% EPA and less than 1% DHA.

27. The microbial oil of claim 22 wherein the microbial oil is an extracted and unconcentrated oil.

28. A microbial oil comprising at least one polyunsaturated fatty acid synthesized by a Labyrinthulomycetes cell, wherein the oil has a FAME profile having a content of ARA of greater than 15%.

29. The microbial oil of claim 28 wherein the cell is a member of a genus selected from the group consisting of: Aurantiochytrium, a Schizochytrium, a Thraustochytrium, and an Oblongichytrium.

30. The microbial oil of claim 29 having a FAME profile with less than 5% DHA.

31. A food product or food ingredient comprising the microbial oil of claim 22.

32. The food product of claim 31 wherein the food product is animal feed.


SEQUENCE LISTING (54 sequences)

SEQ ID NO: 1 - protein, 432 aa, Oblongichytrium sp.
  misc_feature: SG1EUKT1902255_(omega-3_desaturase)
MCRAQVAAEATVGEPMAEPSSNEPANVSKLAPEDRWVETLDLDGFKEEIQALGKELAANQGEDDVRHLNKILWWNTILTVLGIGTMWASPNLFTIICLSVATFSRWTMIGHHTCHGGYDKCEPSGRFNRFKFAVGSLYRRAVDWFDWMLPEAWNIEHNQLHHYHLNEAKDPDLVQENLSYLRDLNIPVALKYVVVAFFMGTWKWFYYAPNTFHQLEIARLRREGGEVSKLERVHVTVASLAEAPEYYNVTKLFTHVLGPYFVYRFVLMPLPILVLMGPTYFYNAIINLVLADILTNIHGFIAIVTNHAGNDLYTFEKHCKPRGAHFYLRQVISSVDFACGNDLVDFLHGWLNYQIEHHLWPDLSMLSYQRSQPRVKAICAKYGVPFVQESVWIRLKKTIDIMVGNSTMRPFPIEFEPNDYDDVATNGTKKEN

SEQ ID NO: 2 - protein, 439 aa, Thraustochytrium sp.
  misc_feature: SG1EUKT699409_(delta-5_desaturase)
MGRGGEGEAPKRAELHGATSTGRKVVLIEGQMYDVTNFRHPGGSIIKFLSTDGSEVVDATEAYREFHCRSSTADKYLKALPKVDGPIKMKFDAKEQARRDAITRDYAILREQLVKEGFFKPVPLHVLYRCVEILAMFALSFYLFSFKNNMLAIAAAVLVGGIVQGRCGWLMHEAGHYSMTGYIPLDLRLQELIYGVGCGMSGAWWRNQHNKHHATPQKLKHDVDLDTLPLVAFNEKIAARVKPGSFQAKWLSAQAYIFAPISCLLVGLFWTLFLHPRHMIRTKRRAEFVWIVTRYLGWFGLLHSFGYSFGDAFKLYLVTFGVGCTYIFTNFAVSHTHLPVTNPDEFLHWVEYAALHTTNVSNDSWFVTWWMSYLNFQIEHHLFPCCPQLHHPKIAPRVRQLFEKHGMVYDERPYVQALKDTFNNLHSVGNAVNSSKKTA

SEQ ID NO: 3 - protein, 435 aa, Thraustochytrium striatum
  misc_feature: SG2EUKT444939_(delta-5_desaturase)
MGRGGQKEAEKAGLVGAKQRKTILIEGQVYDVTNFRHPGGSIIKFLTTDGSAHIDATNAFREFHCRSGSAHKYLKSLPKVDGPVKMMYDDKEQARRDAMTKDYAEFRAQLVAEGKFDPSPMHATYRVVELVSLFVASFYLFSLGSPLAVVAGVLVSGIAQGRSGWLMHEAGHYSLTGSIPIDLRIQEIVYGLGCGMSGAWWRNQHNKHHATPQKLKHDVDLDTLPLVAFNEKIAAKVRPGSFQAKWLSMQAYIFAPVSCLLVGLFWTLFLHPRHILRTSRGFEAVCLATRYAGWFALMSSMGFAPLDSLKLYLASFGLGCVYIFTNFAVSHTHLDVTDPDEYRHWVEYGALHTTNVSNDSYLVTWWMSYLNFQIEHHLFPSMPQFRHPTIAPRVRELFAKHGLEYDERSYVQAMRDTFGNLNAVGKAAGQAPKAA

SEQ ID NO: 4 - protein, 439 aa, Thraustochytrium sp.
  misc_feature: SG1EUKT2058746_(delta-5_desaturase)
MGKGSEGRSAVNGVQTSANSQGNKPKTILIEGVLYDVTNFRHPGGSIIDFLTEGEAGVDATQAYREFHQRSGKADKYLKSLPKLDVSKVKSRFSSKEQARRDAMTKDYAEFREQLIKEGYFDPSLPHMTYRVVEIVVLFVLSFWLMGQSSPLALALGIVVSGISQGRCGWVMHEMGHGSFTGVIWLDDRLCEFFYGVGCGMSGHYWKNQHSKHHAAPNRLEHDVDLNTLPLVAFNERVVRKVRPGTLLALWLRVQAYLFAPVSCLLIGLGWTLYLHPRYMMRTKRWMEFVWIAVRYVAWFGVMGALGYTPGQSLGMYLCAFGLGCIYIFLQFAVSHTHLPVSNPEDQLHWLEYAADHTVNISTKSWFVTWWMSNLNFQIEHHLFPTAPQFRFKEISPRVEALFKRHGLPYYDMPYTSAVSTTFANLYSVGHSVGDAKRD

SEQ ID NO: 5 - protein, 272 aa, Thraustochytriidae sp.
  misc_feature: SG1EUKT2025971_(delta9/delta6_elongase)
MEDLERYKGMAESLAKYATSAAFKWQVTYSKEDSYVGPMMISEPLGLLVGSTALYFVTLAVTYMLRGYLGGLMALRGAHNLGLCLFSGAVWIYTTYLMVQDDHFASLESATCKRLTHPHFQLISFLFAASKVWEWFDTVLLIIKGNKLRFLHVLHHATTFWLYAIDHIFLSSIKYGVAVNAFIHTVMYAHYFRPFPKQFRPLITQLQIVQFIFSIAIHTAIYFHYDCEPLVHTHFYEYLTPYYIVVPFLFLFLNFYVQQYILAPSKPKTKSA

SEQ ID NO: 6 - protein, 272 aa, Thraustochytrium sp.
  misc_feature: SG1EUKT708906_(delta-6/delta-9_elongase)
MNSSVWDGVVAKAQGTVDAWMGEVPEYEHTKGLPMMDISTMLAFEVGYVSMLVFGIPFMKNQEKPFQLKTFKLFHNFFLFALSLYMCLETVRQAVLGGYSVFGNDLETGDAPHVTGMSRIVYIFYVSKAYEFVDTAIMILCKKFNQVSFLHVYHHATIFAIWWAIAKFAPGGDAYFSVILNSFVHTVMYAYYFFSAQGYTFVKPIKPYITSMQMTQFMAMLVQSLYDYLYPCEYPQALVRLLGVYMITLLALFGNFFVQSYLRKPAPKAKSA

SEQ ID NO: 7 - protein, 276 aa, Thraustochytrium sp.
  misc_feature: SG1EUKT678360_(delta6/delta9_elongase)
MSTMMNGTSAWDQLVQATDTSISEFMGEDVKPYPLTDGIFTRVETLIICELFYFALIGLGVPIMKAQEKGFELKGYKLFHNLFLLTLSGYMAIETIRQAYLGGYKLFGNDMEKGNEPHAEGMARIVWIFSVSKVYEFMDTAIMILGKRFRQVSFLHCYHHMSIFAIWWAIAKYAPGGDAYFSVILNSTVHFVMYSYYGFTALGFNFVRKIKPYITTMQLTQFMSMLIQSLYDYMYPCDYPQSLVRLLGVYMLTLIALFGNFFVQNYMKKPQKKKTA

SEQ ID NO: 8 - protein, 277 aa, Thraustochytriidae sp.BURABG 162
  misc_feature: SG1EUKT1962668_(delta6/delta5/delta9_elongase)
MDVAMEQWKRFVETVDKGIVDFMEGEKTNEMNAGKPLISTEEMMALIVGYLAFVVFGSGFMKVFVEKPFELKYLKLAHNIFLTGLSMYMATECARQAYLGGYKLFGNPMEKGNEAHAPGMANIIYIFYVSKFLEFLDTVFMILGKKWKQLSFLHVYHHASISFIWGIIARFAPGGDAYFSTILNSCVHVMLYGYYASTTLGYGFMRPLRPYITTIQLTQFMAMVVQSVYDFYNPCDYPQPLVKLLFWYMLTMLGLFGNFFVQQYLKPKPATKKQKTI

SEQ ID NO: 9 - protein, 457 aa, Botryochytrium radiatum
  misc_feature: SG1EUKT2107789_(delta6/delta8_desaturase)
MGRGGQKTEGLSAQPQQEREQLLKGKWESVVRIDGVEYDVTDYMRKHPGGSVIKYGLANTGADATHLFNAFHMRSKKAKMVLKSLPKRQPQLEIQPGQLPEEQTKEAEMLRDFEKLENELRAEGYFEPSFWHRLYRFTELAVMFSLGLYCFSLRTPLSIAAGVFLHGLFGAFCGWAQHEGGHGSLYHSLWWGKRAQAMMIGFGLGTSGDMWNMMHNKHHAATQKVNHDLDIDTTPLVAFFNTAFEKNRYRGFAKWWVRFQALTFLPITSGCFVMWFWLLFLHPRRVVQKGNVEEGFWMLSSHIVRTYLFQLCTGWESLAACYLVGYWGAMWVSGVYLFGHFSLSHTHLDIVDADVHKNWVRYAVDHTVDISPQNPLVSWIMGYLNLQVLHHLWPQMPQYHQPAVSKRVAAFCKKHGLNYRVVSYFEAWKLMFSNLSNVSDHYMKNGFERPAKKTKAQ

SEQ ID NO: 10 - protein, 458 aa, Schizochytrium sp.
  misc_feature: SG1EUKT1948090_(delta6_desaturase)
MGRGGQKVESGVQQGPESELLSKPAGSWEQVVQIDGVEYDVTNFMRKHPGGKVLRYGLANSGADATQLFKAFHMRSKKAHLILKSLPKRQPQLKIQPGQLPTEESKEGEMLRDFVKFEKELEEEGFFEPSFAHRVYRLGELAVLFALGLYLFTLRTPLAIAAGVAVHGLFGARCGWVQHEAGHGSFMRSIWWGKRVQAMCIGFGLGTSGDMWNMMHNKHHAATQKVGHDLDLDTTPLVAFFNTAFEKNPKRGFSKWWTRFQALTFVPITSGCFVMWFWLLFLHPRRVIQRGKVDEGLWMLSSHIVRTALFKTLAGFESWAAAYAVGYWGAMWVSGMYLFGHFSLSHTHLDIVEEDVHKNWVRYAVDHTVDISPDSWLVNWTMGYLNLQNIHHLFPTMPQFRQPEVSRRFAVFCKKHGLNYRVVSYWHAWYLMFNNLLTVGAHYADNGLNRDLVKAKAA

SEQ ID NO: 11 - protein, 456 aa, Schizochytrium sp.
  misc_feature: SG1EUKT1988755_(delta6_desaturase)
MGRGGQKSEAVAASATTPLKTADGAKPRSWEKVVLIDGVEYDVTNFIKRHPGGSVIKYALAEEGADATAIYNAFHIRSRKADLMLKSLPSRKPQLEVQPGQLVDEDSKEGEMLRDFAKFEQQLKDEGFFEPSNLHVLYRVTELAAIFALGLYLFSLRTPLAILGGVIAHGLFGGRCGWVQHEGGHGSLFTSLWLGKRVQACLIGFGLGTSGDMWNMMHNKHHAATQKVNHDLDIDTTPLVAFFNTAFEKSRFSSAFNKFWIRFQAFTFLPVTSGVFVMLFWLLFLHPRRVIQRGLPEEGFWMITSHIVRTALFKAATGWSSWAACYAVGYWGSMWVSGMYLFGHFSLSHTHLDVVEQDVHKNWVRYAVDHTVDISPGNPLVCWMMGYLNLQTIHHLFPVMPQYKQVEVSRRFAVFCDKHGLNYRRSTYFGAWYDMFNNLWTVGQHYHANGVQKKLN

SEQ ID NO: 12 - protein, 457 aa, Thraustochytrium sp.
  misc_feature: SG1EUKT677770_(delta6_desaturase)
MGRGGQNTSTSNLVASKTMNGKETQTETQWVRINNVEYDITNFVKRHPGGNVINYGLANTGADATQLFNAFHMRSEKAQKMLKSLPQREPQCELQPGQLPEGDDKEAEMLRAFEKFEKQLEAEGFFKPSLAHDIYRIVELAGLFLLGLYFFSFKTPLMIAAGVVTHGLFGGRCGWVQHEAGHGSFTENLFLGKRIQAFFIGFGLGASGSLWNKMHNKHHAATQKVGHDMDLDTTPMVAFFKDAFEKNRFRGFSKAWIQFQAFTFLPVVSGCFIMLFWLTFLHPMHVIKGGFVDQGFWMLSSHIIRAALFKLCTGWQSWAACYAVGYWGSMWVSGMYLFGHFSLSHTHLDVIDADQHKNWVRYAVDHTVNISPGNPFVDWIMGYLNCQIEHHLWPAMPQFRQPQVSKRLEAFCIKYGLEYRKMSYPQAWYAMFSNLHNVGHHYHEHGLNEELRSKKKQ

SEQ ID NO: 13 - protein, 405 aa, Thraustochytrium sp.
  misc_feature: SG1EUKT710829_(delta12_desaturase)
MCKNEAQSKSAALRGAPPQQQDQPLPSIKDIRAAVPAHCFQRSALRSSLFVVRDAALAALVGWAAYKTLPTDLGNPLALAGWLAYALVQGTVLTGLWVIGHECGHQAFSESALVNDSFGFVIHSALLVPYFSWARTHAVHHARCNHLLDGETHVPDLKRKVHGMYAKILDVVGEDAFVLLQIVLHLLFGWIMYLVMHATGSRRSPVTKERYKRKPNHFVPLASNELFPAKLRFKVLLSTVGVLGMIGALCYAGSLYGGKIVSLLYVGPYLVVNAWLVTYTWLQHTDPEVPHYGEAEWTWLKGALSTIDRPYPWIVDELHHHIGTTHVTHHVFHELPHYHAQEATAALKAVLGPHYRYDPTPIVKAMWKTAETCHYVEDVKGVQYLRSIVTERRQAAQAAAKAKAL

SEQ ID NO: 14 - protein, 386 aa, Ulkenia sp.
  misc_feature: SG1EUKT1945754_(delta9_desaturase)
MTHNSSFAGDALHADASVNRVAGLAGTVVFGTVLAYLGTRQPKESHSMKLRDMPEGYKAQNDLEQKAIDAYERRQELSTIPWLFQNIRWVMSIYIGGIHLLAIYALPHLLDCKWETLLGMLAFYVLGGFGITGGAHRLWSHRTYKANAVFRFVVMICNSIANQGTIYHWSRDHRTHHKYSETKADPHNALRGFFFAHVGWLFFKKDPRVKFAGNHIPMNDLAALPEVQLQKRLDPWWNLFWCFGAPTLCGHFLWGETVLKSFLLMGVLRYALCLNGTWLVNSAAHLYGGHPYEDINPAENPVVAFFSMGEGWHNWHHAFPHDYSASELGVSSQFNPTRLVIDFCALFGIVWDRKTATDHWETRKKKKGYKHVDLQGAPFFRQRVVS

SEQ ID NO: 15 - protein, 486 aa, Arxula adeninivorans
  misc_feature: SG2EUKT459643_(delta9_desaturase)
MNGPEEVNLEEVQAIASGAEVRAKVNINRRRQEEQAAAAAASSGSTKTHISEQAFTLANWHKHFNWINTTIIAIIPAIGFLSVPFIPVHGKTLAWAFVYYFLTGLGITAGYHRLWAHRAYSASWPLRVFLALLGAGAGEGSVKWWSNGHRTHHRYTDTDKDPYNAKRGFWFSHMGWMMFKQNPKLKGRCDISDLICDPIIRWQHRHYIWIMAAMSFVFPSVVAGLGWGDYLGGFVFAGILRQFVVHQSTFCVNSLAHWLGEQPFDDNRSPRDHVLTAFATLGEGYHNFHHEFPSDYRNAIKWYQYDPTKIFIWTMKQLGLASNLQTFSQNAIEQGLVQQKQKKLDRWRARLNWGVPIEQLPVIEYDDFKDESSSRSLVLISGIVHDVTDFIDKHPGGKALIKSAIGKDGTAVFNGGVYKHSNAAHNLLATMRVAVIRGGMEVEVWKRAQGEKKDVDPVADSAGDRILRAGDQPSRVPEARVSGRAA

SEQ ID NO: 16 - protein, 334 aa, Arxula adeninivorans
  misc_feature: SG2EUKT457191_(C16_elongase)
MLEVTFPPTLDRPFGVYLYGLFDALTNGWATRFQFAQDSGIPFSSRWEVAAGIVTYYVVIFGGREVLKNAPVIRLNFVFQIHNLILTLLSLGLLLLLVEQLIPIIVRHGVLYAICNSGSWTQPIVTVYYLNYLTKYYELFDTVFLVLRKKPLTFLHTYHHGATALLCFTQLIGHTSVSWVPIVLNLFVHVIMYYYYFLSALGVRNIWWKEWVTRTQIIQFVVDLVFVYFATYTYFTNKYWPWLPNKGTCAGEEFAAIYGCALLTSYLFLFIAFYIRVYTKAKAKGRKRAASAAAKATTGVVTADRPSTPIATTNGAATGAAGATGSVKSRSRKA

SEQ ID NO: 17 - protein, 378 aa, Oblongichytrium sp.
  misc_feature: SG1EUKT1905730_(C16_elongase)
MATSMNDSLPEGATAMNTLQRLLSFQNEFHSKEVLTWHRDHAEIPIICLSLYLVMVFAGPDLMKHREPFKLKRTFAAWNFFLSVFSILGAYHMVPLLLGRLWEHGFKATVCSHPEWYFNGPSGFWLCLFIYSKFAELFDTAFLVLRKRNVIFLHWFHHATVLLYCWHAYHHAIGAGVWFACMNYCVHSVMYFYYFMTNVGLYRVVAPFAQAITTVQILQMVGGMAVLLQVAHARLTEDPTACEVDSANWKLGLAMYASYFVLFVLLFVKKYLSPAPSSAQSKRTSGNDTSSATGSTTKSNSSSTKGDADASAGARRRARVSVTCPPGLDDVPVFGDSASVSKLDASGFFHSQQTVEEARRAQDDADRAQAMRNKKKAL

SEQ ID NO: 18 - protein, 313 aa, Oblongichytrium sp.
  misc_feature: SG1EUKT663149_(delta5_elongase)
MSISEQAAAGAPTKPLPCKDAVDSANAAARILKKEESVSSFQWQKALCIVAMELYLMKMIFDESKNKVNPFGEASWVYPLVGTVCYLLFVFLGKKYMATREAFNVKQYMIMYNLYQTVFNVYVVVCFFQEIYRRKLPAAGTKFVPGPEEFNLGFLIYAHYQNKYLELLDTVFMVVRKKNNQISFLHVYHHTLLIWSWYAVLKINPGGDAYFGALANSIIHVVMYSYYLLALLGVPCPWKKYVTIMQLVQFMVVFSQGLYCLYLGSTPTMLILLQQFVMINMLVLFGNFYMKSYRKKAADRKAKAEEENTKKTE

SEQ ID NO: 19 - protein, 312 aa, Oblongichytrium sp.
  misc_feature: SG1EUKT1992701_(delta5_elongase)
MAARVDAKGVQAKTQVAKTVPRSRKVDRSDGFFRTFNLCALYCSAFAYAYKNGPTDNDENGLFFSKSPFYAFLVSDAMTFGAPLAYVVAVMLLSRYMADKKPMTGFIKTYVQPVYNVVQIVVCGWMAWGLLPQVTLTNPFGLNTQRDPQIEFFVMVHLLTKFLDWSDTFMMILKKNYAQVSFLQVFHHATIGMVWSFLLQRGWGSGTAAYGAFINSVTHVIMYTHYFVTSLNINNPFKRYITAFQLSQFASCIVHAVLALLFEEVYPIEYAYLQISYHMIMLYLFGCRMDWSPLWCTGEVDGLDEANKKKTN

SEQ ID NO: 20 - protein, 510 aa, Thraustochytrium sp.
  misc_feature: SG1EUKT698636_(delta4_desaturase)
MGEEKKITLAQVREHNLPTDAWCVIHDKVYDVTNFAKIHPGGDLVLLAAGKDATILYETYHIRGVPDAVLAKYRIGTLEAPEVKSTSGLDSASYYSWDSEFYKVLKKRVVARLEKLKLERRGGIEIWTKAFMLMTGFWGSLYLMCTLNPDGWAIPAAMSVGVFAAFVGTCIQHDGNHGAFAKSKFLNKAAGWTLDMIGASAMTWEMQHVLGHHPYTNLIEMENGVQKVSGKPVDTKKVDQESDPDVFSTYPMLRLHPWHRKRFYHKFQHIYAPFIFGFMTINKVITQDLGVLLNKRLFQIDANCRYASPGYVFRFWFMKFLTMLYMVGLPMYMQGPLQGLKLYFVAHFTCGELLATMFIVNHIIEGVSYASKDAVKGGMAPPRTVHGVTPMNETQQALEKKEKLAPTKKIPLNDWAAVQCQTSVNWAIGSWFWNHFSGGLNHQIEHHLFPGLTHTTYVYIHDVVKDTCAEYGVPYQHEESLYSAYWKMLSHLKTLGNEPMPAWEKDHPKA

SEQ ID NO: 21 - protein, 436 aa, Oblongichytrium sp.
  misc_feature: SG1EUKT2148609_(omega3_desaturase)
MCNPKSLSAPSGVEAHVVQEHVPVGEHDKWLETLDFDAFKEDMQRLGKSLADGQGQQDVDHLQKFVWWNRIVTLCGILTMACFPNPFTVFCLSLGTFSRWTIIAHHVCHGGFDKADKSKRYNRFTFGVGSLYRRMVDWFDWMLPEAWNVEHNQMHHYHLNEAKDPDLVELNLSPLRENDAVPMPVKYMVVFFFMCTWKWFYYAPSTFIQLAQARLRRQGVSTEGMPRAHLTIASIFEAPDFVSIKELFLRVLFPYFAYRFLLVPLPVLALWGPKLFSYAIINLVLADIVTNIHSFIAVVTNHAGDDLYVFEKHCKPRGAHFYLRQVISSVDFTCGNDLMDFMHGFLNYQIEHHLWPDLSMLSYQRAQPLVKQLCAKHGVPYVQESVWIRLKKTIDIMVGKTSHRKFPSQYEPNDYDHLKTAAATTTAMEEPEAEAK

SEQ ID NO: 22 - protein, 432 aa, Oblongichytrium sp.
  misc_feature: SG1EUKT664904_(omega3_desaturase)
MCRAQVAAEATVGEPMAEPSSNEPANVSKLAPEDRWVETLDLDGFKEEIQALGKELAANQGEDDVRHLNKILWWNTILTVLGIGTMWASPNLFTIICLSVATFSRWTMIGHHTCHGGYDKCEPSGRFNRFKFAVGSLYRRAVDWFDWMLPEAWNIEHNQLHHYHLNEAKDPDLVQENLSYLRDLNIPVALKYVVVAFFMGTWKWFYYAPNTFHQLEIARLRREGGEVSKLERVHVTVASLAEAPEYYNVTKLFTHVLGPYFVYRFVLMPLPILVLLGPTYFYNAIINLVLADILTNIHGFIAIVTNHAGDDLYTFEKHCKPRGAHFYLRQVISSVDFACGNDLVDFLHGWLNYQIEHHLWPDLSMLSYQRSQPRVKAICAKYGVPFVQESVWIRLKKTIDIMVGNSTMRPFPIEFEPNDYDDVATNGTKKEN

SEQ ID NO: 23 - protein, 363 aa, Pavlova pinguis
  misc_feature: SG1EUKT1024722_(omega3_desaturase)
MCPPATHDATPIKDGANRAEIVAESKLTLQDIRKAIPQECFEKNTARSMLYLVRDLAICATAPLVYPYVAASGNPLAYLAYWNFYGFFMWCLFVVGHDCGHTTFSPNKTLNDICGHIAHAPLMVPYYPWAMSHRRHHMYHNHQKKDASHPWFSKSSLKKLPAFTRNFLKSPLAPFLAYPIYLFEGSFDGSHVFPLSKLYKGSQMRARVECAISAVTVFAFGTAAYMFCGDARTLALAYGGCYACFSFWLFMVTYLQHHDHGTLVYDDSDWTYLKGALETVDRKYGFGLDNLHHNISDGHVVHHLFFTQVPHYHLTKATEQVAPLLRKAGVYKRVDHDNFLKDFWRTFFTCNFTGWKWANGKDN

SEQ ID NO: 24 - protein, 397 aa, Oblongichytrium sp.
  misc_feature: SG1EUKT666457_(delta12_desaturase)
MANLSPMDQERLSEITPFKPTVDHPVPTIKQVRDAIPAECFERKLWKGLALVVRDGIIIAGLGFAAWQLPFANLTPAMMAVWLVYAVLQGTALTGWWVLAHECGHGAFSSYSWINDTIGFVLHTVLLVPYFSWQYSHGKHHAKTNHLLDGETHVPPKGVRGLNAKLADLLGEDAFAMWQLVSHLVFGWPLYLIMNATGARRRFDRVRYSSSPSHFSPTSELFPSRWRPWVALSSVGIIAWLTVLYVVSTHIGTQRLALMYGFPYLVVNGWLVLYTWLQHVNEYVPQYGEDEWTWMKGALATVDRPYWGPVDWMHHHIGTTHVAHHVFSHMPCYNAPRATIFLKRFLGDLYHYDERPISKAAWQVAKECHYVDDLQGRQLMKSIFKHHKKEASKKRNQ

SEQ ID NO: 25 - protein, 438 aa, Nannochloropsis oceanica
  misc_feature: EMRE1EUKT5767207_(delta12_desaturase)
MGRGGEKTVTPLRKKTLLDAASTISGTVRPSKAVEALPTEELRKKAAQYGINTSVDRETLLRELAPYGDILLRNDAPKSLPLAPPPFTLSDIKNAVPRHCFERSLSTSLFHLTIDLIQVAVLGYLASLLGHSDVPPMSRYILWPLYWYAQGSVLTGVWVIAHECGHQSFSPYESVNNFFGWLLHSALLVPYHSWRISHGKHHNNTGSCENDEVFAPPIKEELMDEILLHSPLANLVQIIIMLTIGWMPGYLLLNATGPRKYKGLSNSHFNPNSALFSPKDRLDIIWSDIGFFVALACVVYACVQFGFQTVGKYYLLPYMVVNYHLVLITYLQHTDVFIPHFRGSEWTWFRGALCTVDRSFGWLLDHTFHHISDTHVCHHIFSKMPFYHAQEASEHIRKALGDYYLKDDTPIWKALWRSYTLCKYVDSEETTVFYKQRA

SEQ ID NO: 26 - protein, 386 aa, Isochrysis sp.
  misc_feature: SG1EUKT1012028_(delta12_desaturase)
MGKGGSAGKSSAVERLGPLAATIPEPSKEITKGSIRAAIPPHLFQRSYVHSLGHLVMDLLWVAATWLLALQAASVLPSGFAPLVWAAYWFYQGINLTALWVLAHECGHGGFTDSRLVNDIVGFVLHSALLTPYFSWAITHAKHHHYTNHMTNGETWVPSTANPEKASVKFAKSPIGTIRRIVVVALLGWYTYLFTNATGAKQNAGQSHFSPSSRALFKAKDANLVRASNLGMIACLAVLAKCVSVWGFSAVFFNYLVPQTICNFYLCAITFMQHTHESVPHFNSEEWTWLRGALSTIDRSMGSHVDWRLHHIVDSHVVHHIFSDMPFYGAKAATPYVKEHLGIYYKSNLGSKVLGSEYLGYWKDFYSSMSRAVVVGVGEDNFMWFR

SEQ ID NO: 27 - protein, 1299 aa, Oblongichytrium sp.
  misc_feature: SG1EUKS305451_(omega3_desaturase)
ATGTGCCGAGCACAGGTCGCTGCTGAAGCGACGGTTGGCGAGCCAATGGCCGAACCATCGTCGAATGAGCCTGCCAATGTCAGCAAGTTGGCGCCGGAAGACCGCTGGGTTGAAACCCTTGACTTGGATGGCTTTAAGGAAGAAATCCAGGCGCTTGGAAAGGAGCTCGCTGCAAACCAGGGCGAAGATGATGTTCGCCACCTGAACAAGATTCTTTGGTGGAACACCATCCTCACCGTGCTTGGGATCGGAACGATGTGGGCCTCCCCGAATTTGTTTACCATCATCTGTCTAAGTGTTGCGACATTTTCTCGCTGGACGATGATTGGCCACCACACTTGCCACGGTGGTTACGATAAGTGTGAACCATCGGGACGATTCAACCGCTTTAAGTTTGCTGTTGGTAGCCTTTATCGCCGCGCTGTAGATTGGTTTGACTGGATGTTGCCCGAGGCATGGAACATCGAGCACAACCAGCTTCACCATTACCATCTTAACGAAGCCAAGGACCCTGACCTTGTTCAGGAAAACCTCTCATATTTACGTGACTTGAACATCCCAGTAGCACTTAAGTACGTGGTGGTCGCCTTTTTCATGGGGACTTGGAAGTGGTTCTACTATGCACCAAACACATTTCACCAACTCGAAATTGCACGCCTTCGCCGTGAAGGTGGCGAAGTGAGCAAGCTTGAGCGAGTTCACGTCACTGTAGCCTCGCTTGCTGAAGCTCCTGAATATTACAACGTAACCAAGCTCTTCACCCATGTGCTTGGTCCATACTTTGTGTACCGCTTTGTACTCATGCCTCTCCCGATTTTGGTCTTGATGGGCCCCACTTATTTCTACAATGCCATCATCAACCTAGTGCTCGCTGATATTTTGACAAACATTCATGGCTTTATTGCCATTGTCACGAATCACGCAGGGAACGACCTGTACACTTTTGAAAAGCACTGCAAGCCACGTGGTGCGCACTTTTACTTGCGACAAGTGATTAGCTCTGTAGACTTCGCTTGCGGCAACGATCTCGTCGATTTTCTTCACGGATGGCTCAACTACCAGATTGAGCACCATCTCTGGCCAGATCTCTCTATGTTGAGCTACCAGCGATCGCAGCCACGTGTAAAGGCCATCTGTGCCAAGTACGGCGTTCCATTTGTTCAGGAAAGCGTCTGGATTCGTCTCAAGAAGACCATCGATATTATGGTTGGAAACTCGACCATGCGACCATTCCCAATTGAGTTCGAACCAAACGATTACGACGACGTTGCAACCAATGGAACAAAGAAGGAAAACTAA

SEQ ID NO: 28 - DNA, 2640 bp, Thraustochytrium striatum
  misc_feature: SG1EUKS146702_(delta5_desaturase)
atgggtcgcggaggagaaggtgaagcaccaaagcgtgcagagttgcacggagcaacctccacagggaggaaagtggtcctgattgaagggcagatgtacgatgtaactaactttcgccaccctggtggatctatcatcaagttcttgtccacagatggatccgaggtggtggatgccactgaggcataccgcgagtttcactgccgatccagcactgcagacaagtacctcaaagccctgccaaaagttgatggaccaatcaaaatgaagtttgatgcaaaagaacaggcccggcgtgatgctatcacacgtgactacgcaattctccgagagcagctggtgaaagagggtttcttcaaaccagtgcctctccatgtgctctacagatgtgtcgagatcttggccatgtttgctctatctttctaccttttctccttcaagaacaacatgctggccatcgcagctgcagtccttgtgggagggattgtgcaaggaagatgtggttggcttatgcacgaggctgggcactacagtatgactggatacattccacttgacctgcgtcttcaggaactcatttatggtgttgggtgtggcatgagcggcgcatggtggagaaaccaacataacaaacatcatgcaactcctcaaaagctcaagcacgatgtggacttggacacgcttccactcgttgcgttcaatgaaaagattgcggctcgggtaaaacctggaagctttcaggccaaatggctctccgcccaggcttatatcttcgctcccatctcttgtcttcttgtgggcttgttctggaccctatttttgcaccctagacacatgatccgaactaagcgcagggctgaatttgtgtggattgtgactcggtatctcggttggtttggtttgcttcatagttttggctactcgtttggtgatgctttcaagctttacttggtcacgtttggcgttggatgcacgtacatctttaccaattttgcggtgagccacactcacttgcctgtgacgaatccagatgagtttttgcattgggtggagtacgcagcactgcacaccaccaatgtgtcgaacgacagttggtttgtcacatggtggatgagttacctcaacttccagattgagcatcacctttttccttgctgtccacagctgcatcatccgaaaattgctccacgtgttcgccaactattcgagaaacatggcatggtctatgatgaacgcccttacgtgcaagcactcaaagatactttcaacaacttgcacagtgttgggaacgcagtcaactcttcaaagaaaacagcttaaatgggtcgcggaggagaaggtgaagcaccaaagcgtgcagagttgcacggagcaacctccacagggaggaaagtggtcctgattgaagggcagatgtacgatgtaactaactttcgccaccctggtggatctatcatcaagttcttgtccacagatggatccgaggtggtggatgccactgaggcataccgcgagtttcactgccgatccagcactgcagacaagtacctcaaagccctgccaaaagttgatggaccaatcaaaatgaagtttgatgcaaaagaacaggcccggcgtgatgctatcacacgtgactacgcaattctccgagagcagctggtgaaagagggtttcttcaaaccagtgcctctccatgtgctctacagatgtgtcgagatcttggccatgtttgctctatctttctaccttttctccttcaagaacaacatgctggccatcgcagctgcagtccttgtgggagggattgtgcaaggaagatgtggttggcttatgcacgaggctgggcactacagtatgactggatacattccacttgacctgcgtcttcaggaactcatttatggtgttgggtgtggcatgagcggcgcatggtggagaaaccaacataacaaacatcatgcaactcctcaaaagctcaagcacgatgtggacttggacacgcttccactcgttgcgttcaatgaaaagattgcggctcgggtaaaacctggaagctttcaggccaaatggctctccgcccaggcttatatcttcgctcccatctcttgtcttcttgtgggcttgttctggaccctatttttgcaccctagacacatgatccgaactaagcgcagggctgaatttgtgtggattgtgactcggtatctcggttggtttggtttgcttcatagttttggctactcgtttggtgatgctttcaagctttacttggtcacgtttggcgttggatgcacgtacatctttaccaattttgcggtgagccacactcacttgcctgtgacgaatccagatgagtttttgcattgggtggagtacgcagcactgcacaccaccaatgtgtcgaacgacagttggtttgtcacatggtggatgagttacctcaacttccagattgagcatcacctttttccttgctgtccacagctgcatcatccgaaaattgctccacgtgttcgccaactattcgagaaacatggcatggtctatgatgaacgcccttacgtgcaagcactcaaagatactttcaacaacttgcacagtgttgggaacgcagtcaactcttcaaagaaaacagcttaa

SEQ ID NO: 29 - DNA, 1308 bp, Thraustochytrium striatum
  misc_feature: SG2EUKS84132_(delta5_desaturase)
atgggtcgcggagggcaaaaggaggccgagaaggccgggctggtcggcgccaagcagcgcaagaccatcctgatcgaaggccaggtctacgacgtgaccaactttaggcacccgggcgggtccatcatcaagttcctgaccacggacggctcggcgcacatcgacgccaccaacgcgttcagagagttccactgccgcagcggcagcgcgcacaagtacctcaagagcctccctaaggtggacggcccggtcaagatgatgtacgacgacaaggagcaggcgcggcgcgacgccatgaccaaggactacgccgagttccgcgcgcagctcgtggccgagggcaagttcgaccccagccccatgcacgcgacctaccgcgtggtcgagctcgtctcgctctttgtggcgtccttttacctgttctccctggggagcccgctcgccgtggtggctggcgtgctggtaagcggtatcgcccagggccgctcgggctggctcatgcacgaggccggccactacagcctcaccggcagcatcccgatcgacctccgcatccaggagatcgtctacggcttgggctgcggcatgagcggcgcctggtggcgcaaccagcacaacaagcaccatgccaccccgcaaaagctcaagcacgacgtcgacctcgacacgctgcccctcgtggccttcaacgagaagatcgcggccaaggtgcgcccgggcagcttccaggccaagtggctctccatgcaggcctacatctttgcgccggtctcgtgcctcctcgtcggcctcttctggaccctcttcctgcacccgcgccacatcctgcgcacgagccgcggctttgaggccgtctgcctggcgacgcggtacgcgggctggtttgccctcatgtcctccatgggcttcgcgccgctcgactcgctcaagctctacctggccagctttggcctcggctgcgtgtacattttcacaaactttgcggtcagccacacccacctcgacgtcaccgacccggacgagtaccgccactgggtagagtacggcgcgctgcacaccaccaacgtgtccaacgacagctacctggtcacctggtggatgagctacctcaactttcagatcgagcaccacctcttcccgagcatgccgcagttccgccacccgaccatcgcgccgcgcgtgcgcgagctcttcgccaagcacggcctcgagtacgacgagcgcagctacgtgcaggccatgcgcgacacctttggcaacctcaacgccgtcggcaaggccgcgggccaggcccccaaggctgcttag

SEQ ID NO: 30 - DNA, 1320 bp, Thraustochytriidae sp.
  misc_feature: SG1EUKS511604_(delta5_desaturase)
atgggcaagggcagcgagggccgcagcgccgtgaacggggtgcagaccagcgcgaactcgcagggcaacaagccaaagacgattttgattgaaggcgtcctgtatgatgtgacaaacttcagacacccaggtggctcgattattgacttcttgaccgagggtgaggccggcgtggacgccacgcaggcgtaccgcgagttccatcaacggtccggcaaggccgacaagtatctcaagtcccttcccaaactggatgttagcaaggtcaagtcgcggttctcgtccaaggagcaggcgcggcgagatgccatgacgaaagactatgcagaattccgcgagcaactcatcaaggagggctactttgacccctcgctcccgcacatgacgtaccgcgtggtcgagattgtcgttcttttcgtgctttccttttggctgatgggtcagtcttcaccactcgcgctcgctctcggcattgtcgtcagcggcatctctcagggtcgctgcggctgggtaatgcatgagatgggccatgggtcgttcactggtgtcatttggcttgacgaccggttgtgcgagttcttttacggcgttggttgtggcatgagcggtcattactggaagaaccagcacagcaaacaccacgcggcgccgaaccggctcgagcacgatgtagatctcaacaccttgccattggtcgccttcaacgagcgggtcgtgcgcaaggtccgaccgggcacgctgcttgcgctctggcttcgtgtccaggcatacctttttgcgccagtttcgtgccttttgattggtctcggatggacgctgtacttgcacccccggtacatgatgcgcacgaagcgctggatggagttcgtctggatcgctgtgcgctacgtggcgtggttcggcgtcatgggtgcgcttggatacacgccggggcagtccttgggcatgtacttgtgcgcctttggtctcggctgcatttacattttcctgcagttcgccgtaagtcacacccatttgcccgtgagcaacccggaggatcagctgcattggctcgagtacgcggcggaccacactgtgaacatcagcaccaagtcgtggtttgtcacgtggtggatgtcgaacctcaacttccagatcgagcaccaccttttccccacggcgccccagttccgtttcaaggagatcagcccgcgcgtcgaggccctcttcaagcgccacgggctcccttattacgacatgccctacacgagcgccgtctccaccacctttgccaacctctactccgtcggccattccgtcggcgacgccaagcgcgactag

SEQ ID NO: 31 - DNA, 819 bp, Thraustochytriidae sp.
  misc_feature: SG1EUKS512302_(delta9/delta6_elongase)
atggaggacctcgaaagatacaagggcatggcggagtcgttagccaagtacgctacgtcggcggccttcaagtggcaagtcacgtacagcaaggaggacagctatgtaggcccgatgatgatctccgaaccgctcgggctgctggttgggtcaaccgcgctgtactttgtgacactcgccgtcacgtacatgctgcgagggtatcttggcggacttatggcgctccgtggagcgcacaacctcggactgtgtctgttttctggcgccgtgtggatctatacgacctacctcatggtgcaggatgaccattttgcgagtctggaatcggcgacgtgcaaaaggctcacgcacccgcattttcagctcatcagtttcttgtttgcggcatccaaggtctgggagtggttcgacactgtattgctcatcatcaagggcaacaagttgcgttttctgcatgtcttgcaccacgcaaccaccttttggctttacgcgatcgaccacatattcctttcatccatcaagtatggtgtcgccgtgaatgcttttattcacacggtcatgtacgcgcactactttcgtcccttccccaagcagtttcgtcctctcattacgcagttgcagattgtgcagttcatctttagcattgctatccacacggcgatttactttcactatgactgcgagccgctggtgcacacgcatttttacgagtacctgacgccatattacattgtggtccccttcctctttctctttctcaacttttacgtgcagcagtacattctcgcgccgtcaaagcccaagacaaaatctgcctaa

SEQ ID NO: 32 - DNA, 819 bp, Thraustochytrium sp.
  misc_feature: SG1EUKS163594_(delta6/delta9_elongase)
atgaactcgtccgtgtgggatggtgtggtggccaaggcgcagggcaccgtggacgcctggatgggcgaggtgcccgagtacgagcacaccaagggactgccgatgatggacatcagcacgatgctggccttcgaggtgggatacgtgagcatgctggtgtttggcatcccgttcatgaagaaccaggagaagcccttccagctcaagaccttcaagctgttccacaactttttcctgttcgcgctgtcgctgtacatgtgcctcgagacggtgcgccaggctgtgctgggcggctactcggtgttcggcaacgaccttgagacgggcgacgcgccccacgtgacgggcatgtcgcggatcgtgtacatcttttacgtgtccaaggcgtacgagtttgtggacactgccatcatgatcctgtgcaagaagttcaaccaggtctccttcctgcacgtgtaccaccacgcgacgatctttgcgatctggtgggcgatcgccaagttcgcgccgggcggcgacgcgtacttctcggtgatcctcaactcgttcgtgcacacggtcatgtacgcctactactttttctctgcgcagggctacacctttgtcaagcccatcaagccgtacatcacctcgatgcagatgacgcagttcatggccatgctggtgcagtcgctctacgactacctgtacccgtgcgagtacccgcaggcgctcgtgaggctgctgggcgtgtacatgatcacgctgctggcgctctttggcaactttttcgtgcagagctacctgcgcaagccggcgcccaaggccaagtccgcgtaa

SEQ ID NO: 33 - DNA, 831 bp, Thraustochytrium sp.
  misc_feature: SG1EUKS110506_(delta6/delta9_elongase)
atgtctacgatgatgaatggcaccagtgcatgggatcaacttgtgcaagctactgacacaagcatctccgagttcatgggagaagatgtgaaaccttacccattgactgatggcatctttacgagagtggaaaccctgatcatttgcgaactgttctactttgctctcatcggactgggagttccaatcatgaaagcccaagaaaagggatttgaactaaaagggtacaagttgttccacaacttgttccttttgaccctctctggatacatggcaatcgaaaccattcgccaagcgtaccttggaggatacaaactttttggtaacgacatggaaaaaggaaacgaacctcatgctgaaggaatggctcgaattgtttggatctttagtgtcagtaaagtgtatgaattcatggacactgcaatcatgattcttggaaaacgattcagacaagttagttttcttcactgctaccatcacatgagtatttttgcaatctggtgggcaattgcaaaatatgcaccaggtggagatgcttactttagtgtcatccttaactctacggttcactttgtaatgtactcctattacggattcactgcgcttggttttaactttgtccgcaagatcaagccatatattacaaccatgcaacttactcaatttatgtccatgctcattcaatctttgtacgattacatgtacccatgtgattatcctcaaagcttggtaagacttcttggtgtatatatgcttacattgattgctttgtttggaaacttttttgttcaaaactacatgaagaaaccacaaaagaaaaaaactgcataa

SEQ ID NO: 34 - DNA, 834 bp, Thraustochytriidae sp.
  misc_feature: SG1EUKS497264_(delta6/delta5/delta9_elongase)
atggacgtggccatggagcaatggaagcggttcgtggagacggtggacaaggggattgtagactttatggaaggcgaaaagaccaatgaaatgaatgccggcaagccgctcatctccaccgaggagatgatggccctcattgtgggctacctcgcctttgttgtttttggctcgggattcatgaaggtctttgtggagaagccttttgagctcaagtacctcaagctggcccacaacattttccttactgggctttccatgtacatggccaccgagtgcgcgcgccaggcgtaccttggcggctacaagctctttggcaaccccatggagaagggcaacgaggcgcacgcaccgggtatggccaacatcatctacattttctacgtgagcaagtttctcgagttcctcgacactgtgttcatgatcctcggcaagaagtggaagcagctcagctttttgcacgtgtaccatcacgcgagcattagcttcatttggggcatcatcgctcgtttcgctcctggaggagacgcatacttttccacgattttaaacagctgcgtccatgtcatgctctacggctactacgcgtctaccacgctcggctacggctttatgcgccctcttcgtccgtacattaccacgattcagctcacgcagtttatggccatggtcgtccagtccgtctatgacttttacaacccttgcgactacccgcagcctcttgtcaagcttctcttttggtacatgctcaccatgctcggcctcttcggcaacttctttgtgcagcagtacctcaagcccaagccggccaccaagaagcaaaagaccatctaa

SEQ ID NO: 35 - DNA, 1374 bp, Botryochytrium radiatum
  misc_feature: SG1EUKS305477_( delta 6/ delta8_desaturase)
atgggtcgtggaggacaaaagacagagggactgagcgcacagccacagcaagagcgtgaacaactgctgaaaggcaagtgggaaagtgtggttcgcatcgatggtgtggagtacgatgtgacagattacatgcgcaaacacccaggtggcagcgtgatcaagtatggtctcgcaaacacaggcgcagatgccacgcacttgttcaatgcttttcacatgcgttccaagaaagccaagatggtcctcaaatcacttccaaagagacaacctcaactggaaatccaaccaggccagcttcctgaggaacaaaccaaggaagctgagatgctgagagactttgagaagcttgaaaatgaactccgtgctgaaggctattttgaaccgtcattctggcaccggttgtaccgctttactgagttggcagtgatgtttagccttggtctgtattgcttttcccttcgaacaccactatccattgctgcgggagtgtttctacatggtctctttggggcattttgtggttgggcacaacatgagggtggtcatggatctctttaccacagcttgtggtggggaaagcgtgcacaagcaatgatgattggattcggactgggaacgtcgggagacatgtggaatatgatgcacaacaagcaccatgcagcaacacaaaaggtcaaccacgacttggacattgacacgacaccattggtggcgttctttaacactgcgtttgagaaaaacagataccgtgggttcgccaagtggtgggttcgattccaggcacttacgtttcttccaatcacaagtggttgctttgtgatgtggttttggctcttgtttttgcacccacggcgtgtcgtgcaaaagggcaacgtagaggagggattctggatgttgtctagtcacattgtccgcacctatttgtttcagttgtgtacagggtgggaaagcctcgctgcatgctatttggttggttactggggcgcaatgtgggtgtctggagtgtatttgtttgggcacttttctttgtcgcacacgcaccttgacatcgtggacgcagacgtacacaagaactgggtccgctatgccgtggaccacactgtagatattagtccccagaatcctttggtgagctggatcatgggatacctcaacttgcaggtgttgcaccacttgtggcctcagatgcctcagtatcaccagcctgcggtgtccaagcgagtggctgccttttgcaaaaagcatggtctcaactaccgtgtggtctcttattttgaagcgtggaaactcatgttctccaacttgtccaacgtgagtgaccactacatgaagaacgggtttgaacgaccagccaagaagaccaaggcacagtaa

SEQ ID NO: 36 - DNA, 1377 bp, Schizochytrium sp.
  misc_feature: SG1EUKS277755_( delta6_desaturase)
atgggccgcggagggcagaaggtcgagagcggcgtgcagcagggccccgagagcgagctgctgagcaagcccgcgggctcgtgggagcaggtggtgcagatcgacggcgtcgagtatgacgtgaccaactttatgcgcaagcaccctggtggcaaggtgctgcggtacgggctggcgaacagcggggcggacgctacgcagctgttcaaggcgtttcacatgcgcagcaagaaggcgcacctgatcctcaagagcctgcccaagcggcagccccagctcaagatccagcccgggcagctgccgaccgaggagagcaaggagggcgagatgctgcgcgactttgtaaagttcgagaaggagctggaggaagaggggttcttcgagccgtcgttcgcccaccgcgtgtaccggctgggcgagcttgccgtgctgttcgcgctggggctttacctgttcactctgcgcacgccgctggccatcgccgcgggcgtggcggtgcacggcctttttggggcccggtgcggctgggtccagcacgaggcgggccacggctcgttcatgcgcagcatctggtggggcaagcgcgtgcaggccatgtgcatcgggtttggcctgggcacgtcgggcgacatgtggaacatgatgcacaacaagcaccacgccgcgacgcaaaaggtgggccacgacctggacctggacacgacgccgctggttgccttcttcaacacggcgttcgagaagaaccccaagcgcggctttagcaagtggtggacccgtttccaggcgctgacctttgtgccgatcacgagcgggtgctttgtgatgtggttctggctgctgttcctgcacccgcggcgcgtgatccagcgcggcaaggtggacgagggcctgtggatgctgagcagccatatcgtgcgcacggcgctgttcaagacgctcgccggctttgagagctgggcggcggcctatgcggtcggctactggggcgcgatgtgggtcagcggcatgtacttgtttggccacttttcgctctcgcacacgcacctcgacatcgtcgaggaggacgtgcacaagaactgggtgcggtatgcggtggaccacacggtcgacattagccccgacagctggctggtcaactggaccatgggctacctcaacctgcagaacatccaccacctgttcccgacgatgccgcagttccgccagccggaggtgtctcgccgctttgcggtcttctgcaagaagcacgggctcaactaccgcgtcgtgtcgtactggcatgcttggtacctcatgttcaacaacctcctcaccgtcggcgcgcactacgccgacaacgggctcaaccgcgacctggtcaaggccaaggccgcctga

SEQ ID NO: 37 - DNA, 1371 bp, Schizochytrium sp.
  misc_feature: SG1EUKS456777_( delta6_desaturase)
atgggccgcggagggcaaaagtcggaggccgtggccgcctcggccaccaccccgctcaagaccgcggacggcgccaagccccgctcatgggaaaaggtcgtgctcattgatggcgtcgagtacgacgtgaccaacttcatcaagcgccaccccggcggcagcgtcatcaagtacgccctcgccgaggagggcgccgatgccacggccatctacaacgccttccacatccgctcgcgcaaggccgacctcatgctcaagagcctgcccagccgcaagccccagctcgaggtccagcccggccagctcgtcgacgaggactccaaggagggcgagatgctccgcgactttgccaagtttgagcagcagctcaaggacgagggcttcttcgagccctccaacctccacgtcctctaccgcgtcaccgagctcgccgcaatctttgcgctcggcctctacctcttcagcctccgcaccccgcttgccatcctcggcggtgtcatcgcccacggcctctttggcggccgctgcggctgggtgcagcacgagggcggccacggctcgctcttcaccagcctctggctcggcaagcgtgtacaggcctgcctcatcggcttcggccttggcacctcgggcgacatgtggaacatgatgcacaacaagcaccacgccgccacccaaaaggtcaaccacgacctcgacatcgacaccacccctctcgtcgccttcttcaacaccgcctttgagaagagccgcttctcgtcggccttcaacaagttctggattcgcttccaggccttcaccttcctccctgtcaccagcggcgtgttcgtcatgctcttctggctcctcttcctccacccgcgccgcgtcatccagcgcggcctccccgaggagggcttttggatgatcaccagccacatcgttcgcactgctctcttcaaggcggcgaccggctggtcgagctgggctgcgtgctacgccgtcggctactggggttccatgtgggtttccggcatgtaccttttcggccacttttcgctttcccacacgcatctcgacgtggtggagcaggatgtccacaagaactgggtgcgctacgccgttgaccacaccgtcgacatcagccctggcaacccgctcgtttgctggatgatgggctacctcaacctccagaccatccaccacctcttcccggtcatgccgcagtacaagcaggtggaggtgtcgcgccgcttcgctgtcttctgcgacaagcacggcctcaactaccgccgctcgacctactttggcgcttggtatgatatgttcaacaacctgtggacggttggccagcactaccacgccaacggcgtccagaagaaactcaactga

SEQ ID NO: 38 - DNA, 1374 bp, Thraustochytrium sp.
  misc_feature: SG1EUKS145754_( delta 6_desaturase)
atgggtcgcggcggtcaaaacacatcaacaagcaacttggttgcttcaaaaaccatgaatggcaaggaaacccagaccgaaacgcaatgggttcgcatcaacaatgttgaatacgatattaccaactttgtaaaacgacatcctggcggaaatgtaatcaactatggactcgcaaacaccggagcagatgcgactcagttgtttaacgcgtttcacatgagatcggaaaaagcacaaaagatgctcaaaagtcttcctcaaagagaaccacaatgtgagcttcagccaggacaacttcctgaaggggatgacaaggaagcagaaatgcttcgagcattcgaaaagtttgaaaaacaacttgaagctgaaggtttcttcaaaccttctctagcacatgatatttatcgaattgtagaactcgcaggactatttcttcttgggttatactttttctctttcaaaactcctcttatgattgctgcaggagttgtcacacatggattatttggtggacgatgtggatgggttcagcacgaagctggacatggaagtttcacggaaaatttgtttctaggaaagcgaattcaagcatttttcatcggttttggacttggagcttccggatcactatggaacaaaatgcacaacaaacatcatgcagcaactcaaaaagttggacatgacatggatcttgacaccactccaatggtagcgtttttcaaagatgcatttgaaaagaatcgattcagaggattctcaaaagcatggattcaattccaagcatttactttcctccctgtcgtatctggttgttttatcatgctattctggttaacgtttcttcacccaatgcatgtcatcaaaggtggctttgttgatcaagggttttggatgctttcttcacacattattcgtgccgctttgttcaaactatgtaccggatggcaaagttgggctgcgtgttatgcagttgggtactggggatccatgtgggtctcagggatgtacttgttcggtcacttttcgctaagtcacactcatcttgatgtgattgatgctgatcagcacaaaaattgggttcgatatgctgtcgaccatactgtcaatattagtccaggaaatccgttcgtagattggatcatgggatacttgaactgtcaaatcgagcatcatttgtggcctgcaatgcctcaattccgccaaccacaagtgtccaaaagactagaagccttctgtattaaatatggtctagaatatcgtaagatgtcatacccacaagcttggtatgcaatgttctccaatttacacaatgtaggtcatcattatcatgagcatggtctgaatgaagagctcagaagcaaaaagaagcaataa

SEQ ID NO: 39 - DNA, 1218 bp, Thraustochytrium sp.
  misc_feature: SG1EUKS146174_( delta 12_desaturase)
atgtgcaagaacgaggctcagagcaagtcggcggcgcttagaggcgcgccgccgcagcagcaggaccagccgctgcccagcatcaaggacatccgcgccgccgtgcccgcgcactgctttcagcgctcggcgctgcgcagcagcctctttgtcgtgcgcgacgccgcgctcgccgccctcgtcggctgggcggcgtacaagacgctgcccaccgacctgggcaacccgctcgcgctcgccgggtggctagcctacgcgctggtgcagggcaccgtgctgaccgggctgtgggtgatcgggcacgagtgcgggcaccaggcgttctcggagagcgcgctggtcaacgactcgttcggcttcgtgatccactcggccctgctggtgccctactttagctgggcgcgcacgcacgccgtgcaccacgcgcggtgcaaccacctgctcgacggcgagacgcacgtgcccgacctcaagcgcaaggtgcacggcatgtacgccaagatcctggacgtcgtgggcgaggacgcctttgtgctcttgcagatcgtgcttcacctcctgtttggctggatcatgtacctcgtgatgcacgccacgggctcgcgccgcagccccgtgaccaaggagcgctacaagcgcaagcccaaccactttgtgccgctcgcgagcaacgagctgtttcccgccaagctgcgcttcaaggtgctcctgtcgacggtgggcgtgctgggcatgatcggcgccctttgctacgccggctcgctgtacggtggcaagatcgtctcgctgctctacgtgggcccttacctggtggtgaacgcctggctggtcacctacacgtggctgcagcacaccgacccggaggtgccgcactacggcgaggctgagtggacctggctcaagggcgctctcagcaccatcgaccgcccgtacccgtggatcgtggacgagctgcaccaccacatcggcacgacccacgtgacccaccacgtgttccacgagctgccgcactaccatgcgcaggaggccacggccgcgctcaaggctgtgctgggcccgcactaccgctacgacccgacgccgatcgtcaaggccatgtggaagaccgccgagacctgccactacgtggaggacgtgaagggcgtgcagtacctgcggtccatcgtgacggagcggcgacaggccgcgcaggcggcggccaaggccaaggcgctttaa

SEQ ID NO: 40 - DNA, 1161 bp, Ulkenia sp.
  misc_feature: SG1EUKS302483_( delta 9_desaturase)
atgacacacaattcctcttttgcaggggacgcactccatgccgatgcgtcggtgaacagagttgcaggacttgctgggaccgtggtgttcggcacagttctcgcatatcttggcactcgacagccaaaagaaagccattctatgaaacttcgtgacatgccagaagggtacaaagctcagaacgatctcgaacagaaagctatcgatgcgtacgaaagaaggcaagagctttccaccatcccatggctgtttcaaaacattcgctgggtaatgagtatctacatcggtgggatccacttgcttgccatctacgcactgccacacttgcttgactgcaagtgggaaactcttcttgggatgcttgcgttctatgtcctgggcggatttggcatcacaggtggagcccatcgactgtggagccaccgcacgtacaaagccaatgccgtatttcgattcgttgtgatgatttgcaactcgattgcaaaccaaggtaccatttaccactggagtcgtgatcatcgaacacaccacaagtacagcgagactaaggctgatccacacaacgcgcttcgaggtttcttctttgctcacgtgggctggcttttcttcaagaaagatcctcgcgtcaagtttgctggaaaccacatcccaatgaatgacctagctgcgctgccagaagtgcaactccaaaagcgcttggatccttggtggaacttgttttggtgctttggtgctccaacactttgtggtcactttctctggggagagaccgtgctgaagagttttctcctcatgggtgtgcttcgctacgccctatgcttgaacggaacctggctagtgaatagtgcagcgcacctgtatggaggccacccatacgaagacatcaatccggcagaaaacccggtcgtcgcattcttttccatgggtgaaggatggcacaattggcatcatgctttccctcacgactattctgcttcagagctaggcgtgtcgagccagttcaatccaactcggctggtcattgacttttgtgctctgtttggaattgtgtgggaccgaaaaactgcgacagaccattgggaaactagaaaaaagaagaagggctacaaacacgtggacctgcaaggtgcccctttctttcgtcagagagttgtcagctag

SEQ ID NO: 41 - DNA, 1461 bp, Arxula adeninivorans
  misc_feature: SG2EUKS136506_( delta 9_desaturase)
atgaacggtcctgaggaggttaaccttgaggaggttcaggctattgcttctggtgctgaagttcgtgctaaggtgaacattaaccgtcgtcgtcaagaagagcaagctgctgctgctgctgcatcatctggttctacaaagacccacatttctgagcaggcttttaccctcgctaactggcataagcacttcaactggattaacaccaccatcatcgccatcatccctgctatcggcttcctctctgtgcctttcattcctgtgcacggcaaaacacttgcatgggctttcgtgtactacttccttaccggcctcggcattactgctggttatcaccgtctttgggctcatcgtgcatattctgcttcttggcctcttcgcgtttttcttgctcttcttggtgcaggtgctggtgaaggttctgttaaatggtggtctaacggtcatcgtactcatcaccgctacaccgatacagacaaggacccttacaacgctaaacgtggcttctggttctctcacatgggttggatgatgttcaagcaaaaccccaagcttaagggtcgctgcgacatctcagatcttatttgcgaccccattatccgctggcaacaccgccactatatttggatcatggcagctatgagcttcgttttcccttccgtggttgctggtcttggttggggcgattatcttggcggcttcgtttttgcaggcattcttcgccagtttgttgttcatcagtctaccttttgcgtcaactctcttgctcattggcttggcgaacagccttttgacgacaaccgctctcctcgcgatcatgttcttaccgcttttgctacacttggagagggttaccacaacttccatcacgagttcccttctgattaccgcaacgccatcaagtggtaccagtacgaccctacaaagatcttcatctggaccatgaagcagcttggtcttgcatctaacctccagaccttcagccaaaacgctatcgagcagggcctcgttcaacagaagcagaagaagcttgatcgttggcgtgcacgtcttaattggggtgtccctattgaacaacttcctgtgatcgagtacgacgacttcaaggacgagtcttctagccgttctcttgttctcatttccggcattgtccacgacgttaccgacttcatcgacaagcatcctggtggtaaggccctcattaagagcgcaattggcaaggatggtacagctgtcttcaacggcggtgtttacaagcactctaatgctgctcacaaccttctcgcaacaatgcgtgttgctgtgattcgtggtggaatggaggttgaagtttggaagcgtgctcaaggcgaaaagaaggacgttgaccctgttgcagattctgcaggtgaccgcattcttcgtgcaggagatcaaccatctcgtgttcctgaagctcgtgtttctggtcgtgctgcttaa

SEQ ID NO: 42 - DNA, 1005 bp, Arxula adeninivorans
  misc_feature: SG2EUKS136495_(C16_elongase)
atgcttgaggtgacctttcctcctacccttgatcgcccttttggtgtgtacctttacggcctttttgacgctcttacaaacggttgggctactcgctttcagtttgcacaggatagcggcattcctttctcttctcgttgggaggttgcagctggcattgtcacatactacgtcgttatttttggtggccgcgaggttcttaagaacgctcctgttattcgcctcaacttcgtgttccagatccacaacctcatccttaccctcctttctcttggccttctccttctcctcgttgaacagctcatccctatcattgtccgtcatggtgtgctctacgcaatctgcaacagcggctcttggactcagcctattgttacagtgtactacctcaactacctcaccaagtactacgagctctttgataccgtcttcctcgtgcttcgcaagaaacctctcaccttccttcacacctaccatcacggcgctacagctcttctctgctttacacagcttattggccacacctctgtctcttgggttcctatcgtgctcaacctcttcgttcacgtcatcatgtactactactacttcctctctgcacttggcgttcgcaatatttggtggaaagagtgggtcacacgcacacagatcatccagttcgttgttgacctcgtcttcgtctacttcgctacctacacctacttcaccaacaagtactggccttggctccctaacaagggcacctgtgcaggcgaagagtttgccgctatttatggttgtgcacttcttacctcttacctcttcctcttcatcgctttctacattcgcgtctacactaaggcaaaggcaaagggccgtaaacgtgctgcttctgctgctgctaaggcaacaactggtgtcgttactgctgatcgtccttctacccctattgctaccactaacggcgctgctacaggtgctgcaggtgctacaggttctgttaaatctcgttctcgtaaggcttaa

SEQ ID NO: 43 - DNA, 1137 bp, Oblongichytrium sp.
  misc_feature: SG1EUKS299580_(C16_elongase)
atggcaacttccatgaacgattcactcccggagggggccacggccatgaacacattgcagcgcctcttgtccttccaaaacgagtttcatagcaaggaggtacttacatggcatcgtgaccatgcagagatcccaatcatttgcctttccctgtatctggtcatggtgttcgcgggccccgacttgatgaaacatcgcgaacctttcaagctcaagcgcacctttgcagcgtggaactttttcctctcagtctttagcatccttggggcatatcacatggtaccacttctattggggaggctttgggaacatgggttcaaagccacagtatgcagccaccccgaatggtactttaatggaccaagcggcttttggctctgtctattcatttacagcaagtttgcggaattgttcgacacggcgttcctcgtcctccgcaagcgaaatgtaatttttctgcactggtttcatcatgcaaccgtactgctgtactgctggcatgcataccaccatgcaattggtgcaggtgtttggttcgcgtgcatgaactactgtgtccattctgtcatgtatttttattactttatgaccaatgttgggctctatcgcgtagttgcaccctttgcccaagcaatcaccactgtgcaaattctacaaatggtcggtggtatggctgtattgttgcaggtggcgcatgcgcgtttgaccgaagaccctacggcatgtgaggtggattccgcaaattggaaacttggacttgcaatgtatgcgtcctatttcgtgctctttgtactgctctttgtgaagaaatacctgtctccagcgccatcgtcagcccaatccaagcgcacaagtggaaacgacacctcttcggccactggttcgaccaccaagagcaattcatcgtcgacaaagggggatgcagatgcttcggcaggtgctcgtagacgagctcgggtctcggtaacttgcccccctggcctggacgatgtccctgtctttggtgacagtgcaagtgtaagcaagttggatgcgtctggctttttccattcacagcagactgtggaagaagcacgaagagcccaggatgatgctgaccgagcgcaggcaatgcgaaacaagaagaaggcgctttag

SEQ ID NO: 44 - DNA, 942 bp, Oblongichytrium sp.
  misc_feature: SG1EUKS133590_( delta5_elongase)
atgtcgatttctgaacaagcagctgcgggcgcgcccacgaagccgcttccatgcaaggatgctgtcgattctgcaaatgctgcagctcgcatcctcaaaaaggaggaatccgtctcgagctttcagtggcaaaaggccctctgtattgttgccatggagctctacttgatgaagatgatctttgatgagtcaaaaaacaaggtcaacccctttggagaggcaagctgggtgtacccacttgttggtaccgtttgctacctgctctttgttttcttgggcaagaaatacatggcaactcgtgaagctttcaacgtcaagcagtacatgatcatgtacaacttgtaccagacggtgtttaacgtatacgttgtagtttgcttctttcaagaaatttaccgtcgtaagcttccagctgccggaacgaaattcgttcctggtccagaagaattcaaccttggcttccttatctatgctcattaccaaaacaaatatcttgaactcctggacaccgtctttatggttgtgcgcaaaaagaacaaccagatctcgttcttgcatgtctaccaccacacacttttgatctggtcctggtatgcagtgctcaagattaacccaggcggtgatgcttactttggtgctcttgctaactcgatcatccacgttgtcatgtacagctactatttgctcgcacttctcggtgtcccatgcccatggaagaagtatgtgaccattatgcaattggttcagttcatggttgtgttcagccaaggtttgtactgcttgtacttgggctcaacaccaaccatgctcattcttcttcaacagtttgtcatgatcaacatgcttgtcctgttcggcaacttctacatgaagtcctacaggaaaaaggccgctgaccgcaaggccaaggctgaagaagagaacacaaaaaagaccgagtag

SEQ ID NO: 45 - DNA, 939 bp, Oblongichytrium sp.
  misc_feature: SG1EUKS329508_( delta5_elongase)
atggcagctcgagtggacgcaaagggtgtacaagccaaaacgcaagtggcgaagactgtgccacgttccaggaaagtagaccgcagcgacgggttttttaggacgttcaacctctgcgctttgtactgctccgcttttgcatacgcgtacaagaacggtccaacagacaatgacgaaaacggattgttcttttccaaatctcctttctatgccttcttggtatcggacgctatgacctttggcgccccactggcatatgttgtagcagtgatgctccttagtcgttatatggctgacaagaaaccgatgactggattcatcaagacgtatgtgcagccagtgtacaatgtagtgcagatcgttgtgtgtgggtggatggcttggggcctccttccacaagtaacactgaccaaccctttcggactcaacacacagcgtgaccctcagattgaattctttgtgatggtccatcttcttaccaagtttttggactggtctgacacctttatgatgatcctgaagaagaactatgctcaggtttcattcttgcaagtgtttcaccacgccacgatcggcatggtttggtctttcttgctccagcgtggatggggctctggaactgcagcatacggggcattcatcaactctgtgactcatgttattatgtacacgcactactttgtcacatctctcaacatcaacaatccattcaagaggtacatcacagccttccagctgtctcagtttgcttcctgcatcgtgcacgcagtgcttgcgctgttgtttgaggaagtatacccaatcgagtacgcgtacttgcagatcagctaccacatgatcatgctctacttgtttggctgccgcatggactggagtcctctgtggtgcactggcgaagtagacgggttggacgaggcgaacaagaagaagacgaactaa

SEQ ID NO: 46 - DNA, 1533 bp, Thraustochytrium sp.
  misc_feature: SG1EUKS132052_( delta4_desaturase)
atgggagaagagaagaagatcacccttgcgcaagtacgtgagcacaatctgccaacggatgcctggtgcgtgatccacgacaaagtgtacgatgtaacaaactttgccaaaatacacccaggtggtgaccttgtgttgcttgctgccggaaaagacgcaactattttatacgaaacgtaccacattcgtggtgtacctgatgccgtgcttgcaaagtacaggataggaactttggaagcaccagaggttaagtcgaccagtggtctggacagtgcatcctactacagttgggacagcgaattctacaaagtgctcaagaagcgcgtcgtggctcgtttagaaaagctaaagttggagcgcagaggaggtatcgagatctggaccaaagctttcatgctcatgactggtttctggggctctctctacttgatgtgcacgcttaaccctgacggttgggccattccagctgctatgagcgttggagtttttgctgcatttgttggaacttgcattcagcatgacggaaaccacggtgctttcgcaaagtcaaaattcttgaacaaggctgcgggttggacgttagatatgattggcgcaagtgcaatgacttgggagatgcagcacgtgttaggccatcacccgtacaccaacttgattgagatggagaacggagtccaaaaggtcagcggcaagccagtggacaccaagaaggtcgaccaggaaagcgacccggatgtgtttagcacctacccgatgcttcgccttcacccatggcataggaaacgcttttaccacaagtttcagcacatctatgcaccattcatcttcggcttcatgacgatcaacaaagtcattacccaggatctcggcgtgcttctcaacaagcgcttgttccagatcgatgccaactgtcgatatgccagcccagggtatgtctttcgtttctggttcatgaagtttcttaccatgctttacatggtaggactccctatgtacatgcaaggaccactccaaggcctcaagctttactttgtagcacactttacctgcggagagcttcttgcaaccatgtttatcgtgaaccatatcatcgaaggcgtgagttacgccagcaaagacgctgtgaaaggaggcatggcccctccacgaactgtccatggagttacgccaatgaatgagactcaacaggctctggagaagaaagagaagcttgcacctacaaagaagatccccctcaacgactgggctgcggtacagtgccagacttctgtcaactgggcaatcggttcgtggttttggaatcacttttctggcggtctcaaccaccagatcgagcaccatctcttccctggtctcacgcacaccacctacgtgtacatccacgatgtcgttaaggatacttgtgcagagtacggtgttccgtaccagcacgaggagagcttgtactctgcctactggaaaatgttgtcgcatctcaagacgcttggtaatgagccaatgcctgcttgggagaaggaccatccaaaggcttga

SEQ ID NO: 47 - DNA, 1311 bp, Oblongichytrium sp.
  misc_feature: SG1EUKS529637_(omega3 desaturase)
atgtgtaacccaaagagcttgagtgcacccagtggcgttgaggcgcatgttgtacaagagcatgtgcccgttggcgagcatgacaagtggttggaaacacttgactttgatgcgttcaaggaggacatgcagcggttggggaaatccctcgcggacggtcaggggcaacaagacgtggaccacctccaaaagtttgtgtggtggaaccgcatcgtgacgctctgtggaatccttaccatggcttgcttccccaacccctttaccgtgttttgcttgagccttgggaccttttcccggtggaccatcattgcgcaccacgtctgccacggcgggtttgacaaggccgacaagtccaagcggtacaaccggtttacgtttggggtgggctccctctaccgccgcatggtggactggttcgactggatgctccccgaggcgtggaacgtggagcacaaccagatgcaccactaccacctcaacgaggccaaggacccggacctcgtggagctgaacctttccccgctgcgggagaacgacgcggtgcccatgcccgtcaagtacatggtggtgttcttctttatgtgcacctggaagtggttttactacgcgcccagcacgtttattcagctcgcccaggcccggcttcgccgccagggcgtctcgacggagggcatgccgcgcgcccacctgacgatcgcctcaattttcgaggcccccgactttgtgagcatcaaggagctgttcttgcgcgtgctgttcccgtactttgcgtaccgcttcctcctggtcccgctccccgtgctcgcactttggggcccaaaactgttttcctatgccatcatcaacctcgtcctggcggacattgtgaccaacatccactcgttcattgcggtggtcaccaaccacgccggggacgacctctacgtgtttgaaaagcactgcaagcctcgtggggcacatttttacctccgccaggtcatcagctccgtggactttacgtgcggcaacgacctcatggactttatgcacggcttcctcaactaccagatcgagcaccacctttggcccgatctttccatgctttcttatcagcgtgcccagcccctcgtcaagcagctctgtgccaagcacggcgtgccttatgtccaggagtcggtgtggatccgtctgaaaaagacgatcgacattatggtgggcaagacctcgcaccgcaagtttccctcccaatacgagccaaacgactacgaccacctcaaaaccgccgccgccaccaccacggccatggaggaacccgaagccgaggcgaaataa

SEQ ID NO: 48 - DNA, 1299 bp, Oblongichytrium sp.
  misc_feature: SG1EUKS109359_(omega3 desaturase)
atgtgccgagcacaggtcgctgctgaagcgacggttggcgagccaatggccgaaccatcgtcaaatgagcctgccaatgttagcaagttggcgccagaagaccgctgggttgaaacccttgatctggatggcttcaaggaagaaatccaggcgctcggaaaggagctcgccgcgaaccaaggcgaagatgatgttcgccacctgaacaagattctttggtggaacaccatcctcaccgtgctcgggatcggaacgatgtgggcctccccaaatctgtttaccatcatctgtcttagtgtggcgacattttctcgctggacgatgattggtcaccacacttgccacggtggttatgataagtgtgaaccatcgggacgattcaaccgctttaagtttgctgttggtagcctttatcgccgcgctgtagattggtttgactggatgttgcccgaggcgtggaacatcgagcacaaccagctccaccattaccatcttaacgaagccaaggaccctgaccttgtccaggaaaacctctcatatttgcgtgacttgaacatcccagtagcactcaagtatgtggtggttgcctttttcatggggacatggaagtggttctactatgcaccaaacacatttcaccaactcgaaattgcacgccttcgccgtgaaggtggcgaagtgagcaagcttgagcgagttcacgtcactgtagcctcgcttgccgaagctcctgaatattacaacgtaaccaagctcttcacccatgtgcttggcccatactttgtgtaccgctttgtactcatgcctctcccgattttggtcttgctgggccctacttatttctacaatgcgatcatcaacctggtgctcgctgatattctgacaaacattcatggctttattgccattgttaccaatcacgcaggggacgacctgtacacttttgaaaagcactgtaagccacgtggtgcgcacttttacttgcgacaagtcattagctctgtagacttcgcttgcggcaacgatctcgtcgattttcttcacggatggctcaactaccagattgagcaccatctctggccagatctctccatgttgagctaccaacgatcgcaaccacgtgtgaaggccatctgtgccaagtacggcgtgccattcgttcaggaaagcgtctggattcgcctcaagaagactatcgatattatggttggaaactcgaccatgcgaccatttccaattgagttcgaaccaaacgattatgacgacgttgcaaccaatggaacaaagaaggaaaactaa

SEQ ID NO: 49 - DNA, 1092 bp, Pavlova pinguis
  misc_feature: SG1EUKS224768_co886_(omega3 desaturase)
atgtgtcctcctgctactcatgatgctacccctatcaaggatggtgctaatcgtgctgagatcgttgctgagtctaagcttacccttcaggatatccgcaaggccattcctcaagagtgcttcgagaagaacactgctcgctctatgctctaccttgttcgcgacctcgctatttgcgctactgctcctcttgtttacccttacgttgctgcttctggtaaccctcttgcttatctcgcttactggaacttctacggcttcttcatgtggtgcctctttgttgttggacacgattgcggccacactaccttttctcccaacaagacccttaacgacatttgcggccacattgctcacgctcctcttatggtcccttactacccttgggctatgtctcatcgtcgtcatcacatgtaccacaaccaccaaaagaaggacgcttctcatccttggttcagcaagtcttccctcaagaaacttcctgcttttacccgcaacttcctcaagagccctcttgccccttttctcgcttaccctatctacctcttcgaaggcagctttgacggttctcacgtgttccctctctctaagctctacaagggctctcaaatgcgtgctcgtgttgaatgcgctatttctgcagttaccgtgttcgcttttggcactgctgcttacatgttttgtggcgatgctcgtactcttgcacttgcttacggtggttgctatgcttgcttctctttctggctcttcatggtcacctacctccagcaccacgatcatggcactctcgtttacgacgactctgattggacctaccttaaaggcgctcttgagactgttgatcgcaaatacggctttggcctcgacaaccttcatcacaacattagcgacggtcacgttgttcaccacctcttctttacccaagttcctcattaccacctcacaaaggctaccgagcaggttgctcctcttctccgtaaggctggtgtgtacaagcgtgttgaccacgacaatttccttaaggacttttggcgcacctttttcacatgcaacttcaccggctggaaatgggctaatggcaaggacaactga

SEQ ID NO: 50 - DNA, 1194 bp, Oblongichytrium sp.
  misc_feature: SG1EUKS102142_(delta12_desaturase)
atggctaatttgtcccccatggatcaagagcgtttgtcggagattactccgttcaagcctacggtggaccacccagtgccaaccatcaagcaagttcgcgatgcaattcctgcagaatgctttgaacgcaaactatggaagggtcttgcgcttgtcgtgcgcgatggtatcattattgctggtcttggctttgcggcatggcagttgccgttcgcgaacttgactcccgcaatgatggcagtctggcttgtctatgccgttcttcaaggaactgcactcacaggatggtgggtccttgcacacgagtgcggacacggtgcatttagtagctactcgtggatcaacgataccatcggctttgtactccacaccgtactgttagtgccgtatttctcatggcaatactctcatggcaagcatcacgctaagacgaatcaccttctcgatggtgagacacatgtgccaccaaagggtgtacgaggccttaacgcaaagcttgctgaccttctgggcgaggatgcattcgcgatgtggcagctagtttcgcatttggttttcggttggccactttacctcatcatgaatgcaactggagcacgacgtcgctttgatcgtgtacggtactctagcagcccaagtcacttttcaccaacctctgaactgtttccatcacgatggcgcccttgggtagccttatccagtgtcgggatcattgcttggctcactgtattgtatgtggtcagcacacatattggtacacagcgcttggctctcatgtacgggttcccttaccttgttgtcaacggttggctcgtattgtacacgtggcttcagcatgttaatgaatatgtaccgcagtatggggaagacgaatggacatggatgaagggagcacttgcaactgtcgatcgtccgtactgggggcccgtcgactggatgcatcaccacattggcacaacacatgtagcccaccacgtgttttctcacatgccttgctacaatgcacctcgtgcaaccatcttcttgaaacgcttccttggtgatctttaccactatgatgaacggccgatttccaaagctgcatggcaggtcgcaaaagaatgtcactacgttgatgatttgcaaggtcgtcagctcatgaaatcaatcttcaagcaccacaagaaagaggcgagcaagaaacgcaatcaatag

SEQ ID NO: 51 - DNA, 1317 bp, Nannochloropsis oceanica
  misc_feature: EMRE1EUKT5767207_co886_(delta12_desaturase)
atgggtagaggtggtgagaagactgttacccctcttcgcaagaagacccttcttgatgctgcaagcaccatttctggcactgttcgtccttctaaggctgttgaggctcttcctactgaggaacttcgtaagaaggctgctcaatacggcattaacaccagcgttgatcgcgagactcttcttcgtgaacttgctccttacggcgatatccttcttcgcaacgatgctcctaagtctctccctcttgctcctcctccttttaccctttccgatatcaagaacgcagttccacgtcactgctttgagcgctctctttctacctctctctttcacctcaccattgacctcatccaggttgctgttcttggctaccttgcttctcttcttggccactccgatgttcctcctatgtctcgctacattctttggcctctctactggtacgcacagggttctgtccttacaggtgtttgggttattgcacacgaatgcggacaccagtctttctctccttacgagtctgttaacaacttcttcggctggcttctccactctgctcttctcgttccttaccattcttggcgcatttctcacggcaagcaccacaacaacactggttcttgcgagaacgatgaggtgttcgctcctcctatcaaggaagagctcatggatgagattcttcttcactcccctctcgcaaaccttgtccagatcatcatcatgctcactattggctggatgcctggctaccttctccttaacgcaactggtcctcgcaagtataagggtctcagcaactctcacttcaacccaaacagcgctctttttagccctaaggaccgcctcgacattatctggtccgatatcggcttttttgttgctctcgcttgcgtcgtttacgcatgcgttcaatttggctttcaaaccgtgggcaagtactacctcctcccttacatggtcgtcaactaccacctcgttctcatcacctaccttcagcacacagacgtcttcatccctcattttcgtggttctgagtggacctggtttcgtggcgctctttgcacagttgatcgttcttttggctggcttcttgatcacacctttcaccacatttctgatacccacgtttgccaccacatcttcagcaagatgcccttctaccacgctcaggaggcttcagagcatattcgcaaggcacttggtgactactaccttaaggacgatacccctatctggaaggctctttggcgctcttacaccctttgcaagtacgttgactctgaggagactaccgttttttacaagcaacgcgcttag

SEQ ID NO: 52 - DNA, 1161 bp, Isochrysis sp.
  misc_feature: SG1EUKT1012028_co886_(delta12_desaturase)
atgggtaagggtggttctgctggtaagtcttctgctgttgagcgtcttggtcctcttgctgctacaattcctgagccttctaaggagatcacaaagggctctattcgtgctgctattcctcctcacctctttcagcgctcttacgttcactctcttggtcaccttgtgatggatcttctctgggttgctgcaacatggcttcttgcacttcaggctgcttctgttcttccttctggctttgctcctcttgtgtgggctgcatactggttctaccaaggcattaaccttaccgctctttgggttcttgctcatgaatgcggtcatggcggcttcactgattctcgtcttgttaacgacattgtgggttttgtgcttcactccgctctccttaccccttacttctcttgggctatcactcacgctaagcaccaccactacaccaaccacatgaccaatggcgagacatgggtcccttctacagctaatcctgaaaaggcttctgttaagtttgcaaagtcccccatcggcaccattcgccgcattgttgttgttgctcttcttggatggtacacctacctcttcaccaacgctactggagctaagcaaaatgctggccaaagccacttcagcccttcttcacgtgctcttttcaaggctaaggacgccaaccttgttcgtgcatctaacctcggaatgattgcttgtcttgcagttctcgccaagtgcgtttccgtttggggtttttctgctgtgttcttcaactacctcgtccctcagaccatctgcaacttctacctttgcgctatcaccttcatgcaacacacccacgagtctgtccctcactttaactctgaggagtggacctggcttcgtggtgctctttctaccattgatcgttctatgggttctcacgtcgattggcgtcttcaccacattgttgactctcacgttgttcaccacatcttctccgacatgcctttttacggcgctaaagctgctaccccttacgttaaggagcacctcggcatctactacaagagcaaccttggctctaaggtcctcggttctgagtatctcggctattggaaggatttctactctagcatgtctcgcgctgtcgttgttggtgttggtgaggacaactttatgtggtttcgctga

SEQ ID NO: 53 - DNA, 997 bp, Aurantiochytrium sp.
  misc_feature: Tub-alpha-997 promoter
ggagggaggcatgaaaacaaagttgttaaaactaaacgaggcgaaagaaagcggcggagcaatggtagacacaatatgagtaggtagcaggtccctacagccagccactcaatcatacgaggagtctatgcaggggtgctagtattttatagtatctggatgctaagaggcacgacttcttccgcctgtctgtttgtctatctggctacccctattgtatcttatatcctaaacactagggctccctcaccagcaacagtcagtcactcacgcattcagtttatactaaggctcacctagactaatccataagcagccaatccgttccgcgctcgcgccggtagaagcaaccggaccatacggaggtcttaatgtttaggttatatggactatgtcttatcggtgggccgttatacacgccgcgctggaagctcctctactttgtgaggagtttcacttataatgaatgatcgggattcctgttcccctcccatccactgggtgcaaaactcaactccctcacaaaaagtgtattctataaatatatgtaaaagcaacggtcgctacctctaagtacactgatgatataaacaagagcaagatggaagttttcagtgtttgttgtgaggaacagcactggaggccaaaacaagcctcttagaaagttctccactggcaagcttcgacggtttggcgcagagtgagggcagcaaactttgccgcatcgcagcaaatctcaatcagccttttgacggtcgtgcctaacaacacgccgttcaccccaagccttactttgccttcgtgcattgtcctcgagtatcgtaagtttgattcgctttcattcgcttccatccactccggttgtagcaaaagcaaagcagcgttgtgcggctctcaaggtttggccctgatgcgaccgacgagcataaactaactagcctccgtcttggtttcgtttcacagtaaagtagttttcgaaactccaacctcaagcaaa

SEQ ID NO: 54 - DNA, 247 bp, Saccharomyces cerevisiae
  misc_feature: PGK1 terminator
attgaattgaattgaaatcgatagatcaatttttttcttttctctttccccatcctttacgctaaaataatagtttattttattttttgaatattttttatttatatacgtatatatagactattatttatcttttaatgattattaagatttttattaaaaaaaaattcgctcctcttttaatgcctttatgcagtttttttttcccattcgatatttctatgttcgggttcagcgtattttaagt